(19) United States
(12) Patent Application Publication
(10) Pub. No.: US 2013/0226285 A1
(43) Pub. Date: Aug. 29, 2013

(54) PERCUTANEOUS HEART BYPASS GRAFT SURGERY APPARATUS AND METHOD

(71) Applicant: Gera Strommer, Haifa (IL)

(72) Inventor: Gera Strommer, Haifa (IL)

(21) Appl. No.: 13/884,727

(22) PCT Filed: Nov. 10, 2011
(86) PCT No.: PCT/IL11/00873

§ 371 Date: May 10, 2013

Related U.S. Application Data
(60) Provisional application No. 61/412,991, filed on Nov. 12, 2010.
(60) Provisional application No. 61/558,150, filed on Nov. 10, 2011.

(51) Int. Cl. A61F 2/958

(52) U.S. Cl. CPC A61F 2/958
USPC 623/1.23

(57) ABSTRACT

Apparatus for performing bypass graft surgery on a patient, between a major blood vessel and an occluded vessel in the patient, the vessel being occluded by a lesion, including a blocking balloon, an endoscopic delivery device, a distal suture structure, a proximal suture structure and a delivery catheter, the blocking balloon for isolating a portion of the major blood vessel, the endoscopic delivery device for grasping a vessel graft and for navigating the vessel graft to the major blood vessel and the occluded vessel, the distal suture structure positioned within the distal end of the vessel graft, for suturing the vessel graft with the occluded vessel at a position distal to the lesion, the proximal suture structure positioned within the proximal end of the vessel graft, for suturing the vessel graft with the major blood vessel and the delivery catheter positioned within the vessel graft, for holding and releasing the distal suture structure and the proximal suturing structure.

### FIELD OF THE DISCLOSED TECHNIQUE

The disclosed technique relates to heart bypass graft surgery, in general, and to methods and systems for performing off-pump, catheter-based, percutaneous heart bypass graft surgery, in particular.

### BACKGROUND OF THE DISCLOSED TECHNIQUE

The heart supplies blood to organs and tissues of the body through blood vessels. Arteries are blood vessels transporting blood from the heart to various organs and tissues of the body whereas veins return blood from the body back to the heart. Besides supplying blood to the body and to all the organs in the body, the heart supplies blood to the muscles and tissues of the heart through blood vessels known as coronary arteries. Blood supplied to the heart tissue is returned to the right atrium of the heart via a group of veins known collectively as the coronary veins and are drained by the coronary sinus. Heart disease represents a broad term for classifying various types of diseases that affect the heart. One type of heart disease, known as coronary heart disease, refers to the failure of the heart and its surrounding tissues to receive adequate blood circulation. Coronary heart disease is most commonly associated with atherosclerosis, in which the inner wall of one or more of the coronary arteries thickens due to a build-up of plaque materials, such as lipids and fatty acids or lesions.

Reference is now made to FIG. 1A, which is a schematic illustration of a human heart with a blocked coronary artery, as known in the prior art, generally referenced 10. Heart 10 includes an aorta 12, a pulmonary artery 14, a superior vena cava 16, an descending aorta 18, a right coronary artery 20, a left main coronary artery 22, a left anterior descending (herein abbreviated LAD) coronary artery 24 and a network of smaller coronary arteries 25, a brachiocephalic artery 26, a left common carotid artery 28 and a left subclavian artery 30. Heart 10 also includes a lesion 32 blocking LAD coronary artery 24 and limiting blood flow within LAD coronary artery 24 and network of smaller coronary arteries 25. Aorta 12 is the main artery exiting heart 10 which delivers blood to the body (not shown). Pulmonary artery 14 delivers blood to the lungs (not shown). Aorta 12 branches out into brachiocephalic artery 26, left common carotid artery 28, left subclavian artery 30 and descending aorta 18. Brachiocephalic artery 26, left common carotid artery 28 and left subclavian artery 30 supply blood to the upper body, including the arms and the head. Descending aorta 18 descends behind pulmonary artery 14 and supplies blood to the lower body. Right coronary artery 20 and left main coronary artery 22 branch out into LAD coronary artery 24 and a circumflex artery (not shown), which are the main arteries supplying blood to heart 10. These arteries subsequently branch off into network of smaller coronary arteries 25. Lesion 32 is located in LAD coronary artery 24, although it is noted that lesion 32 can be located in any one of the coronary arteries of heart 10. Lesion 32 substantially reduces the flow of blood, or occludes the flow of blood completely, to LAD coronary artery 24 and to network of smaller coronary arteries 25 which branch off of left main coronary artery 22. In general, if lesion 32 blocks at least one of the main coronary arteries, such as right coronary artery 20, left main coronary artery 22, LAD coronary artery 24 or the left circumflex artery (not labeled), the reduced or occluded flow of blood to heart 10 may cause various types of coronary heart disease, such as an angina or a heart attack.

Surgical procedures and devices are known for treating coronary heart disease. One known set of procedures is percutaneous coronary intervention (herein abbreviated PCI), also known as coronary angioplasty. PCI involves inserting various devices, such as wires, balloons, catheters, stents and the like, through a small incision in the skin into a major blood vessel, such as the femoral artery. The device or devices are then advanced through the aorta and into the relevant coronary artery, to the location of the lesion blocking one or more of the coronary blood vessels of the heart. The devices are then used in an attempt to remove the lesion, open the lesion, widen the walls of an occluded coronary blood vessel, or vessels, and the like. Whereas PCI is a form of minimally invasive surgery that involves a lower surgical risk to a patient and a substantially shorter recovery time, in various scenarios of coronary heart disease, PCI is not a viable or practical option. For example, in cases of coronary heart disease such as left main coronary artery disease, that includes complex lesions and chronic total occlusion (herein abbreviated CTO), or in cases where a patient is diabetic or suffers from kidney malfunction, PCI is not usually considered as a form of treatment.

In such cases, known surgical procedures may be performed such as coronary artery bypass graft surgery, usually abbreviated as CABG surgery. CABG surgery is used to treat and fix occluded coronary blood vessels by providing an alternative route for blood to flow from a location proximal to the occluded coronary blood vessel to a location distal to the occluded coronary blood vessel. In general, CABG surgery involves open heart surgery and can be performed on-pump or off-pump. In on-pump CABG surgery, a patient's heart is stopped and connected to a heart-lung machine. The heart-lung machine substantially takes over the role of heart by circulating blood and oxygen to various parts of the body while the heart is drained of blood and operated on. In off-pump CABG surgery, a patient's heart is operated on while still beating and circulating blood to the body. In either case, in CABG surgery alternative routes for blood to flow around an occluded coronary blood vessel are created. These alternative routes may be blood vessel grafts, which are either artificial or harvested from the patient. One side of the blood vessel graft is sutured to the aorta while the other side is sutured to a point which is distal relative to the occluded coronary blood vessel, i.e. beyond the occlusion, thereby providing an alternative route for blood to reach the coronary vessels of the heart. An alternative route can also be created by redirecting an existing artery (usually the left internal mammary artery, usually abbreviated as LIMA) and suturing it to a point beyond the occluded coronary blood vessels. In general, open heart surgery involves increased surgical risks to a patient and a substantially long recovery time. Also, open heart surgery usually requires a patient to be sedated for hours, for example between 3-5 hours and usually requires a large surgical team.

Other approaches to performing heart surgery and CABG surgery in particular are known which use endoscopic apparatuses and involve less invasive techniques. Such approaches use a combination of a robotic system and multiple endoscopes to perform the surgery. An example of such a system includes the da Vinci Surgical System from Intuitive Surgical, Inc. Where such approaches to do not involve open heart surgery, such approaches are expensive to install and operate and usually require surgeons to undergo extensive training and specialization before using with such robotic systems in performing heart surgery. Also such approaches may require a large surgical team and may require a patient to be sedated for several hours.

Devices and methods for performing percutaneous CABG surgery are known and are described in the following patents and patent application publications. U.S. Pat. No. 6,475,226 to Belef, et al., entitled “Percutaneous bypass apparatus and method” is directed towards devices and methods for percutaneous translumenal minimally invasive coronary surgery. The methods according to Belef include the following steps: determining a proper location for treatment, navigating a suitable device to the treatment site, creating an extravascular opening and pathway, guiding and/or monitoring the progress of creating the opening and pathway and maintaining the extravascular opening and pathway. Extravascular openings and/or pathways can be created to define a fluid path or bypass around a vascular restriction in a vessel wall by using an intravascular catheter. The intravascular catheter includes an elongate shaft adapted for intravascular navigation, an anchoring mechanism disposed on the distal end of the shaft and a tissue penetrating member. The tissue penetrating member has a proximal end slidably disposed in the shaft and a distal end including a tissue penetrating mechanism. The tissue penetrating member is extendable between a retracted position and a penetrating position wherein the tissue penetrating mechanism extends completely through the vessel wall to establish an extravascular opening therethrough. The catheter may include a stiffening member slidably disposed about the tissue penetrating member for providing rigidity to the distal portion.

U.S. Pat. No. 7,004,175 to LaFontaine, et al., entitled “System and method for percutaneous coronary artery bypass” is directed towards a percutaneous system for bypassing a restriction in a native vessel of a mammal having an aorta. The system includes a graft having a body portion with a first end, a second end and a lumen therebetween. According to the system of LaFontaine, an aperture is formed in the aorta with the graft being inserted into the aorta. The first end of the graft is connected to the aorta about the aperture in the aorta. Another aperture is then formed in the native vessel distal of the restriction. The second end of the graft is connected to the native vessel about the aperture therein such that the lumen in the graft communicates with the aorta and the native vessel.

U.S. Pat. No. 6,309,416 to Swanson, et al., entitled “Medical anastomosis apparatus” is directed towards a connector for use in providing an anastomotic connection between two tubular body fluid conduits in a patient. The connector can be a single, integral, plastically deformable structure that can be cut from a tube. The connector has axial spaced portions that include members that are radially outwardly deflectable from other portions of the connector. The connector is enlargeable in an annular direction so that it can be initially delivered and installed in the patient in a relatively small annular size and then enlarged in the annular direction to provide the completed anastomosis. The connector can be enlarged by inflation of a balloon placed temporarily inside the connector. The radially outwardly deflected members of the first and second portions respectively engage the two body fluid conduits connected at the anastomosis and hold those two conduits together in fluid-tight (i.e., from which body fluid does not leak) engagement.

U.S. Patent Application Publication No. 2010/0069820 to Zotz, entitled “Device, system, kit, and method for epicardial access” is directed towards equipment for the performance of minimally-intensive body access, such as cardiac access. According to the system of Zotz, a coronary artery bypass can be created by creating a direct path of flow from one body artery into a coronary artery with the aid of catheter-based minimally-invasive epicedial access bypass surgery. In this surgery, various medical devices are used, such as a special partially flexible needle, a special partially covered stent and a special partially flexible port. In addition, the system of Zotz includes a balloon catheter having an inflatable, expandable balloon centrally arranged at a distal end thereof. A length of a blood tight tubular structure is arranged coaxially around the balloon along a defined length at the distal end. At least one expandable fixation unit is arranged coaxially between the tubular structure and the balloon and at least one restriction unit is arranged coaxially around the tubular structure.

U.S. Patent Application Publication No. 2005/0182431 to Hausen, et al., entitled “Tool and method for minimally invasive bypass surgery” is directed towards a method for performing minimally invasive coronary artery bypass graft surgery using a splittable proximal anastomosis tool as well as a distal anastomosis tool. The splittable proximal anastomosis tool is adapted to split after deploying an anastomotic device at the proximal anastomosis site. By splitting the tool, a graft vessel can be released within the thoracic cavity of a patient after performing the anastomosis at the proximal site. In addition, by splitting the tool, a distal clamp can be placed on the graft vessel before the proximal anastomosis is performed, facilitating the procedure and allowing distal anastomosis to be performed first if desired. The distal anastomosis tool includes a staple holder having two spaced-apart arms, staples detachably held by the staple holder and an anvil connected to the staple holder. The method and system of Hausen is assigned to Cardica, Inc., which manufactures systems for automated anastomosis, such as their PAS-Port® system for proximal anastomosis and their C-Port® systems for distal anastomosis, viewable at the website http://www.cardica.com/index.php?option=com_content&task=view&id=33&Itemid=84.

U.S. Patent Application Publication No. 2003/0144676 to Koster, Jr., entitled “Anastomosis device and method” is directed towards a device for anastomosis of a vein graft to an aorta. The device is used for creating a circular hole in the aorta wall, occluding the hole to prevent blood loss and providing a guide for insertion or placement of the vein into the hole in the aorta wall. The device includes an elongated main body housing a punch mechanism. The punch mechanism has a disk-shaped punch head and a tubular cutting sleeve, which in conjunction act to remove a circular plug from the aorta wall. The punch head is retractable relative to the cutting sleeve, with the cutting sleeve remaining in the aorta wall to prevent blood loss. The device also includes an obliquely connected lateral shaft having an internal bore communicating with the main bore of the cutting sleeve, whereby a vein graft can be introduced into the main bore and through the aorta wall. A portion of the cutting sleeve remains disposed in the aorta wall to prevent blood loss during the insertion of the vein graft. Anchoring means for the vein graft is also included and comprises an expandable annular wire lattice having short radial projections to secure the lattice in the interior of the vein. The anchoring means also comprises longer, flexible prongs which are compressed against the outer wall of the vein while resident within the lateral shaft and during passage through the cutting sleeve portion of the device. These longer flexible prongs automatically expand radially to prevent withdrawal of the vein from the aorta wall after the anchoring means is fully inserted and extended from the distal end of the cutting sleeve. The anchoring means is secured to the vein prior to insertion into the lateral sleeve, and is advanced through the lateral shaft and cutting sleeve bore by use of a balloon catheter.

U.S. Patent Application Publication No. 2007/0276462 to Iancea, et al., entitled “Modular graft component junctions” is directed towards an endovascular graft having an attachment frame connection mechanism that allows placement of the main body component in vasculature in combination with a limb component or with limb components. The limb component is attached to the limb portion of the main body component by a frame or self-expanding stent at the proximal or superior end of the limb component. The frame of self-expanding stent is either inside the limb component or external the limb component with graft material folded over it. The limb can be manufactured with hooks already through its graft. When the proximal end of the limb component is inserted and deployed within the distal end of the limb support portion of the main body component, radially extending components in the form of hooks or barbs penetrate the graft material of the limb support portion of the main body component to form a graft-to-graft bond. The hooks or barbs may be incorporated within the self-expanding stent.

U.S. Patent Application Publication No. 2009/0299387 to Navia, entitled “Method and apparatus for fluidly isolating a portion of a body lumen wall from flow through the body lumen” is directed towards an apparatus for fluidly isolating a portion of a blood vessel wall from blood flow at an anastomosis site within the blood vessel. The apparatus includes an insertion catheter which has longitudinally spaced proximal and distal catheter ends and an operative lumen extending there between. The insertion catheter is configured for insertion into the blood vessel at an insertion location spaced apart from the anastomosis site. The apparatus also includes an isolation device which is attached to the distal catheter end and which includes a concave working surface bounded by an isolation rim and a blood flow surface opposite the working surface. The isolation rim is configured to contact at least a portion of the blood vessel and is longitudinally aligned with, and radially spaced from, the anastomosis site. The blood flow surface is in contact with blood flow past the anastomosis site within the blood vessel when the isolation device engages the blood vessel wall. The apparatus further includes a retention means which is attached to the isolation device and is in fluid communication with the operative lumen. The retention means is configured to exert force on the blood vessel wall to at least partially engage the blood vessel wall with the isolation device.

### SUMMARY OF THE PRESENT DISCLOSED TECHNIQUE

It is an object of the disclosed technique to provide a novel method and system for performing percutaneous coronary artery bypass graft surgery and other procedures involving an anastomosis of two vessels which overcomes the disadvantages of the prior art.

In accordance with the disclosed technique, there is thus provided an apparatus for performing bypass graft surgery on a patient, between a major blood vessel and an occluded vessel in the patient, the vessel being occluded by a lesion, including a blocking balloon, an endoscopic delivery device, a distal suture structure, a proximal suture structure and a delivery catheter. The blocking balloon is for isolating a portion of the major blood vessel. The endoscopic delivery device is for grasping a vessel graft and for navigating the vessel graft to the major blood vessel and the occluded vessel. The distal suture structure is positioned within a distal end of the vessel graft and is for suturing the vessel graft with the occluded vessel at a position distal to the lesion. The proximal suture structure is positioned within a proximal end of the vessel graft and is for suturing the vessel graft with the major blood vessel. The delivery catheter is positioned within the vessel graft and is for holding and releasing the distal suture structure and the proximal suturing structure.

According to another aspect of the disclosed technique, there is thus provided an apparatus for suturing a vessel graft to a vessel, including a stitching crown, a balloon catheter, a tube and a plurality of holding cones. The tube is coupled with the balloon catheter and a first one of the plurality of holding cones is coupled with a first end of the tube and a second one of the plurality of holding cones is coupled with a second end of the tube. The tube is for inflating and deflating the balloon catheter and the plurality of holding cones is for holding the stitching crown. The stitching crown includes a distal suture portion and a proximal suture portion, the distal suture portion being coupled with the proximal suture portion. The stitching crown is positioned around the balloon catheter and the distal suture structure and the proximal suture structure can be positioned in a closed state and in an open state.

According to a further aspect of the disclosed technique, there is thus provided an apparatus for suturing a vessel graft to a vessel including a distal suture portion and a proximal suture portion. The distal suture portion is coupled with the proximal suture portion. The distal suture portion includes a plurality of distal thorns and a plurality of distal jags. The proximal suture portion includes a plurality of proximal thorns and a plurality of proximal jags. The apparatus is cylindrical in nature and the plurality of distal thorns and the plurality of proximal thorns can be positioned in a closed state and in an open state.

According to another aspect of the disclosed technique, there is thus provided an apparatus for delivering a vessel graft between a major vessel and an occluded vessel in a patient, including a distal suture structure, a proximal suture structure and a delivery catheter. The distal suture structure is positioned within a distal end of the vessel graft. The proximal suture structure is positioned within a proximal end of the vessel graft. The delivery catheter is placed within the distal suture structure and the proximal suture structure. The distal suture structure is for suturing the vessel graft with the occluded vessel and is hollow. The proximal suture structure is for suturing the vessel graft with the major vessel and is hollow. The delivery catheter is for adjusting a position of the distal suture structure and the proximal suture structure within the vessel graft and is adjustable in length. The delivery catheter includes an outer catheter shaft having a first diameter and an inner catheter shaft having a second diameter. The second diameter is smaller than the first diameter and the inner catheter shaft and the outer catheter shaft can be adjusted relative to one another thereby adjusting a length of the delivery catheter. The distal suture structure and the proximal suture structure each have a diameter smaller than a diameter of the vessel graft and the delivery catheter is hollow and has a diameter smaller than a diameter of the distal suture structure and the proximal suture structure.

According to a further aspect of the disclosed technique, there is thus provided an apparatus for enabling access to a lumen in a body via a vessel in the body without impeding a flow of blood in the vessel including an inflatable hollow cylindrical portion, a first tube and a second tube. The second tube is coupled with the inflatable hollow cylindrical portion. The inflatable hollow cylindrical portion is for generating a blood-free area in the vessel. The first tube is for enabling access to the blood-free area and the second tube is for inflating and deflating the inflatable hollow cylindrical portion. The inflatable hollow cylindrical portion includes a first opening and a second opening. The first tube is coupled with the first opening. The first opening is positioned in the inflatable hollow cylindrical portion in a direction substantially perpendicular to the flow of blood. The second opening is positioned in the inflatable hollow cylindrical portion in a direction substantially parallel to the flow of blood, thereby enabling the flow of blood.

According to another aspect of the disclosed technique, there is thus provided an apparatus for enabling access to a lumen in a body via a vessel in the body without impeding a flow of matter in the vessel including an inflatable ring portion, an inflatable arch portion, a first tube and a second tube. The inflatable arch portion and the second tube are coupled with the inflatable ring portion. The inflatable ring portion is for generating a matter-free area in the vessel and the inflatable arch portion is for forming a first opening. The first tube is for enabling access to the matter-free area and the second tube is for inflating and deflating the inflatable ring portion and the inflatable arch portion. The inflatable ring portion includes a second opening. The first tube is coupled with the second opening. The second opening is positioned in the inflatable ring portion in a direction substantially perpendicular to the flow of matter and the first opening is formed in a direction substantially parallel to the flow of matter, thereby enabling the flow of matter.

According to a further aspect of the disclosed technique, there is thus provided an apparatus for enabling access to a lumen in a body via a vessel in the body without impeding a flow of matter in the vessel including an inflatable hollow cylindrical portion, a first tube and a second tube. The second tube is coupled with the inflatable hollow cylindrical portion. The inflatable hollow cylindrical portion is for generating a matter-free area in the vessel. The first tube is for enabling access to the matter-free area and the second tube is for inflating and deflating the inflatable hollow cylindrical portion. The inflatable hollow cylindrical portion includes a first opening and a second opening. The first tube is coupled with the first opening. The first opening is positioned in the inflatable hollow cylindrical portion in a direction substantially perpendicular to the flow of matter. The second opening is positioned in the inflatable hollow cylindrical portion in a direction substantially parallel to the flow of matter, thereby enabling the flow of matter.

According to another aspect of the disclosed technique, there is thus provided a balloon catheter for strengthening an anastomosis between a vessel graft to a vessel including an inner balloon, an outer balloon, an inner tube, an outer tube and a stitching crown. The inner tube is coupled with the inner balloon and the outer tube is coupled with the outer balloon. The outer balloon is positioned over the inner balloon and the stitching crown is positioned over the outer balloon. The inner tube is for inflating and deflating the inner balloon and the outer tube is for inflating and deflating the outer balloon. The stitching crown includes a distal suture portion and a proximal suture portion. The proximal suture portion is coupled with the distal suture portion. The outer balloon includes a plurality of release holes. The distal suture structure and the proximal suture structure can be positioned in a closed state and in an open state.

According to a further aspect of the disclosed technique, there is thus provided a method for constructing a stitching crown for suturing a vessel graft to a vessel, the stitching crown including a central portion and a plurality of jags. The method includes the procedures of cutting out the central portion and the plurality of jags from a sheet metal as a single element, rolling the single element into a circular form and coupling a first side of the central portion to a second side of the central portion.

According to another aspect of the disclosed technique, there is thus provided a method for constructing a stitching crown for suturing a vessel graft to a vessel, the stitching crown including a central portion and a plurality of jags. The method includes the procedures of placing a shape memory alloy into a memory shape, unfurling the shape memory alloy into a flat sheet, cutting out the central portion and the plurality of jags from the flat sheet as a single element and returning the shape memory alloy to the memory shape. The memory shape is circular.

According to a further aspect of the disclosed technique, there is thus provided a method for constructing a stitching crown for suturing a vessel graft to a vessel, the stitching crown including a central portion and a plurality of jags. The method includes the procedures of cutting out the central portion from a first material, cutting out the plurality of jags from a second material and coupling the central portion to the plurality of jags.

According to another aspect of the disclosed technique, there is thus provided an apparatus for suturing a vessel graft to a vessel, including a plurality of thorns, a plurality of jags and a connector. The connector is coupled with the plurality of thorns and the plurality of jags. The connector has a substantially circular shape and a diameter smaller than a diameter of the vessel graft. The plurality of thorns and the plurality of jags can be placed in an open state and a closed state.

According to a further aspect of the disclosed technique, there is thus provided an apparatus for delivering a vessel graft percutaneously and endoscopically to a desired location within a lumen of an individual including a modified endoscope and a vessel graft holder. The vessel graft holder is coupled with the modified endoscope. The modified endoscope is for maneuvering percutaneously and endoscopically within the individual and the vessel grafter holder is for holding the vessel graft. The modified endoscope includes a plurality of flexible backbone sections, a working channel, an image viewer, at least one light source, a plurality of suction holes and a suction tube. Each one of the plurality of flexible backbone sections is coupled with another one of the plurality of flexible backbone sections. The working channel extends along a length of the plurality of flexible backbone sections. The image viewer is positioned at a distal end of the modified endoscope substantially above the working channel. The at least one light source is positioned substantially adjacent to the working channel and the image viewer. The plurality of suction holes is positioned at a distal end of the modified endoscope on an outer surface of the modified endoscope. The suction tube is coupled with the plurality of suction holes. The plurality of flexible backbone sections is for navigating the modified endoscope within the individual. The working channel is for injecting and draining at least one substance. The image viewer is for enabling at least one image to be viewed. The at least one light source is for lighting a path of the modified endoscope within the individual. The plurality of suction holes is for temporarily affixing the modified endoscope to the desired location in the individual. The suction tube is for generating a vacuum at the plurality of suction holes.

According to another aspect of the disclosed technique, there is thus provided a method for performing an anastomosis of a vessel graft in an individual between a first vessel and a second vessel, using a suturing apparatus, an endoscopic delivery device and a blocking balloon. The method includes the procedures of imaging the first vessel and the second vessel, performing an incision in the individual to a major vessel, inserting an introducer in the incision for generating an entry-point into the major vessel and percutaneously inserting the blocking balloon via the entry-point into the major vessel. The procedure of imaging is for determining a first suturing point and a second suturing point of the vessel graft and a path from the first suturing point to the second suturing point, to respectively the first vessel and the second vessel. The method also includes the procedures of maneuvering the blocking balloon to the first suturing point in the first vessel, inflating the blocking balloon at the first suturing point, inserting a guiding catheter into the individual and maneuvering the guiding catheter to a vicinity of the second suturing point. The procedure of inflating the blocking balloon is for generating a matter-free working space around the first suturing point, the matter-free working space being coupled with a first tube of the blocking balloon. The method further includes the procedures of loading the suturing apparatus into the vessel graft outside the individual, loading the vessel graft into the endoscopic delivery device outside the individual, inserting the endoscopic delivery device into the first tube of the blocking balloon and maneuvering the endoscopic delivery device to the matter-free working space. The method also includes the procedures of advancing a puncturing wire to the matter-free working space, puncturing the first vessel at the first suturing point using the puncturing wire, removing the puncturing wire from the matter-free working space and endoscopically advancing the endoscopic delivery device through the punctured first suturing point. The method further includes the procedures of navigating the endoscopic delivery device to the second suturing point, generating suction at a distal end of the endoscopic delivery device using a plurality of suction holes positioned on the distal end of the endoscopic delivery device, temporarily affixing the distal end of the endoscopic delivery device to the vicinity of the second suturing point using the generated suction and puncturing the second vessel at the second suturing point. The method also includes the procedures of releasing a distal end of the vessel graft from the endoscopic delivery device, advancing the distal end of the vessel graft into the second suturing point, suturing the distal end of the vessel graft to the second suturing point using a distal suture structure in the suturing apparatus and releasing a proximal end of the vessel graft from the endoscopic delivery device. The method further includes the procedures of retracting the endoscopic delivery device into the first suturing point, pulling the proximal end of the vessel graft into the first suturing point and suturing the proximal end of the vessel graft to the first suturing point using a proximal suture structure in the suturing apparatus.

According to a further aspect of the disclosed technique, there is thus provided a method for performing at least one anastomosis of at least one vessel graft in an individual between at least one first vessel and at least one second vessel, using at least one suturing apparatus, at least one endoscopic delivery device and a blocking balloon. The method includes the procedures of imaging the at least one first vessel and the at least one second vessel, performing an incision in the individual to a major vessel, inserting an introducer in the incision for generating an entry-point into the major vessel and percutaneously inserting the blocking balloon via the entry-point into the major vessel. The procedure of imaging is for determining at least one first suturing point and at least one second suturing point of the at least one vessel graft and at least one path from the at least one first suturing point to the at least one second suturing point, to respectively the at least one first vessel and the at least one second vessel. The method also includes the procedures of maneuvering the blocking balloon to the at least one first suturing point in the at least one first vessel, inflating the blocking balloon at the at least one first suturing point, loading the at least one suturing apparatus into the at least one vessel graft outside the individual and loading the at least one vessel graft into the at least one endoscopic delivery device outside the individual. The procedure of inflating the blocking balloon is for generating a matter-free working space around the at least one first suturing point, the matter-free working space being coupled with a first tube of the blocking balloon. The method further includes the procedures of inserting the at least one endoscopic delivery device into the first tube of the blocking balloon, maneuvering the at least one endoscopic delivery device to the matter-free working space, advancing a puncturing wire to the matter-free working space and puncturing the at least one first vessel at the at least one first suturing point using the puncturing wire. The method also includes the procedures of removing the puncturing wire from the matter-free working space, endoscopically advancing the at least one endoscopic delivery device through the punctured at least one first suturing point, navigating the at least one endoscopic delivery device to the at least one second suturing point and generating suction at a distal end of the at least one endoscopic delivery device using a plurality of suction holes positioned on the distal end of the at least one endoscopic delivery device. The method further includes the procedures of temporarily affixing the distal end of the at least one endoscopic delivery device to a vicinity of the at least one second suturing point using the generated suction, puncturing the at least one second vessel at the at least one second suturing point, releasing a distal end of the at least one vessel graft from the at least one endoscopic delivery device and advancing the distal end of the at least one vessel graft into the at least one second suturing point. The method also includes the procedures of suturing the distal end of the at least one vessel graft to the at least one second suturing point using a distal suture structure in the at least one suturing apparatus, releasing a proximal end of the at least one vessel graft from the at least one endoscopic delivery device, retracting the at least one endoscopic delivery device into the at least one first suturing point and pulling the proximal end of the at least one vessel graft into the at least one first suturing point. The method further includes the procedures of suturing the proximal end of the at least one vessel graft to the at least one first suturing point using a proximal suture structure in the at least one suturing apparatus, slightly deflating the blocking balloon, navigating the blocking balloon to another at least one first suturing point in the at least one first vessel and inflating the blocking balloon at the another at least one first suturing point. The procedure of inflating the blocking balloon at the another at least one first suturing point is for generating another matter-free working space around the another at least one first suturing point. The method also includes the procedure of repeating the method at least once at the another at least one first suturing point starting from the procedure of inserting another one of the at least one endoscopic delivery device into the first tube of the blocking balloon.

According to another aspect of the disclosed technique, there is thus provided a method for performing an anastomosis of a vessel graft in an individual between a first vessel and a second vessel, using a suturing apparatus, an endoscopic delivery device and a blocking balloon, the endoscopic device including a modified endoscope and a vessel graft holder. The method includes the procedures of imaging the first vessel and the second vessel, performing an incision in the individual to a major vessel, inserting an introducer in the incision for generating an entry-point into the major vessel and percutaneously inserting the blocking balloon via the entry-point into the major vessel. The procedure of imaging is for determining a first suturing point and a second suturing point of the vessel graft and a path from the first suturing point to the second suturing point, to respectively the first vessel and the second vessel. The method also includes the procedures of maneuvering the blocking balloon to the first suturing point in the first vessel, inflating the blocking balloon at the first suturing point, inserting a guiding catheter into the individual and maneuvering the guiding catheter to a vicinity of the second suturing point. The procedure of inflating the blocking balloon is for generating a matter-free working space around the first suturing point, the matter-free working space being coupled with a first tube of the blocking balloon. The method further includes the procedures of loading the suturing apparatus into the vessel graft outside the individual, loading the vessel graft into the vessel graft holder outside the individual, inserting the modified endoscope into the first tube of the blocking balloon and maneuvering the modified endoscope to the matter-free working space. The method also includes the procedures of advancing a puncturing wire to the matter-free working space, puncturing the first vessel at the first suturing point using the puncturing wire, removing the puncturing wire from the matter-free working space and endoscopically advancing the modified endoscope through the punctured first suturing point. The method further includes the procedures of navigating the modified endoscope to the second suturing point, generating suction at a distal end of the modified endoscope using a plurality of suction holes positioned on the distal end of the modified endoscope, temporarily affixing the distal end of the modified endoscope to the vicinity of the second suturing point using the generated suction and puncturing the second vessel at the second suturing point. The method also includes the procedures of retracting the modified endoscope from the individual, coupling the vessel graft holder to the modified endoscope, re-inserting the modified endoscope coupled with the vessel graft holder into the individual to the second suturing point and releasing a distal end of the vessel graft from the vessel graft holder. The method further includes the procedures of advancing the distal end of the vessel graft into the second suturing point, suturing the distal end of the vessel graft to the second suturing point using a distal suture structure in the suturing apparatus, releasing a proximal end of the vessel graft from the vessel graft holder and retracting the endoscopic delivery device into the first suturing point. The method also includes the procedures of pulling the proximal end of the vessel graft into the first suturing point and suturing the proximal end of the vessel graft to the first suturing point using a proximal suture structure in the suturing apparatus.

According to a further aspect of the disclosed technique, there is thus provided a method for performing an anastomosis of a vessel graft in an individual between a first vessel and a second vessel, using a suturing apparatus, an endoscopic delivery device and a blocking balloon, the endoscopic device including a modified endoscope and a vessel graft holder. The method includes the procedures of imaging the first vessel and the second vessel, performing an incision in the individual to a major vessel, inserting an introducer in the incision for generating an entry-point into the major vessel and percutaneously inserting the blocking balloon via the entry-point into the major vessel. The procedure of imaging is for determining a first suturing point and a second suturing point of the vessel graft and a path from the first suturing point to the second suturing point, to respectively the first vessel and the second vessel. The method also includes the procedures of maneuvering the blocking balloon to the first suturing point in the first vessel, inflating the blocking balloon at the first suturing point, inserting a guiding catheter into the individual and maneuvering the guiding catheter to a vicinity of the second suturing point. The procedure of inflating the blocking balloon is for generating a matter-free working space around the first suturing point, the matter-free working space being coupled with a first tube of the blocking balloon. The method further includes the procedures of loading the suturing apparatus into the vessel graft outside the individual, loading the vessel graft into the vessel graft holder outside the individual, inserting the modified endoscope into the first tube of the blocking balloon and maneuvering the modified endoscope to the matter-free working space. The method also includes the procedures of advancing a puncturing wire to the matter-free working space, puncturing the first vessel at the first suturing point using the puncturing wire, removing the puncturing wire from the matter-free working space and endoscopically advancing the modified endoscope through the punctured first suturing point. The method further includes the procedures of navigating the modified endoscope to the second suturing point, generating suction at a distal end of the modified endoscope using a plurality of suction holes positioned on the distal end of the modified endoscope, temporarily affixing the distal end of the modified endoscope to the vicinity of the second suturing point using the generated suction and puncturing the second vessel at the second suturing point. The method also includes the procedures of retracting the modified endoscope from the individual, inserting the vessel graft holder into the individual to the second suturing point, releasing a distal end of the vessel graft from the vessel graft holder and advancing the distal end of the vessel graft into the second suturing point. The method further includes the procedures of suturing the distal end of the vessel graft to the second suturing point using a distal suture structure in the suturing apparatus, releasing a proximal end of the vessel graft from the vessel graft holder, retracting the vessel graft holder into the first suturing point, pulling the proximal end of the vessel graft into the first suturing point and suturing the proximal end of the vessel graft to the first suturing point using a proximal suture structure in the suturing apparatus.

According to another aspect of the disclosed technique, there is thus provided a method for performing an anastomosis of a vessel graft in an individual between a vessel and a second vessel, using a suturing apparatus, an endoscopic delivery device and a blocking balloon, the endoscopic device including a modified endoscope and a vessel graft holder. The method includes the procedures of imaging the first vessel and the second vessel, performing an incision in the individual to a major vessel, inserting an introducer in the incision for generating an entry-point into the major vessel and percutaneously inserting the blocking balloon via the entry-point into the major vessel. The procedure of imaging is for determining a first suturing point and a second suturing point of the vessel graft and a path from the first suturing point to the second suturing point, to respectively the first vessel and the second vessel. The method also includes the procedures of maneuvering the blocking balloon to the first suturing point in the first vessel, inflating the blocking balloon at the first suturing point, inserting a guiding catheter into the individual and maneuvering the guiding catheter to a vicinity of the second suturing point. The procedure of inflating the blocking balloon is for generating a matter-free working space around the first suturing point, the matter-free working space being coupled with a first tube of the blocking balloon. The method further includes the procedures of loading the suturing apparatus into the vessel graft outside the individual, loading the vessel graft into the vessel graft holder outside the individual, inserting the modified endoscope into the first tube of the blocking balloon and maneuvering the modified endoscope to the matter-free working space. The method also includes the procedures of advancing a puncturing wire to the matter-free working space, puncturing the first vessel at the first suturing point using the puncturing wire, removing the puncturing wire from the matter-free working space and endoscopically advancing the modified endoscope through the punctured first suturing point. The method further includes the procedures of navigating the modified endoscope to the second suturing point, generating suction at a distal end of the modified endoscope using a plurality of suction holes positioned on the distal end of the modified endoscope, temporarily affixing the distal end of the modified endoscope to the vicinity of the second suturing point using the generated suction and puncturing the second vessel at the second suturing point. The method also includes the procedures of inserting the vessel graft holder through a conduit coupled with the modified endoscope into the individual to the second suturing point, releasing a distal end of the vessel graft from the vessel graft holder, advancing the distal end of the vessel graft into the second suturing point and suturing the distal end of the vessel graft to the second suturing point using a distal suture structure in the suturing apparatus. The method further includes the procedures of releasing a proximal end of the vessel graft from the vessel graft holder, retracting the endoscopic delivery device into the first suturing point, pulling the proximal end of the vessel graft into the first suturing point and suturing the proximal end of the vessel graft to the first suturing point using a proximal suture structure in the suturing apparatus.

### BRIEF DESCRIPTION OF THE DRAWINGS

The disclosed technique will be understood and appreciated more fully from the following detailed description taken in conjunction with the drawings in which:

FIG. 1A is a schematic illustration of a human heart with a blocked coronary artery, as known in the prior art;

FIG. 1B is a schematic illustration of a human heart with a blocked coronary artery, constructed and operative in accordance with an embodiment of the disclosed technique;

FIG. 2A is a schematic illustration of a heart bypass surgery suturing apparatus in a disassembled form, constructed and operative in accordance with another embodiment of the disclosed technique;

FIG. 2B is a schematic illustration of the heart bypass surgery suturing apparatus of FIG. 2A is an assembled form, constructed and operative in accordance with a further embodiment of the disclosed technique;

FIG. 3A is a schematic illustration of additional apparatuses to be used with the heart bypass surgery suturing apparatus of FIG. 2A, constructed and operative in accordance with another embodiment of the disclosed technique;

FIG. 3B is a schematic illustration of the additional apparatuses of FIG. 3A being used with the heart bypass surgery suturing apparatus of FIG. 2A, constructed and operative in accordance with a further embodiment of the disclosed technique;

FIG. 4A is a perspective schematic illustration of an endoscopic delivery device, constructed and operative in accordance with another embodiment of the disclosed technique;

FIG. 4B is a schematic illustration of the movement capabilities of the endoscopic delivery device of FIG. 4A, constructed and operative in accordance with a further embodiment of the disclosed technique;

FIG. 4C is another perspective schematic illustration of the endoscopic delivery device of FIG. 4A, constructed and operative in accordance with another embodiment of the disclosed technique;

FIG. 4D is a top orthogonal schematic illustration of different rib arrangements of the endoscopic delivery device of FIG. 4A, constructed and operative in accordance with a further embodiment of the disclosed technique;

FIG. 4E is a cross-sectional schematic illustration of the ribs of the endoscopic delivery device of FIG. 4A in an open and closed state, constructed and operative in accordance with another embodiment of the disclosed technique;

FIG. 4F is a cross-sectional schematic illustration of different embodiments of the blood vessel graft holder of the endoscopic delivery device of FIG. 4A, constructed and operative in accordance with a further embodiment of the disclosed technique;

FIG. 5A is a perspective schematic illustration of a blocking balloon, constructed and operative in accordance with another embodiment of the disclosed technique;

FIG. 5B is a perspective schematic illustration of the blocking balloon of FIG. 5A placed within a blood vessel, constructed and operative in accordance with a further embodiment of the disclosed technique;

FIG. 5C is another perspective schematic illustration of the blocking balloon of FIG. 5A, constructed and operative in accordance with another embodiment of the disclosed technique;

FIG. 5D is a perspective schematic illustration of another blocking balloon, constructed and operative in accordance with a further embodiment of the disclosed technique;

FIG. 5E is a perspective schematic illustration of a first placement of radiopaque markers on the blocking balloon of FIG. 5A, constructed and operative in accordance with another embodiment of the disclosed technique;

FIG. 5F is a perspective schematic illustration of a second placement of radiopaque markers on the blocking balloon of FIG. 5A, constructed and operative in accordance with a further embodiment of the disclosed technique;

FIGS. 6A-6H are schematic illustrations of the use of the heart bypass surgery suturing apparatus of FIGS. 2A-3B, the endoscopic delivery device of FIGS. 4A-4F and the blocking balloon of FIGS. 5A-5F in a TABG surgery of the disclosed technique, constructed and operative in accordance with another embodiment of the disclosed technique;

FIGS. 7A-7B are schematic illustrations of a suture structure used to suture a blood vessel graft to a blood vessel, constructed and operative in accordance with a further embodiment of the disclosed technique;

FIG. 7C is a perspective schematic illustration of another suture structure used to suture a blood vessel graft to a blood vessel, constructed and operative in accordance with another embodiment of the disclosed technique;

FIG. 7D shows orthogonal projections of further suture structures used to suture a blood vessel graft to a blood vessel, constructed and operative in accordance with a further embodiment of the disclosed technique;

FIGS. 8A-8C are schematic illustrations showing a first mechanism for opening the suture structure of FIGS. 7A-7D, constructed and operative in accordance with another embodiment of the disclosed technique;

FIG. 9A is a schematic illustration showing a second mechanism for opening the suture structure of FIGS. 7A-7D, constructed and operative in accordance with a further embodiment of the disclosed technique;

FIG. 9B is a schematic illustration showing a third mechanism for opening the suture structure of FIGS. 7A-7D, constructed and operative in accordance with another embodiment of the disclosed technique;

FIGS. 9C-9D are schematic illustrations showing a fourth mechanism for opening the suture structure of FIGS. 7A-7D, constructed and operative in accordance with a further embodiment of the disclosed technique;

FIG. 10A is a schematic illustration showing the transition of a suture structure from a closed state to an open state, constructed and operative in accordance with another embodiment of the disclosed technique;

FIGS. 10B-10D are schematic illustrations showing a plurality of pre-defined suturing shapes of the suture structure of FIG. 10A, constructed and operative in accordance with a further embodiment of the disclosed technique;

FIG. 11A is a schematic illustration of a stitching crown suture structure, constructed and operative in accordance with another embodiment of the disclosed technique;

FIG. 11B is a schematic illustration of the stitching crown suture structure of FIG. 11A used to suture two vessels, constructed and operative in accordance with a further embodiment of the disclosed technique;

FIG. 12 is a schematic illustration of a distal suture structure, constructed and operative in accordance with another embodiment of the disclosed technique;

FIGS. 13A-13D are schematic illustrations of the distal suture structure of FIG. 12 used in a TABG surgery of the disclosed technique, constructed and operative in accordance with a further embodiment of the disclosed technique;

FIGS. 14A-14B are perspective schematic illustrations of a proximal suture structure, constructed and operative in accordance with another embodiment of the disclosed technique;

FIG. 14C is a side orthogonal schematic illustration of different embodiments of the proximal suture structure of FIG. 14A, constructed and operative in accordance with a further embodiment of the disclosed technique;

FIG. 14D is another schematic illustration of the proximal suture structure of FIG. 14A, constructed and operative in accordance with another embodiment of the disclosed technique;

FIG. 15 is a schematic illustration of a heart bypass surgery suturing apparatus fully deployed in a patient, constructed and operative in accordance with a further embodiment of the disclosed technique;

FIG. 16A is a top orthogonal schematic illustration of a channel for opening and closing the ribs of an endoscopic delivery device, constructed and operative in accordance with another embodiment of the disclosed technique;

FIG. 16B is a top and side orthogonal schematic illustration of the channel of FIG. 16A, showing the opening and closing of the ribs of an endoscopic delivery device using the channel, constructed and operative in accordance with a further embodiment of the disclosed technique;

FIG. 17 is a front orthogonal schematic illustration of a heart bypass surgery suturing apparatus including an endoscopic delivery device employing the channel of FIGS. 16A-16B, a blocking balloon, a suture structure and a blood vessel graft, constructed and operative in accordance with another embodiment of the disclosed technique;

FIG. 18A is a schematic illustration of a further blocking balloon in a deflated state, constructed and operative in accordance with a further embodiment of the disclosed technique;

FIG. 18B is a schematic illustration of the blocking balloon of FIG. 18A an in inflated state, constructed and operative in accordance with another embodiment of the disclosed technique;

FIG. 19A is a schematic illustration of a suture structure with a release apparatus for deploying a stitching crown showing the deployment of the stitching crown, constructed and operative in accordance with a further embodiment of the disclosed technique;

FIG. 19B is a schematic illustration of the suture structure of FIG. 19A with a release apparatus for deploying a stitching crown showing the deployment of the stitching crown with a blood vessel graft and a blood vessel, constructed and operative in accordance with another embodiment of the disclosed technique;

FIG. 20A is a first schematic illustration of a manufacturing method of a suturing structure, constructed and operative in accordance with a further embodiment of the disclosed technique;

FIGS. 20B-20C are second and third schematic illustrations of the manufacturing method of the suturing structure of FIG. 20A, constructed and operative in accordance with another embodiment of the disclosed technique;

FIG. 20D is a fourth schematic illustration of the manufacturing method of the suturing structure of FIG. 20A, constructed and operative in accordance with a further embodiment of the disclosed technique;

FIGS. 21A-21C are schematic illustrations of a suture structure enabling the deployment of a biological glue, constructed and operative in accordance with another embodiment of the disclosed technique;

FIG. 22A is a schematic illustration of another endoscopic delivery device, constructed and operative in accordance with a further embodiment of the disclosed technique; and

FIG. 22B is a schematic illustration of a further endoscopic delivery device, constructed and operative in accordance with another embodiment of the disclosed technique.

### DETAILED DESCRIPTION OF THE EMBODIMENTS

The disclosed technique overcomes the disadvantages of the prior art by providing a novel system and method for performing percutaneous coronary artery bypass graft surgery. The disclosed technique is low cost, does not require a large surgical team and can be performed without patient sedation. In addition, the disclosed technique can be performed off-pump, involves minimally invasive surgery (i.e., no open chest surgery) and can be performed in a substantially short amount of time, for example within the range of an hour. Furthermore, the general principles and apparatuses of the disclosed technique are similar to the general principles and apparatuses used in bypass heart surgery according to the prior art, thereby enabling surgeons and medical practitioners to use the apparatuses of the disclosed technique without having to undergo an extensive training period. The general principles of the disclosed technique are similar to the prior art in that an alternative route for blood to flow to the heart is created by coupling a blood vessel graft from the aorta to a point on the coronary arteries which is distal to a blocked coronary artery. It is noted that the disclosed technique can be used to perform heart bypass surgery by suturing a blood vessel graft between an aorta and a coronary artery of a heart having a lesion in one or more of its coronary arteries.

According to the disclosed technique, a blood vessel graft is inserted and navigated to the aorta of an individual via the femoral artery or the radial artery in a manner similar to how PCI surgery is performed. The blood vessel graft is navigated to the aorta using a novel endoscopic delivery device and a novel blocking balloon according to the disclosed technique. According to the disclosed technique, the entire procedure of coupling the blood vessel graft to the heart (i.e., implanting the blood vessel graft), thereby creating an alternate route for blood to flow to the heart, is performed through the aorta. The blood vessel graft is sutured to the heart using novel suturing devices. The disclosed technique enables percutaneous coronary artery bypass graft surgery to be performed significantly faster than prior art systems and methods, can be performed off-pump on a beating heart (i.e., without the use of a heart-lung machine) and can shorten the amount of time a patient undergoing such a procedure needs to spend in intensive care after surgery. In addition, the disclosed technique enables the overall hospitalization time of a patient undergoing coronary artery bypass graft surgery to be shortened while accelerating the patient's recovery and healing due to the minimally invasive nature of the surgery performed according to and with the systems of the disclosed technique.

Throughout the disclosed technique the terms “user,” “surgeon,” “operator” and “medical practitioner” are used interchangeably to refer to an individual using the disclosed technique to perform off-pump, percutaneous coronary artery bypass graft surgery. The terms “individual,” “person” and “patient” are used interchangeably to refer to the individual on whom the coronary artery bypass graft surgery of the disclosed technique is being performed on.

In general, the disclosed technique is performed as follows on a patient diagnosed with a lesion in one or more of his coronary arteries. The disclosed technique is elaborated on in the description of the figures that follow below. An incision is made in one of the major arteries of the body of the patient which couples with the aorta, such as the femoral artery or the radial artery. The novel blocking balloon of the disclosed technique is inserted via the incision and navigated to the aortic arch of the patient, where it is inflated. Due to its shape and design, the novel blocking balloon generates a sterile, blood-free working space on the inner side or wall of the aortic arch. A standard guiding catheter and guidewire are then inserted and navigated through the incision to a desired point either past the lesion in the heart or adjacent to, but not necessarily past, the lesion in the heart, as is done in standard PCI surgery. The tip or end of the guidewire is used as a target designator for marking a point beyond the lesion. In the working space of the novel blocking balloon, a puncture is then made in the aorta's wall while keeping the working space isolated from blood flowing in the aortic arch. A blood vessel graft is then fitted with the novel suturing devices of the disclosed technique, which include a distal suture structure and a proximal suture structure. The blood vessel graft with the fitted suturing devices is then placed in a blood vessel graft holder which is part of the novel endoscopic delivery device of the disclosed technique. The blood vessel graft holder includes a plurality of flexible ribs which can be opened and closed for holding and releasing the blood vessel graft. The novel endoscopic delivery device also includes a camera, at least one light source, a working channel and a plurality of suction holes. The novel endoscopic delivery device is also flexible and has the ability to be manipulated bilaterally.

The endoscopic delivery device with the blood vessel graft is then inserted via the working channel of the novel blocking balloon until the puncture in the aorta's wall. The endoscopic delivery device is then navigated through the puncture in the aorta's wall towards the pericardium of the heart, which is the sac of serous membrane which surrounds the heart and the roots of the major blood vessels which enter and exit the heart. The endoscopic delivery device is navigated towards the designated target which as mentioned above is a point beyond the lesion in one of the coronary arteries, within the pericardium using both real-time fluoroscopy as well as real-time imaging based on images received from the camera in the endoscopic delivery device. The endoscopic delivery device is navigated towards the tip of the guidewire initially inserted into the heart to a point past the lesion in the heart. The tip of the guidewire substantially represents a coupling point to which the blood vessel graft will be coupled with the coronary artery which is occluded, at a point on the coronary artery which is distal to the lesion. Once the coupling point is determined, another puncture is made, this time in the coronary artery to be bypassed. This other puncture is distal to the lesion in the coronary artery and can be made by a wire fitted for such a purpose according to the disclosed technique.

The novel suturing devices of the disclosed technique, coupled with a guidewire, are then used to push the distal end of the blood vessel graft into the puncture made in the coronary artery. Once the distal end of the blood vessel graft is properly placed within the coronary artery, the novel distal suture structure of the disclosed technique is used to perform a first anastomosis to couple the distal end of the blood vessel graft with the coronary artery. The endoscopic delivery device of the disclosed technique is then pulled back, thereby pulling the proximal end of the blood vessel graft towards the puncture made in the wall of the aortic arch. Once the proximal end of the blood vessel graft is properly placed within the aortic arch, the novel proximal suture structure of the disclosed technique is used to perform a second anastomosis to couple the proximal end of the blood vessel graft with the aortic arch. A contrast agent can then be injected, for example via the working channel of the endoscopic delivery device, to verify the flow of liquids via the bypass created by the blood vessel graft before the novel blocking balloon which created the blood-free working space is removed from the aorta. According to the disclosed technique, the above described procedure can be repeated for other blocked arteries, thus executing multiple bypasses while using the same blocking balloon. The above described procedure can be also performed in conjunction with a standard PCI surgical procedure being executed in an adjacent blocked artery. The disclosed technique thus provides for the hybrid capability of using a bypass graft and a stent in the same surgical procedure. Based on the novel apparatuses and methods of the disclosed technique, CABG surgery performed according to the disclosed technique can be referred to as transcatheter coronary artery bypass graft surgery, abbreviated herein either as TCABG or TABG.

It is also noted, as described below, that any of the procedures executed in the TABG surgery according to the disclosed technique which involve navigation and manipulation inside a patient can be performed manually by a medical practitioner or by a robotic manipulator controlled by a medical practitioner. Examples of known robotic manipulators which can be used with the disclosed technique include the C or Path® 200 system made by Corindus Vascular Robotics as well as the Sensei® X Robotic Catheter system by Hansen Medical, Inc.

In addition, it is also noted that even though the disclosed technique is described as it applies to performing a TABG surgery, the apparatuses of the disclosed technique as well as their methods of use can be applied to performing other procedures in an individual. For example, the novel blocking balloon of the disclosed technique can be used in medical procedures where access to a lumen, cavity or organ in an individual is achieved via a major blood vessel, a peripheral blood vessel, the trachea, the esophagus or the anus. In this respect, the novel blocking balloon allows a variety of matter within an individual to continue flowing while a surgical procedure is performed. As described below in great detail, the novel blocking balloon of the disclosed technique, when used in a major blood vessel or a peripheral blood vessel, enables blood to continue flowing while a medical procedure is executed. In addition, using the novel blocking balloon in the trachea enables air to continue flowing in and out of the respiratory system. Using the novel blocking balloon in the anus enables fecal matter to continue to flow, whereas using the novel blocking balloon in the esophagus enables food to continue to move and flow in the digestive tract.

In general, the disclosed technique can be used for performing an anastomosis between a first vessel and a second vessel, where the first vessel and the second vessel are coupled together via a vessel graft. It is noted that the second vessel may alternatively be an organ. The first and second vessels may be a major blood vessel, a vessel of the pulmonary system of the body such as the trachea or one of the bronchi, a vessel of the digestive system such as the esophagus or small intestines or a vessel of the excretory system such as the anus. The organ may be the heart, the lungs, the stomach, the pancreas, the liver, the bladder or any other major organ in the body. Whereas the disclosed technique is described in relation to an anastomosis between the aortic arch of a patient and a coronary artery on his heart, the disclosed technique can be applied to many other surgical procedures where a bypass route for matter in the body is to be created between a first vessel in the body and a second vessel in the body.

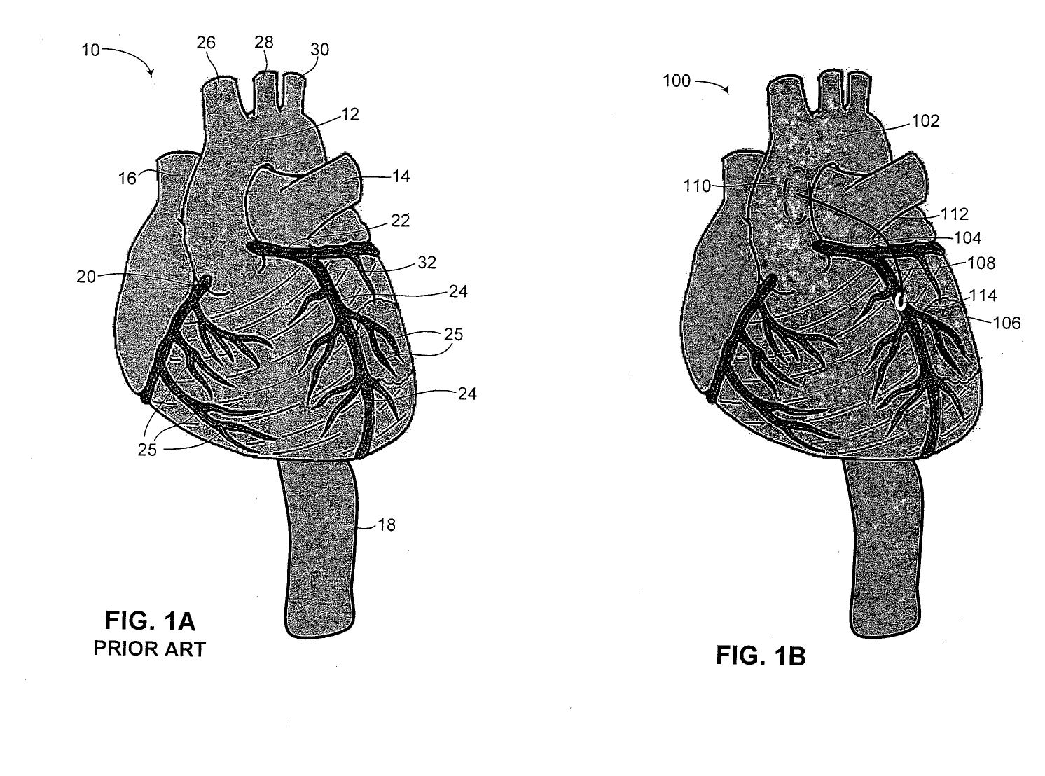

Reference is now made to FIG. 1B, which is a schematic illustration of a human heart with a blocked coronary artery, generally referenced 100, constructed and operative in accordance with an embodiment of the disclosed technique. Heart 100 is similar to heart 10 (FIG. 1A) and includes an aorta 102, a left main coronary artery 104 and a LAD coronary artery 106. Heart 100 includes a lesion 108, substantially occluding most of the blood flow from left main coronary artery 104 to LAD coronary artery 106 and to the network of smaller coronary arteries branching off of LAD coronary artery 106 (not labeled). Lesion 108 may be plaque material, calcium, fibrous material, fatty acids or any other buildup in the inner walls of the coronary blood vessels of heart 100. As described below in greater detail, according to the disclosed technique, a blood vessel graft (not shown) is inserted percutaneously into a major blood vessel of a patient (not shown), such as the descending aorta, and maneuvered to a location proximal to lesion 108, demarcated as a puncture location 110 (shown in FIG. 1B as a dotted circle). The blood vessel graft is mounted on a suturing device (not shown) and an endoscopic delivery device (not shown), both constructed according to the disclosed technique and described in detail below. The endoscopic delivery device and the suturing device are maneuvered with the blood vessel graft to puncture location 110, where a hole is made in the wall of aorta 102. The blood vessel graft is then maneuvered through the hole in puncture location 110, via a path 112 until a location distal to lesion 108, demarcated as an attachment location 114. Using the suture device of the disclosed technique, the blood vessel graft is sutured to both attachment location 114 and puncture location 110, thereby creating an alternative route for blood to flow and circulate from aorta 102 to LAD coronary artery 106 and the network of smaller coronary arteries which branch off from there.

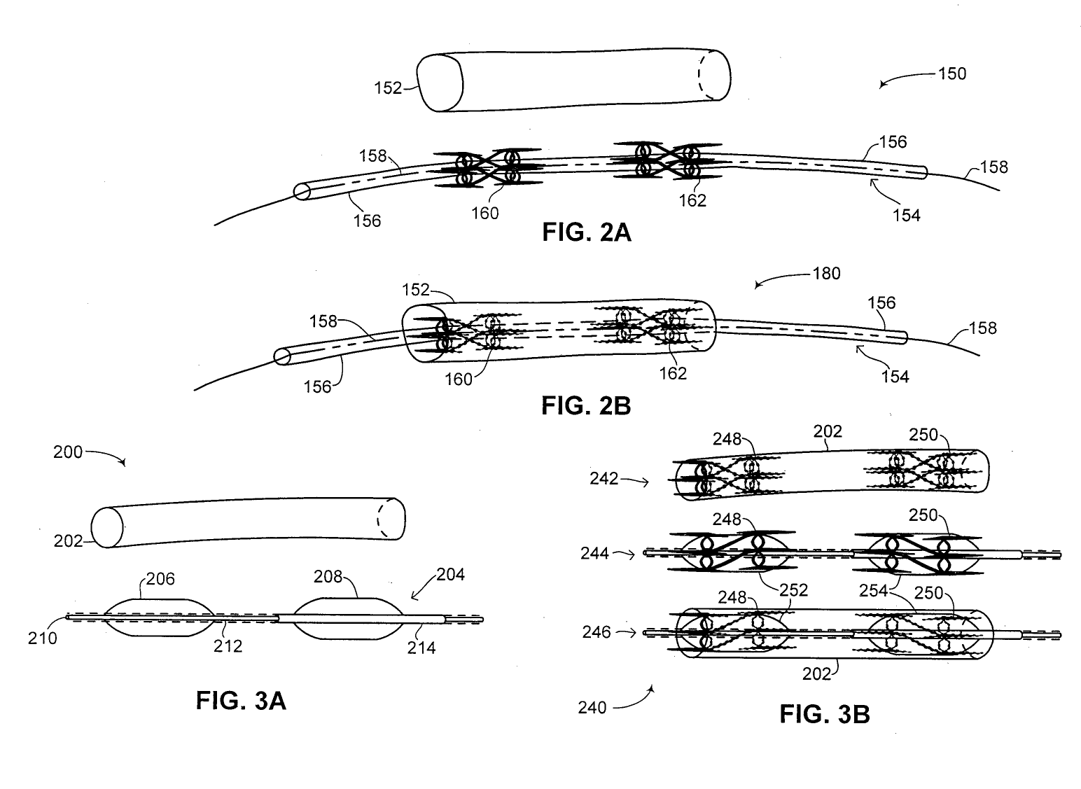

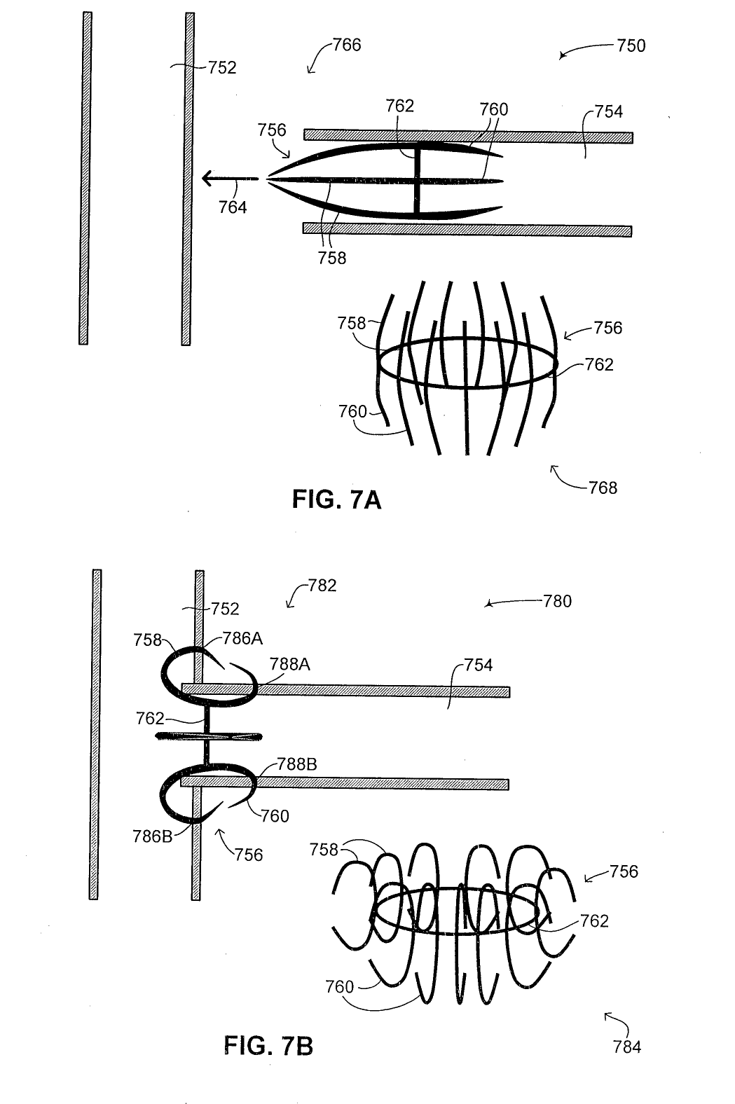

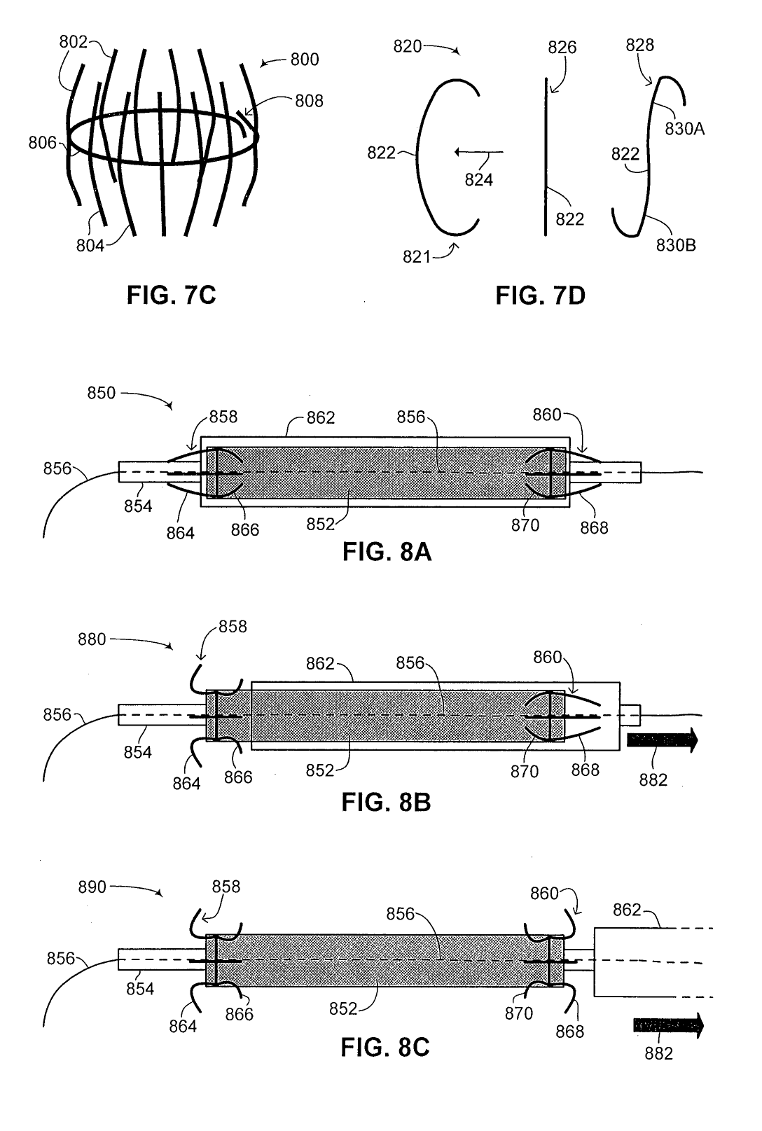

Reference is now made to FIG. 2A, which is a schematic illustration of a heart bypass surgery suturing apparatus in a disassembled form, generally referenced 150, constructed and operative in accordance with another embodiment of the disclosed technique. Heart bypass surgery suturing apparatus 150 includes a blood vessel graft 152 and a suturing device 154. Suturing device 154 includes a delivery catheter 156, a guidewire 158, a distal suture structure 160 and a proximal suture structure 162. Distal suture structure 160 and proximal suture structure 162 are made of either Nitinol, Flexinol®, stainless steel, nickel-free stainless steel, cobalt chrome, titanium, cobalt chromium molybdenum, a bio-absorbable material and the like, and are either self expandable or opened by inflating a balloon. Distal suture structure 160 and proximal suture structure 162 are described in greater detail below in FIGS. 7A-7D, 10A-10D and 11A-11B. Blood vessel graft 152 may be a natural vessel graft, such as a vessel graft harvested from a patient's leg (not shown). In such a case, the vessel graft may be harvested using standard surgical practices for harvesting vessel grafts. The vessel graft may also be harvested using an endoscopic harvesting tool, such as the VASOVIEW® 5 Endoscopic Vessel Harvesting System, from Guidant Corporation. The vessel graft may be a vein graft or an artery graft, for example an artery graft harvested from a patient's radial artery. It is also noted that if the blood vessel graft is a natural vessel graft then it may be covered with Dacron®, or surrounded by a metal fixture. Blood vessel graft 152 may also be an artificial vessel graft. In such a case, blood vessel graft 152 may be, for example, a Dacron® graft surrounded by a metal fixture or a metal mesh, similar to an abdominal aortic aneurysm graft except smaller in diameter. Blood vessel graft 152 may also be a biosynthetic vascular graft lined with a patient's own endothelial cells, such as the MultiGeneGraft available from the company MGVS. Blood vessel graft 152 may also be a vein graft covered by an extravascular prosthesis, made of metal or other suitable materials, such as the eSVS® Mesh from Kips Bay Medical, Inc. It is noted that blood vessel graft 152 may be any other artificial graft without limitations.

Delivery catheter 156 includes a hollow lumen, or working channel, through which guidewire 158 can be inserted. Distal suture structure 160 and proximal suture structure 162 are substantially cylindrical in shape and are hollow such that they can be inserted over delivery catheter 156. Delivery catheter 156 is adjustable in length to match the length of blood vessel graft 152. In addition, the positions of distal suture structure 160 and proximal suture structure 162 can be adjusted along delivery catheter 156 in order to be properly positioned vis-à-vis blood vessel graft 152, as shown in FIG. 2B.

Reference is now made to FIG. 2B, which is a schematic illustration of the heart bypass surgery suturing apparatus of FIG. 2A is an assembled form, generally referenced 180, constructed and operative in accordance with a further embodiment of the disclosed technique. Elements in FIG. 2B similar to elements in FIG. 2A are labeled using equivalent numbers. As shown in FIG. 2B, suturing device 154 has a diameter (not shown) small enough such that blood vessel graft 152 can be inserted over delivery catheter 156, distal suture structure 160 and proximal suture structure 162. As mentioned above, the length of delivery catheter 156 can be adjusted to match the length of blood vessel graft 152. Distal suture structure 160 and proximal suture structure 162 are positioned along delivery catheter 156 such that distal suture structure 160 is positioned at a distal end of blood vessel graft 152 and proximal suture structure 162 is positioned at a proximal end of blood vessel graft 152. As described below in FIGS. 3A and 3B, suturing device 154 may include additional apparatuses for adjusting and holding the positions of distal suture structure 160 and proximal suture structure 162 once blood vessel graft 152 has been placed over suturing device 154. Also, as shown in greater detail below in FIGS. 3A and 3B, delivery catheter 156 includes two balloon catheter shafts (not shown in FIGS. 2A and 2B). One balloon catheter shaft is larger in diameter than the other such that the larger diameter balloon catheter shaft can be slid over the smaller diameter balloon catheter shaft. The two shafts can be moved relative to one another thereby enabling the length of delivery catheter 156 to be adjusted. When blood vessel graft 152 is placed over delivery catheter 156, the two balloon catheter shafts are adjusted such that delivery catheter 156 is approximately the same length as blood vessel graft 152, with distal suture structure 160 being substantially aligned with the distal end (not labeled) of blood vessel graft 152 and proximal suture structure 162 being substantially aligned with the proximal end (not labeled) of blood vessel graft 152. In general, this aspect of the disclosed technique enables delivery catheter 156 to accommodate a plurality of lengths of blood vessel graft 152.

In one embodiment of the disclosed technique, blood vessel graft 152 is an artificial vessel graft. In another embodiment, suturing device 154 may be manufactured having a plurality of different lengths to match a plurality of different lengths of artificial vessel grafts. In a further embodiment, if a biosynthetic vascular graft is used, then blood vessel graft 152 is first placed over suturing device 154 and then a patient's (not shown) endothelial cells are grown in blood vessel graft 152.

Reference is now made to FIG. 3A, which is a schematic illustration of additional apparatuses to be used with the heart bypass surgery suturing apparatus of FIG. 2A, generally referenced 200, constructed and operative in accordance with another embodiment of the disclosed technique. Additional apparatuses 200 include a sheath 202 and a double balloon catheter 204. Sheath 202 can be made from silicone, polyurethane (herein abbreviated PU), polyethylene (herein abbreviated PE), polyvinylchloride (herein abbreviated PVC), Dacron® (also known as polyethylene terephthalate or PET), nylon or any other similar material. Sheath 202 is substantially the same length as a blood vessel graft (not shown) used with the disclosed technique. Sheath 202 may optionally include a plurality of radiopaque markers (not shown). The diameter of sheath 202 is slightly smaller than the diameter of the blood vessel graft such that the blood vessel graft can be placed over sheath 202. At the same time, the diameter of sheath 202 is slightly larger than the diameter of a suturing device (not shown) of the disclosed technique, such that the suturing device can be placed within sheath 202. This is shown in greater detail in FIG. 3B. As explained further below, sheath 202 can be used to open the suture structures of the disclosed technique in the case that it is self-expanding.

Double balloon catheter 204 includes a distal balloon 206 and a proximal balloon 208. Each one of distal balloon 206 and proximal balloon 208 may include a plurality of radiopaque markers (not shown). The radiopaque markers can be used as a navigational aid when double balloon catheter 204 is used inside a patient. In addition, as shown below in FIG. 3B, double balloon catheter 204 can be used together with sheath 202. In such an embodiment, radiopaque markers on both sheath 202 and double balloon catheter 204 can be used to determine the position of sheath 202 with reference to double balloon catheter 204. This is useful as a navigational aid when sheath 202 is pulled back, as described below. Distal balloon 206 includes a tube 212 which can be used for inflating and deflating distal balloon 206. Tube 212 is hollow such that a guidewire (not shown) can be passed through an opening 210 in tube 212. Proximal balloon 208 includes a tube 214 which can be used for inflating and deflating proximal balloon 208. Tube 214 is also hollow. As shown in FIG. 3A, the diameter of tube 214 is larger than the diameter of tube 212 such that tube 212 can be passed through tube 214. In this respect, distal balloon 206 and proximal balloon 208 can be inflated and deflated independently of one another. Tube 214 is substantially the same length as tube 212, although only a portion of tube 214 is fully shown in FIG. 3A in order to show how tube 212 is passed through tube 214. The other portion of tube 214 not fully shown is demarcated in FIG. 3A by dotted lines. In a deflated state, distal balloon 206 and proximal balloon 208 have a diameter small enough such that a distal suture structure (not shown) and a proximal suture structure (not shown) can be respectively placed over the distal suture structure and the proximal suture structure. Tubes 212 and 214 are substantially similar to delivery catheter 156 (FIGS. 2A and 2B).

Reference is now made to FIG. 3B, which is a schematic illustration of the additional apparatuses of FIG. 3A being used with the heart bypass surgery suturing apparatus of FIG. 2A, generally referenced 240, constructed and operative in accordance with a further embodiment of the disclosed technique. FIG. 3B shows three possible embodiments of using the additional apparatuses of FIG. 3A with the heart bypass surgery suturing apparatus of FIG. 2A. It is noted that in the embodiments shown in FIG. 3B, guidewires are not shown as part of the heart bypass surgery suturing apparatus. This is only for purposes of clarity. In one embodiment, generally referenced 242, a sheath 202 is used to hold and position a distal suture structure 248 and a proximal suture structure 250. In this embodiment, distal suture structure 248 and proximal suture structure 250 may be made from a self-expanding metal, such as Nitinol, stainless steel or titanium. Distal suture structure 248 and proximal suture structure 250 may exert an outward radial force such that when they are placed within sheath 202, they are held in place. Distal suture structure 248 is placed at a distal end of sheath 202 and proximal suture structure 250 is placed at a proximal end of sheath 202. A blood vessel graft is then placed over sheath 202. This embodiment also includes a delivery catheter (not shown) placed within the hollow of sheath 202. The delivery catheter can be used to align distal suture structure 248 and proximal suture structure 250 respectively with a distal end and a proximal end of the blood vessel graft. When sheath 202 is pulled back, for example once it is placed inside a patient's body, distal suture structure 248 and proximal suture structure 250 are released with their respective outward radial forces causing them to self-expand. Embodiment 242 is now ready to be used in a percutaneous CABG, or TABG surgery according to the disclosed technique.

In another embodiment, generally referenced 244, a distal balloon 252 is used to hold and position distal suture structure 248 and a proximal balloon 254 is used to hold and position proximal suture structure 250. The suture structures in this embodiment are not necessarily made from a self-expanding metal. In this embodiment, the suture structures can be opened via distal balloon 252 and proximal balloon 254, which when inflated, expand and open the suture structures. Distal balloon 252 is inflated, thereby expanding distal suture structure 248 and holding it in place inside a blood vessel graft (not shown). Proximal balloon 254 is inflated, thereby expanding proximal suture structure 250 and holding it in place inside the blood vessel graft. As mentioned above, distal balloon 252 and proximal balloon 254 are inflated independently of one another by separate tubes (not labeled). A blood vessel graft is then placed over embodiment 244. Distal balloon 252 and proximal balloon 254 may be partially inflated in order to respectively fix and hold distal suture structure 248 and proximal suture structure 250 to the blood vessel graft. Distal balloon 252 and proximal balloon 254 may be further inflated once the blood vessel graft is to be used in a percutaneous CABG or TABG surgery in order to open and couple distal suture structure 248 and proximal suture structure 250 with the blood vessel graft. Embodiment 244 is now ready to be used in a percutaneous CABG or TABG surgery according to the disclosed technique.

In a further embodiment, generally referenced 246, distal balloon 252 is used to hold and position distal suture structure 248 and proximal balloon 254 is used to hold and position proximal suture structure 250. In this embodiment, the suture structures may be made from a self-expanding metal. The suture structures and the balloons are then placed within sheath 202. Sheath 202 substantially prevents the sutures structures from self-expanding. Distal balloon 252 is inflated, thereby holding distal suture structure 248 in place inside a blood vessel graft (not shown). Proximal balloon 254 is inflated, thereby holding proximal suture structure 250 in place inside the blood vessel graft. Sheath 202 is used to self-expand distal suture structure 248 and proximal suture structure 250 by pulling it back as described below. A blood vessel graft is then placed over embodiment 246. Distal balloon 252 and proximal balloon 254 may be partially inflated in order to respectively fix and hold distal suture structure 248 and proximal suture structure 250 to sheath 202. Embodiment 246 is now ready to be used in a percutaneous CABG or TABG surgery according to the disclosed technique. It is noted that the three possible embodiments of using the additional apparatuses of FIG. 3A with the heart bypass surgery suturing apparatus of FIG. 2A shown in FIG. 3B can be used for holding the described distal and proximal suture structures of the disclosed technique to a blood vessel graft (not shown). The three embodiments of FIG. 3B can also be used for aiding in navigating the described distal and proximal suture structures of the disclosed technique to their targeted positions in a patient (not shown). The three embodiments of FIG. 3B can further be used for expanding, or aiding in the expansion of the described distal and proximal suture structures of the disclosed technique, which couple the blood vessel graft with another vessel, for example a coronary artery, when they expand.

According to another embodiment of the disclosed technique, the distal and proximal suture structures described above in FIG. 3B can be surrounded by a stent. A blood vessel graft (not shown) is then placed around the stent. In this respect, the whole blood vessel graft is substantially covered from the inside. It is noted that sheath 202, mentioned above, may be embodied as a stent.

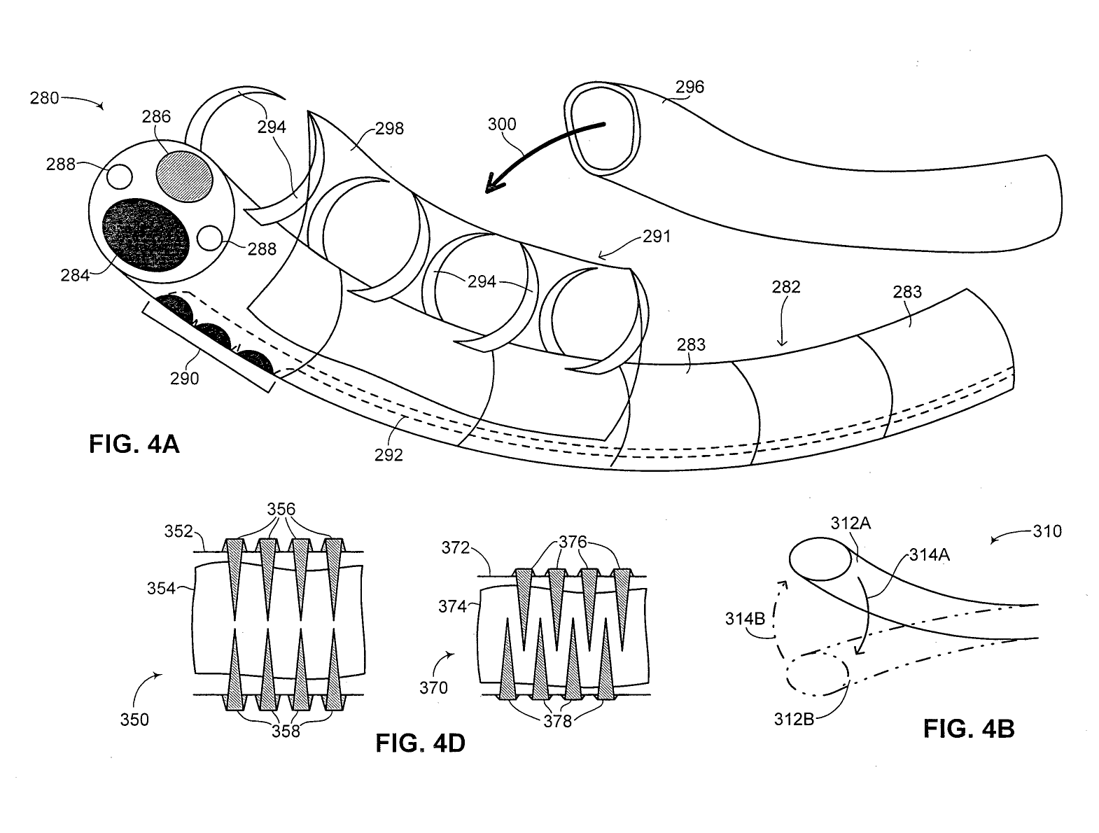

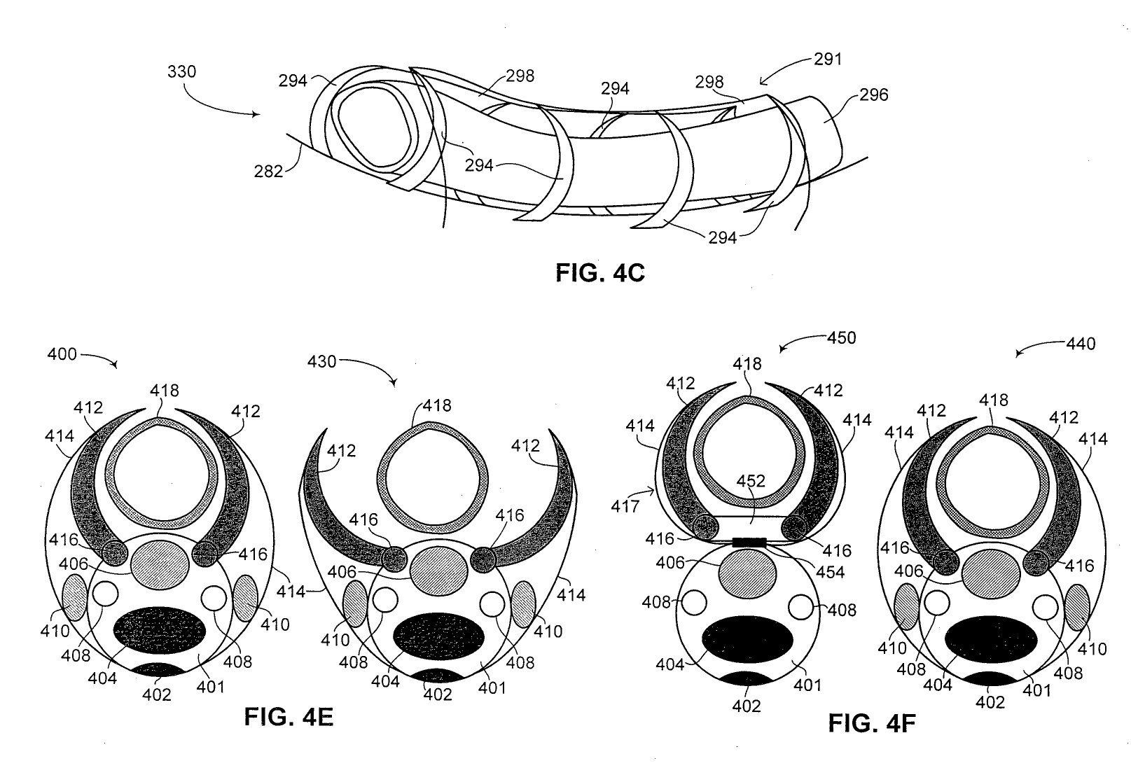

Reference is now made to FIG. 4A, which is a perspective schematic illustration of an endoscopic delivery device, generally referenced 280, constructed and operative in accordance with another embodiment of the disclosed technique. Endoscopic delivery device 280 includes a modified endoscope 282 and a blood vessel graft holder 291. Modified endoscope 282 includes a plurality of flexible backbone sections 283, a working channel 284, a camera 286, a plurality of light sources 288, a plurality of suction holes 290 and a suction tube 292. Blood vessel graft holder 291 includes a plurality of flexible ribs 294 and a shield 298. Blood vessel graft holder 291 also includes a knob (not shown in FIG. 4A) for modifying the position of plurality of flexible ribs 294, as described below. Shield 298 is an optional component in delivery device 280. Shield 298 can be made from Dacron®, plastic, rubber, silicone, PU, PE, PVC, polytetrafluoroethylene (herein abbreviated PTFE), nylon or similar materials. Modified endoscope 282 is similar to prior art endoscopes except that the diameter of modified endoscope 282 is substantially smaller than the diameter of prior art endoscopes. Modified endoscope 282 has a diameter small enough that it can be navigated inside the major blood vessels of a human body while not obstructing the flow of blood within those major blood vessels. In addition, modified endoscope 282 is different than prior art endoscopes in that modified endoscope 282 includes plurality of suction holes 290 and suction tube 292 which can be used to temporarily affix modified endoscope 282 to a moving organ, such as the heart, via a vacuum. It is noted that endoscopic delivery device 280 can be constructed as a multi-use device or a single use disposable device.

Plurality of flexible backbone sections 283 are coupled with one another and enable modified endoscope 282 to be navigated inside a human body, in particular inside the major blood vessels of the human body or in cavities of the human body. Each one of flexible backbone sections 283 is a moving part. Plurality of flexible backbone sections 283 can be controlled and manipulated by a novel handle (not shown in FIG. 4A) for guiding and navigating modified endoscope 282 inside a patient's body. Working channel 284 extends along the length of plurality of flexible backbone sections 283. Working channel 284 enables the injection of contrast agents, the injection of saline solution for cleansing organs, blood vessels, passageways and other bodily elements as well as the injection of CO₂ for inflating various cavities and lumens in the human body if necessary during the TABG surgery of the disclosed technique. Working channel 284 can be also used for suction and for the draining of liquids. Camera 286 is located substantially above working channel 284 and enables a medical practitioner to view what the tip of modified endoscope 282 “sees” especially when modified endoscope 282 is placed inside cavities or lumens of the human body, which are free of blood and enable an image to be captured by camera 286. Camera 286 may be coupled with a plurality of wires (not shown) which run along the length of plurality of flexible backbone sections 283, substantially parallel to working channel 284. This plurality of wires may provide power to camera 286 and may couple camera 286 with a computer (not shown) or a screen (not shown), such as a TV screen, a computer monitor and the like, for receiving video images captured by camera 286. In another embodiment, camera 286 may transmit video images to the computer wirelessly. In a further embodiment, camera 286 may be replaced with a lens (not shown) and an optical fiber (not shown), the optical fiber extending along the length of plurality of flexible backbone sections 283. In such an embodiment, the other end of the optical fiber may be coupled with a viewer (not shown) or a second lens (not shown) for viewing what the tip of modified endoscope 282 “sees.” Camera 286 can also be a combination of a camera and at least one lens or a lens and a light delivery system. The light delivery system may be an optical fiber or a plurality of optical fibers for transferring light to a remote camera (not shown) which is located externally to but coupled with modified endoscope 282. Plurality of light sources 288 are located substantially adjacent to working channel 284 and camera 286 and substantially light the path of modified endoscope 282. In another embodiment of the disclosed technique plurality of light sources 288 is replaced by a single light source (not shown).

Plurality of suction holes 290 is located on the outer surface of modified endoscope 282. Plurality of suction holes 290 is coupled with suction tube 292. Suction tube 292 is coupled with a vacuum device (not shown) for generating suction via plurality of suction holes 290. The suction can be created by first filling suction tube 292 with a liquid, such as saline solution, and then using the vacuum device to suck the liquid thereby creating a vacuum and suction. The suction generated via plurality of suction holes 290 can be used to stabilize and firmly couple endoscopic delivery device 280 to the inner wall of a major blood vessel or to the outer surface of an organ temporarily.

Plurality of flexible ribs 294 is also located on the outer surface of modified endoscope 282, substantially opposite plurality of suction holes 290. Plurality of flexible ribs 294 forms a channel along the length of modified endoscope 282. This channel can be opened and closed longitudinally. In one embodiment, as shown in FIG. 4A, plurality of flexible ribs 294 are coupled with the outer surface of modified endoscope 282. Plurality of flexible ribs 294 can be rotated between an open state and a closed state, shown in more detail below in FIG. 4E. In the open state, a blood vessel graft 296 can be inserted into or released from blood vessel graft holder 291, as shown by an arrow 300. In a closed state, plurality of flexible ribs 294 grasps blood vessel graft 296 adjacent to modified endoscope 282. In the closed state, endoscopic delivery device 280 can be used to transport blood vessel graft 296 to a specified location within a human body. Blood vessel graft 296 may be embedded with the heart bypass surgery suturing apparatus described above in FIG. 2B (not shown in FIG. 4A). On one end, shield 298 couples the tips of plurality of flexible ribs 294 and covers plurality of flexible ribs 294. On the other end, shield 298 may be coupled with a knob (not shown) for opening and closing plurality of flexible ribs 294. Only one side of shield 298 is shown coupling half of plurality of flexible ribs 294 in FIG. 4A. Shield 298 can be manufactured from silicone, PU, PE, PVC, PTFE, nylon or any other similar material. Another side (not shown) of shield 298 couples the other half of plurality of flexible ribs 294. This is shown in greater detail in FIG. 4F below. Shield 298 forms a protective cover for plurality of flexible ribs 294 and substantially prevents plurality of flexible ribs 294 from getting entangled with any part of the cavity or lumen endoscopic delivery device 280 is inserted into. The knob (not shown in FIG. 4A) on blood vessel graft holder 291 can be used to rotate plurality of flexible ribs 294 between its opened state and its closed state. The knob may be a mechanical switch. In another embodiment of the disclosed technique, blood vessel graft holder 291 does not include a knob. In this embodiment, plurality of flexible ribs 294 can be constructed from a shape memory alloy such as Nitinol. In this embodiment, heat (or the lack thereof) can be used to rotate plurality of flexible ribs 294 from a closed state to an open state. In this embodiment, blood vessel graft 296 may be inserted into blood vessel graft holder 291 with plurality of flexible ribs 294 being in a natural open state. Plurality of flexible ribs 294 are then manually closed, or deformed around blood vessel graft 296. When heat is applied to plurality of flexible ribs 294, due to the nature of shape memory alloys, plurality of flexible ribs 294 return to their natural open state, thereby opening up to release blood vessel graft 296. It is noted that shape memory alloys exist which can alternate between two states based on the presence or lack of heat. Plurality of flexible ribs 294 can be made from such shape memory alloys, thereby enabling them to open and close. Heat can be applied to plurality of flexible ribs 294 via an electrical wire (not shown) or via a plurality of electrical wires (not shown). In such an embodiment, each one of plurality of flexible ribs 294 includes a small piece of metal (not shown) such as a lamp filament, which is coupled with the surface of each one of plurality of flexible ribs 294. Each respective small piece of metal is coupled with the electrical wire or with a respective one of the plurality of electrical wires which may run lengthwise (not shown) along modified endoscope 282. Electrical current supplied to the electrical wire heats up the small piece of metal which then transfers heat to plurality of flexible ribs 294. The electrical wire or the plurality of electrical wires can be arranged such that electrical current can be selectively delivered to specified ones of plurality of flexible ribs 294. In this respect, a surgeon can control the opening and closing movements of a single flexible rib, a group of flexible ribs or all of plurality of flexible ribs 294 at once.

Endoscopic delivery device 280 is used to transport blood vessel graft 296 to a desired location in the human body. Prior to insertion in a human body, plurality of flexible ribs 294 are placed in their open state and blood vessel graft 296, embedded with the heart bypass surgery suturing apparatus 150 (FIG. 2A) described above, is placed inside blood vessel graft holder 291. Plurality of flexible ribs 294 is then placed into its closed state, thereby securing blood vessel graft 296 to the outer surface of modified endoscope 282. Endoscopic delivery device 280 is then inserted in a human body (not shown), for example via a major blood vessel, as described further below. Plurality of light sources 288 are turned on to illuminate the path of endoscopic delivery device 280 inside the human body, while camera 286 is used to guide endoscopic delivery device 280 towards a desired position within the human body. Working channel 284 is used to inject a contrast agent or other chemical agent inside the human body to further aid a medical practitioner in navigating endoscopic delivery device 280 to its desired location. Endoscopic delivery device 280, the heart bypass surgery suturing apparatus placed inside blood vessel graft 296 or both may be fitted with radiopaque markers (not shown) which are visible via X-rays. Therefore, real-time fluoroscopy, other real-time imaging technologies, or both can be used to locate the position of endoscopic delivery device 280 within the human body. Shield 298 covers plurality of flexible ribs 294 and prevents them from getting stuck or entangled inside the human body as endoscopic delivery device 280 is moved along a path to its desired location.

Once endoscopic delivery device 280 has reached its desired location, for example distal to a lesion located in a coronary artery, for example a lesion located in the LAD coronary artery, the vacuum device (not shown) is turned on, thereby creating suction at plurality of suction holes 290 via suction tube 292. The desired location is substantially external to the coronary artery with the lesion and is in the vicinity of the pericardium of the heart. Once the suction is turned on, endoscopic delivery device 280 is temporarily affixed to the external surface of the coronary artery with the lesion or to a location on the pericardium substantially adjacent to the coronary artery with the lesion. In the case of TABG surgery according to the disclosed technique, endoscopic delivery device 280 can be firmly coupled with the heart, which is moving, or with one of the major blood vessels exiting or entering the heart via plurality of suction holes 290. With endoscopic delivery device 280 coupled to the heart or to a blood vessel coupled with the heart, endoscopic delivery device 280 substantially moves, or beats with the heart, thereby eliminating any discrepancies between the location of endoscopic delivery device 280 and the location of the heart. In prior art CABG surgery, surgical devices used in off-pump procedures need to factor in the position of a beating heart relative to the position of the surgical devices. According to the disclosed technique such a calculation is obviated once the suction is turned on via suction tube 292, as endoscopic delivery device 280 beats with the heart of a patient. Accordingly, an operator using endoscopic delivery device 280, such as a surgeon or medical practitioner will not see any relative movement between endoscopic delivery device 280 and the heart of the patient. Any images collected by camera 286, which is part of endoscopic delivery device 280, will be presented to the surgeon as a relatively still image even though the patient's heart is moving.

Reference is now made to FIG. 4B, which is a schematic illustration of the movement capabilities of the endoscopic delivery device of FIG. 4A, generally referenced 310, constructed and operative in accordance with a further embodiment of the disclosed technique. Endoscopic delivery device 310 is substantially similar to endoscopic delivery device 280 (FIG. 4A) and includes a modified endoscope (not labeled). A portion of endoscopic delivery device 310 is shown in FIG. 4B. The modified endoscope of endoscopic delivery device 310 can move bilaterally between two positions, schematically shown as a first position 312A and a second position 312B. The movement from one position to the other is shown schematically by a set of arrows 314A and 314B. The general bilateral movement of the modified endoscope of the disclosed technique is substantially similar to the movement capabilities of prior art endoscopes. Unlike the prior art though, the modified endoscope of the disclosed technique is not inserted into a human body via lumens which are normally free of liquids, such as the throat or the rectum. Rather, the modified endoscope is inserted via a blood vessel in which images from the endoscope are substantially obstructed due to the presence of blood. The modified endoscope can also be inserted via a working channel of a blocking balloon of the disclosed technique which is inserted into the human body via a blood vessel. At a puncture location, further described below, the modified endoscope exits the blood vessel and enters a cavity, such as the space surrounding the heart within the pericardium, where a clear image can be captured by the camera (not shown) or lens (not shown) included in the modified endoscope. According to another aspect of the disclosed technique, saline solution, CO₂ or both are injected through a working channel of the modified endoscope into the space surrounding the heart, in order to partially inflate the pericardium while enabling a clearer image and thus improved navigation of the modified endoscope within that space. Once the modified endoscope has been navigated to its target position, the saline solution or CO₂ may be sucked from the space surrounding the heart using the same working channel of the modified endoscope. According to a further aspect of the disclosed technique, the modified endoscope may include a balloon (not shown) at its distal tip. The balloon may be inflated for partially separating the pericardium or for separating a lumen in an individual.

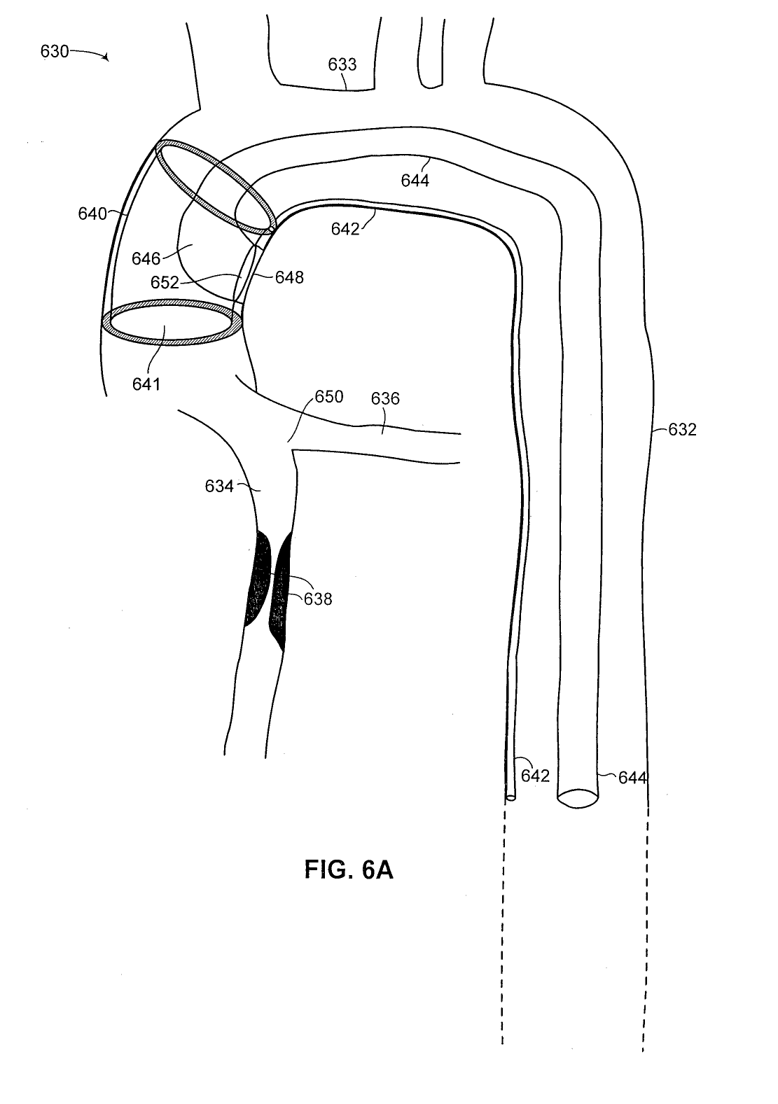

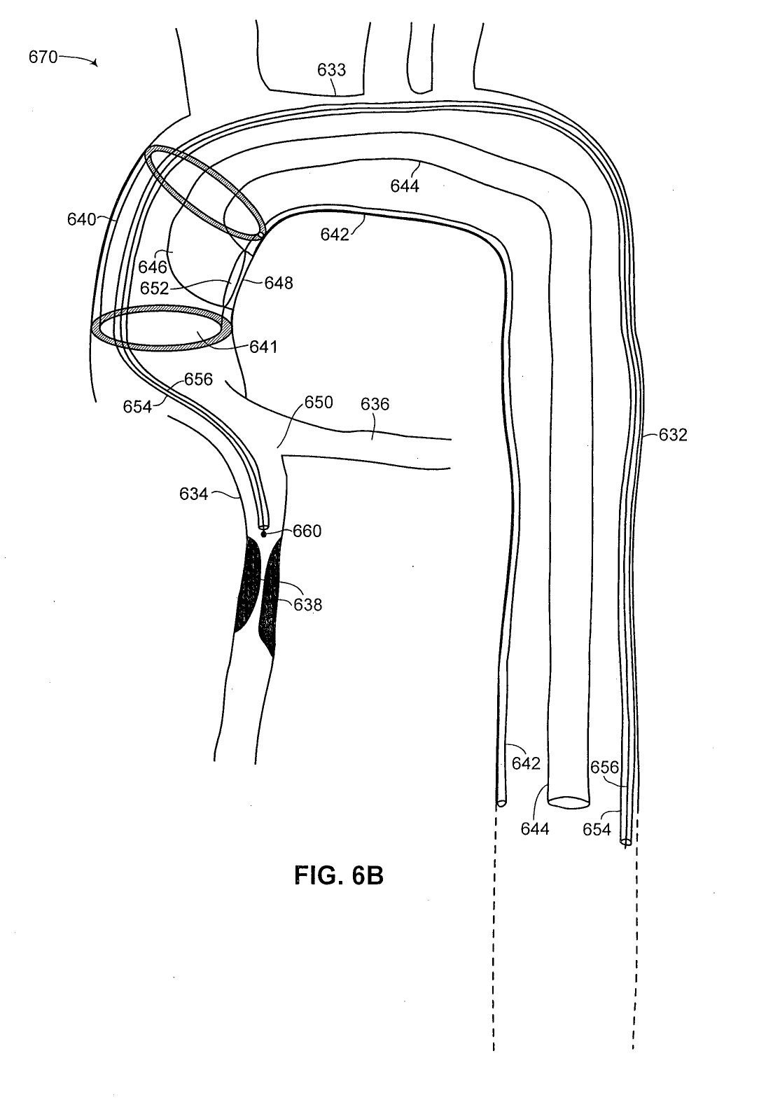

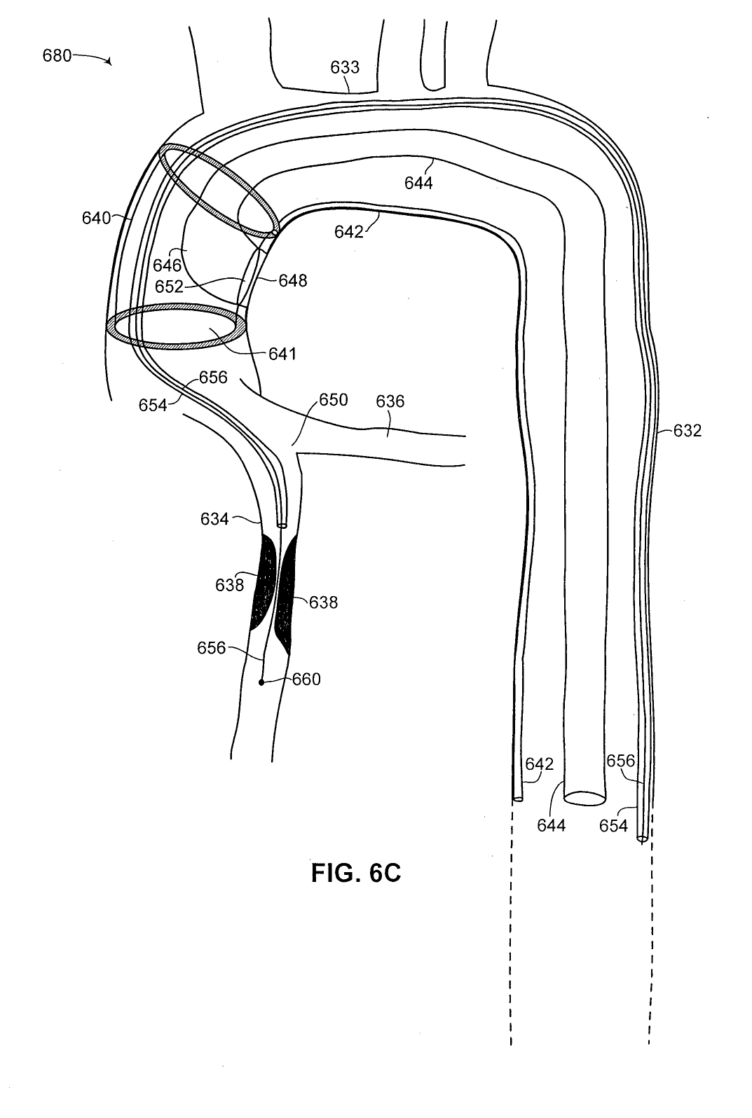

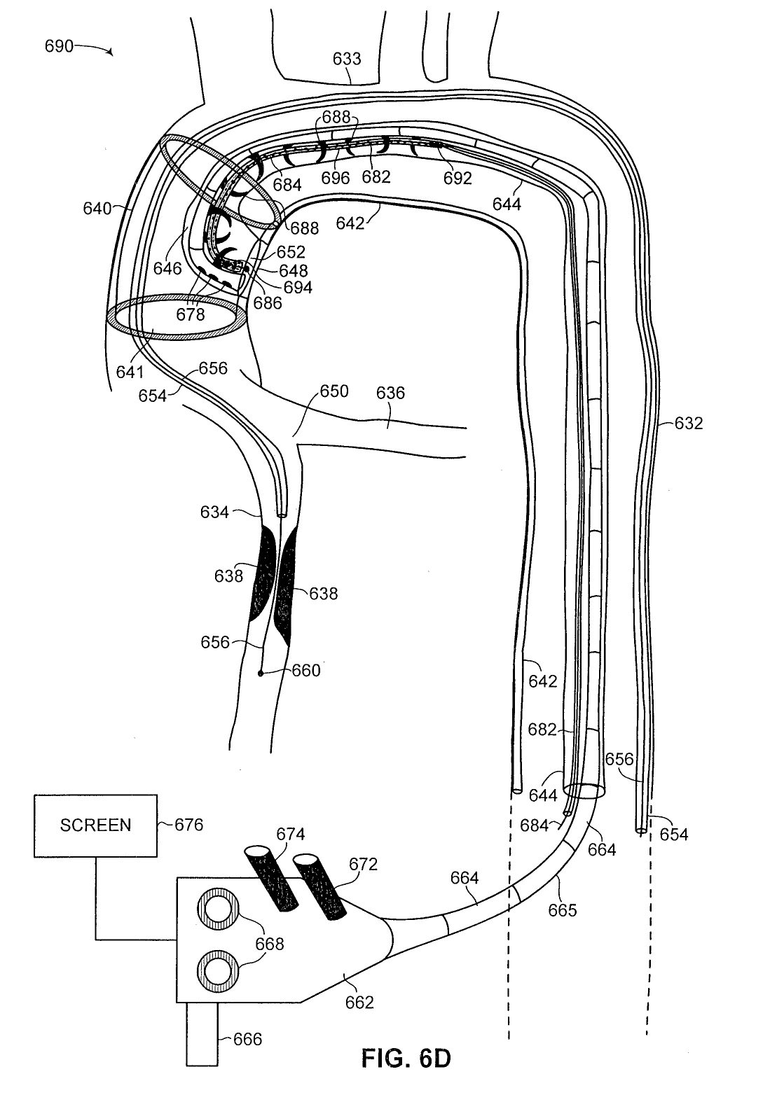

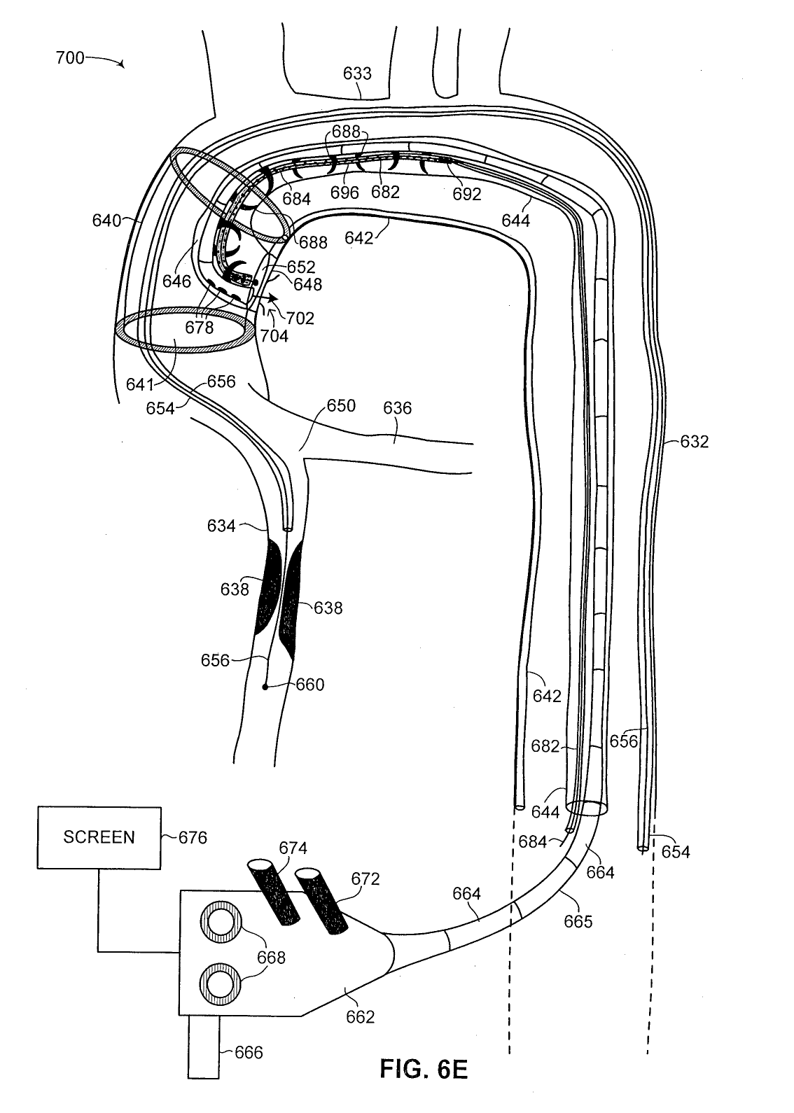

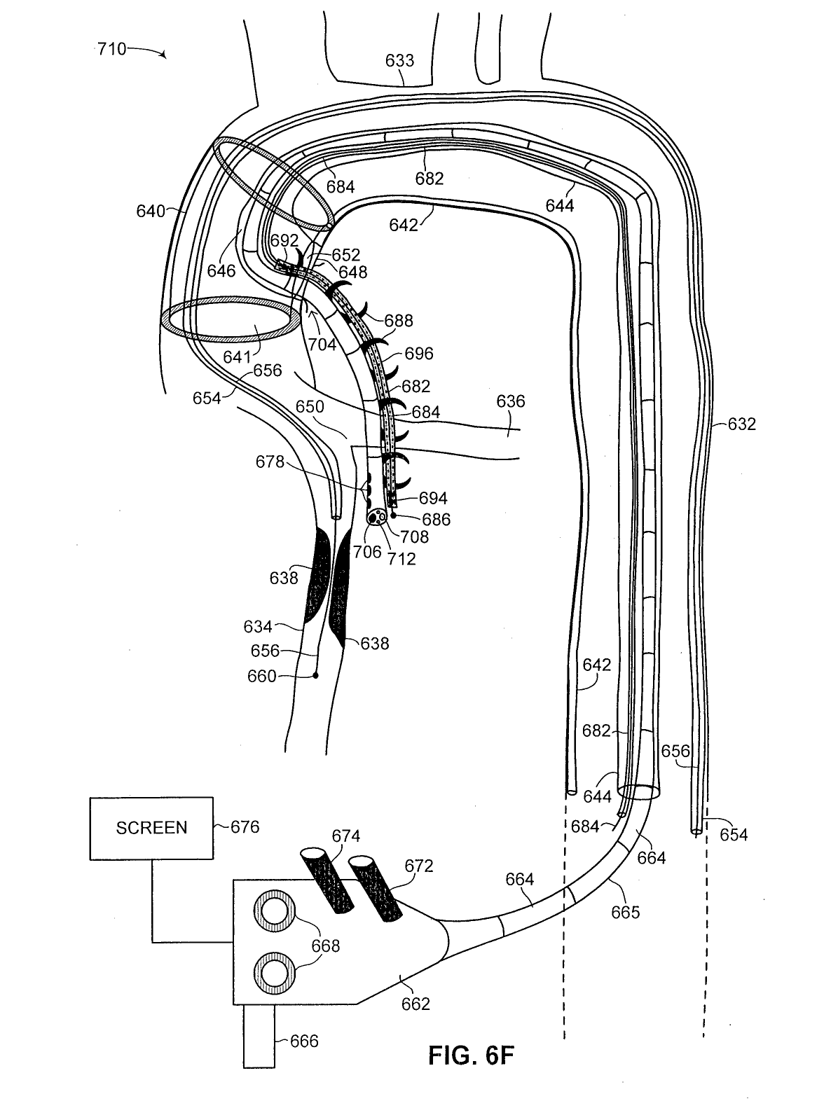

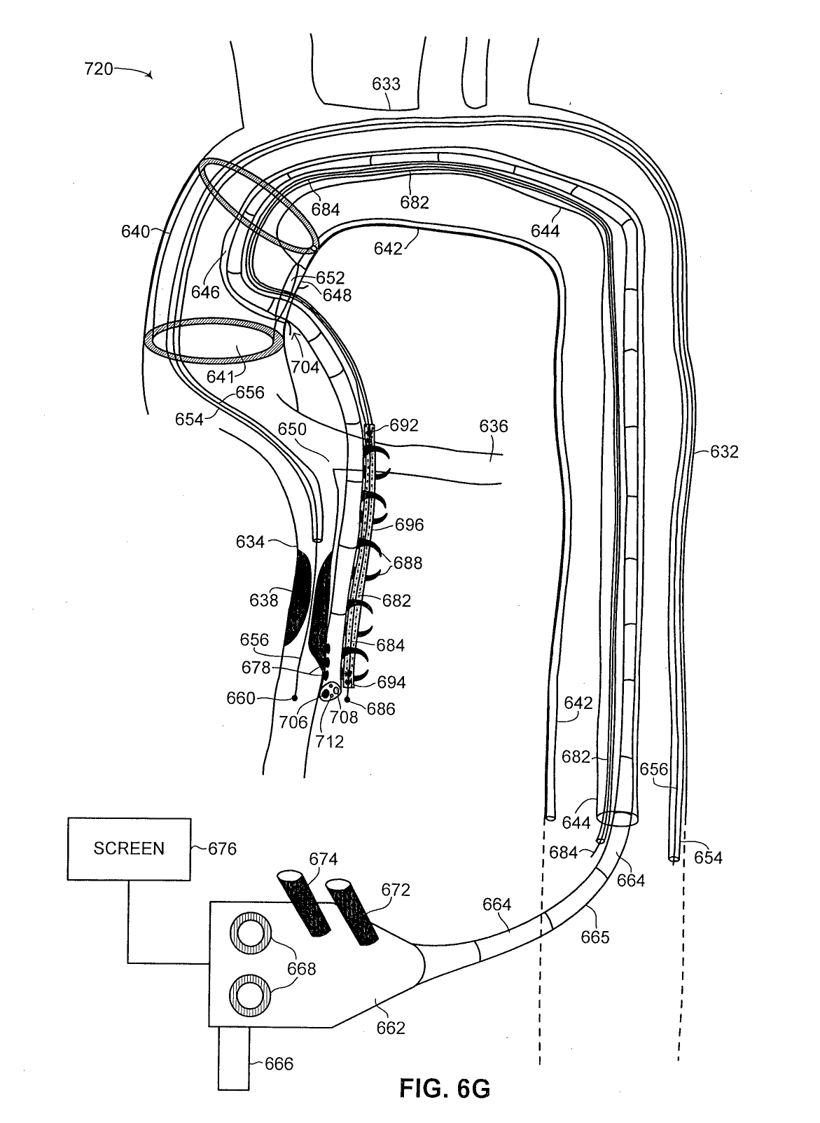

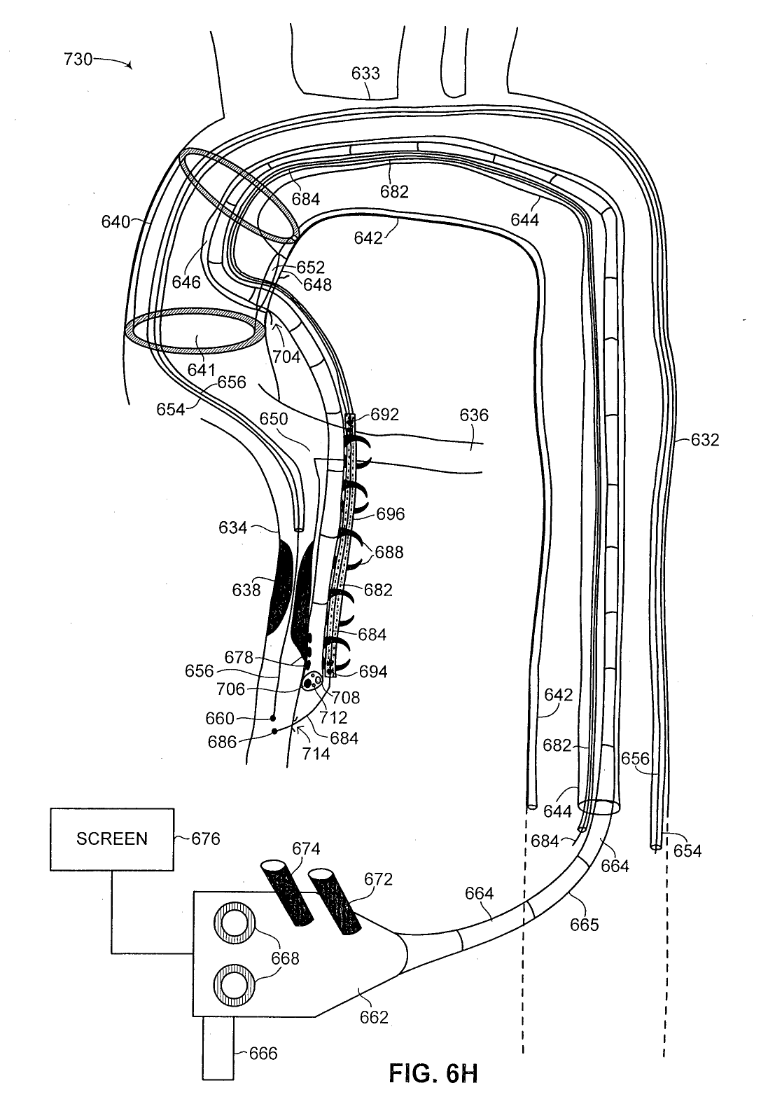

The bilateral movement of endoscopic delivery device 310 enables endoscopic delivery device 310 to be maneuvered through the major blood vessels of the human body, as well as within the cavities and lumens of the body, such as the space surrounding the heart within the pericardium. Initially, endoscopic delivery device 310 is inserted via a working channel (not shown) of a blocking balloon (not shown) of the disclosed technique, further described below in FIG. 6D. Endoscopic delivery device 310 may be inserted over a guidewire (not shown) which guides endoscopic delivery device 310 via the working channel of the blocking balloon to a puncture location (not shown) in the aorta (not shown) of a patient. Once past the puncture location, endoscopic delivery device 310 is navigated via its bilateral movement capabilities in the space surrounding the heart within the pericardium to an attachment location which is distal to a lesion in a coronary artery, further described below in FIG. 6G.

Reference is now made to FIG. 4C, which is another perspective schematic illustration of the endoscopic delivery device of FIG. 4A, generally referenced 330, constructed and operative in accordance with another embodiment of the disclosed technique. Elements in FIG. 4C which are equivalent to elements in FIG. 4A are labeled using identical numbering. FIG. 4C shows blood vessel graft 296 placed inside blood vessel graft holder 291. As shown, plurality of flexible ribs 294 is in its closed position and surrounds and encloses blood vessel graft 296. Plurality of flexible ribs 294 is coupled with modified endoscope 282, of which only a portion is visible in FIG. 4C. In FIG. 4C, both sides of shield 298 are visible, which each respectively couple and cover a portion of plurality of flexible ribs 294.

Reference is now made to FIG. 4D, which is a top orthogonal schematic illustration of different rib arrangements of the endoscopic delivery device of FIG. 4A, generally referenced 350 and 370, constructed and operative in accordance with a further embodiment of the disclosed technique. Rib arrangement 350 shows a first arrangement of the plurality of flexible ribs of the endoscopic delivery device of the disclosed technique. Rib arrangement 370 shows a second arrangement of the plurality of flexible ribs of the endoscopic delivery device of the disclosed technique. Rib arrangement 350 shows a portion of a modified endoscope 352 and a blood vessel graft 354, both of which are similar to modified endoscope 282 (FIG. 4A) and blood vessel graft 296 (FIG. 4A). A first plurality of flexible ribs 356 are arranged on one side of modified endoscope 352 and a second plurality of flexible ribs 358 are arranged on another side of modified endoscope 352. In rib arrangement 350, first plurality of flexible ribs 356 and second plurality of flexible ribs 358 are arranged symmetrically such that each one of first plurality of flexible ribs 356 is aligned opposite a respective one of second plurality of flexible ribs 358. Rib arrangement 370 shows a portion of a modified endoscope 372 and a blood vessel graft 374, both of which are similar to modified endoscope 282 (FIG. 4A) and blood vessel graft 296 (FIG. 4A). A first plurality of flexible ribs 376 are arranged on one side of modified endoscope 372 and a second plurality of flexible ribs 378 are arranged on another side of modified endoscope 372. In rib arrangement 370, first plurality of flexible ribs 376 and second plurality of flexible ribs 378 are arranged in a staggered manner relative to one another. Both of rib arrangements 350 and 370 may enable a plurality of different blood vessel graft diameter sizes, although rib arrangement 370 may be able to accommodate a larger variety in diameter of blood vessel grafts as first plurality of flexible ribs 376 and second plurality of flexible ribs 378 have a greater range of motion due to their staggered positions relative to one another.

Reference is now made to FIG. 4E, which is a cross-sectional schematic illustration of the ribs of the endoscopic delivery device of FIG. 4A in an open and closed state, generally referenced 430 and 400 respectively, constructed and operative in accordance with another embodiment of the disclosed technique. Endoscopic delivery device 430 shows a plurality of flexible ribs 412 in an open state whereas endoscopic delivery device 400 shows a plurality of flexible ribs 412 in a closed state. Equivalent elements in endoscopic delivery devices 400 and 430 are labeled using identical numbering. Endoscopic delivery devices 400 and 430 are substantially similar to endoscopic delivery device 280 (FIG. 4A). Endoscopic delivery devices 400 and 430 include a modified endoscope 401, a suction hole 402, a working channel 404, a camera 406, a plurality of light sources 408, a knob 410, a plurality of flexible ribs 412, a shield 414 and a plurality of hinges 416. A blood vessel graft 418 is enclosed by plurality of flexible ribs 412 in endoscopic delivery device 400. Blood vessel graft 418 is positioned in between plurality of flexible ribs 412 in endoscopic delivery device 430. Plurality of hinges 416 enables plurality of flexible ribs 412 to rotate between their open state and their closed state. In the embodiment shown in FIG. 4E, plurality of hinges 416 are coupled with the outer surface of modified endoscope 410. As shown in FIG. 4E, shield 414 is coupled with the tips (not labeled) of plurality of flexible ribs 412 on one end and with the outer surface of modified endoscope 401 on the other end. In one embodiment, shield 414 is a single piece of material extending from one side of the tips of plurality of flexible ribs 412 to the other side of the tips of plurality of flexible ribs 412. In this embodiment, shield 414 resembles a stocking which can be pulled up over or removed from endoscopic delivery device 400. As mentioned above, shield 414 is an optional component. In another embodiment, shield 414 is made from two pieces of material, with each piece extending from a respective side of the tips of plurality of flexible ribs 412 to the outer surface of modified endoscope 401 adjacent to suction hole 402. In either embodiment, shield 414 comes in contact with knob 410.

Knob 410 enables plurality of flexible ribs 412 to rotate between the open state of endoscopic delivery device 430 and the closed state of endoscopic delivery device 400 and vice-versa by rotating plurality of hinges 416. Knob 410 may be a rotary knob in which plurality of flexible ribs 412 can be opened one by one by rotating knob 410 in a clockwise direction. Such a rotation may cause each one of plurality of flexible ribs 412 to open starting from the most distal flexible rib to the most proximal flexible rib based upon the amount of rotation of knob 410. By rotating knob 410 in a counterclockwise direction, each one of plurality of flexible ribs 412 may close starting from the most proximal flexible rib to the most distal flexible rib, again based upon the amount of rotation of knob 410. In this respect, knob 410 enables blood vessel graft 418 to be partially released, for example, by rotating knob 410 enough in a clockwise direction to release the distal end of blood vessel graft 418 but not enough to release its proximal end. According to the disclosed technique, such a partial release can be used when suturing the distal end of blood vessel graft 418 to a coronary artery (not shown) while still surrounding and gripping the proximal end of blood vessel graft 418. The proximal end of blood vessel graft 418 can then be pulled back towards a major blood vessel, such as the aorta (not shown) using endoscopic delivery device 400. In the closed state, blood vessel graft 418 is enclosed in plurality of flexible ribs 412 and can be transported via endoscopic delivery device 400 to a specified location in the body of a patient. In the open state, blood vessel graft 418 can be inserted into or released from plurality of flexible ribs 412. It is noted that in both the open state and the closed state, shield 414 remains in contact with knob 410.

Reference is now made to FIG. 4F, which is a cross-sectional schematic illustration of different embodiments of the blood vessel graft holder of the endoscopic delivery device of FIG. 4A, generally referenced 440 and 450 respectively, constructed and operative in accordance with a further embodiment of the disclosed technique. Endoscopic delivery device 440 is substantially similar to endoscopic delivery device 400 (FIG. 4E) and shows endoscopic delivery device 440 in a closed state. Equivalent elements in endoscopic delivery device 440 and endoscopic delivery device 400 are labeled using identical numbers. Endoscopic delivery device 450 shows another embodiment of the endoscopic delivery device of the disclosed technique in which the modified endoscope and the blood vessel graft holder are manufactured as two separate elements which can be coupled together. Equivalent elements in endoscopic delivery devices 450 and 440 are labeled using identical numbers. Endoscopic delivery device 450 substantially includes two elements, a modified endoscope 401 and a blood vessel graft holder 417. Modified endoscope 401 includes a suction hole 402, a working channel 404, a camera 406, a plurality of light sources 408 and a sliding channel 454. It is noted that modified endoscope 401 in FIG. 4F does not include a knob. Blood vessel graft holder 417 includes a plurality of flexible ribs 412, a shield 414, a plurality of hinges 416 and a slide connector 452. In endoscopic delivery device 450, shield 414 only surrounds blood vessel graft holder 417 as shown in FIG. 4F. Slide connector 452 couples plurality of hinges 416 together. Slide connector 452 can be inserted into sliding channel 454 thereby coupling blood vessel graft holder 417 with modified endoscope 401. Blood vessel graft holder 417 may also include a torque wire (not shown), coupled with each of plurality of flexible ribs 412 and with a substantially small mechanical relay (not shown), for enabling plurality of flexible ribs 412 to rotate between an open state and a closed state. Other mechanisms (not shown), such as miniaturized versions of those used in window shades, can also be used for rotating plurality of flexible ribs 412 between an open state and a closed state. The opening and closing of plurality of flexible ribs 412 can also be controlled by a push-button mechanism (not shown). It is obvious to one skilled in the art that further mechanisms are also possible for rotating plurality of flexible ribs 412 and that such mechanisms are a matter of design choice. As described below in FIG. 6H, endoscopic delivery device 450 can be used in various embodiments of the disclosed technique for transporting blood vessel graft 418 to a desired location in a patient.

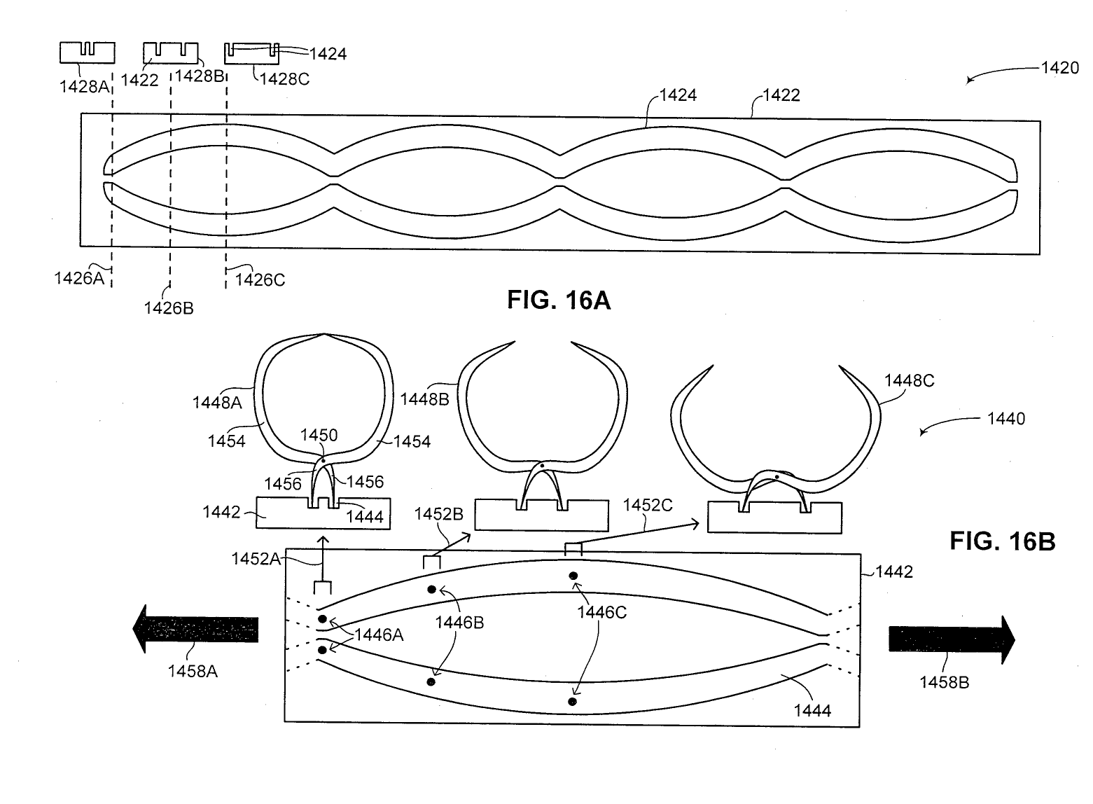

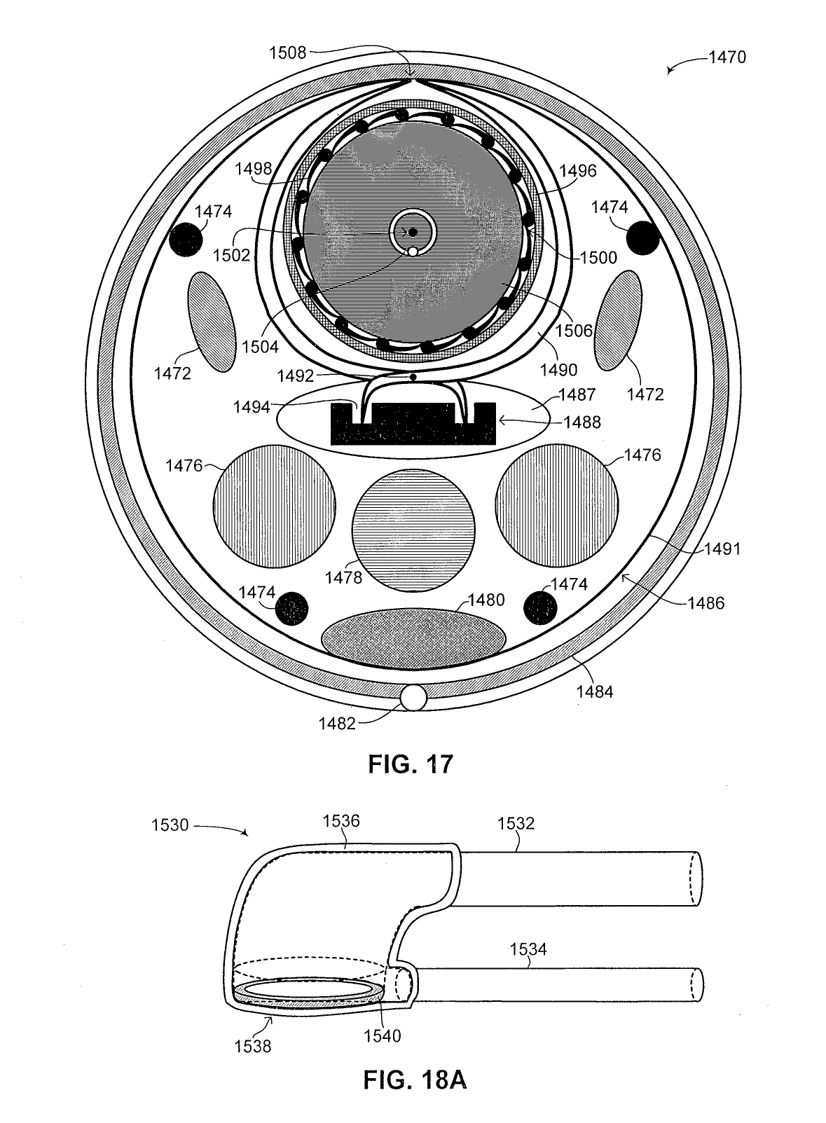

Reference is now made to FIG. 16A which is a top orthogonal schematic illustration of a channel for opening and closing the ribs of an endoscopic delivery device, generally referenced 1420, constructed and operative in accordance with another embodiment of the disclosed technique. Channel 1420 includes a material strip 1422. Material strip 1422 may be made from metal or plastic and is substantially flexible. An upper surface (not labeled) of material strip 1422 is engraved with a track 1424, having a repeating pattern of substantially half-eight shapes. Three dotted lines 1426A, 1426B and 1426C denote cross-sectional slices of channel 1420. Dotted line 1426A shows a cross-section 1428A of channel 1420. Dotted line 1426B shows a cross-section 1428B of channel 1420. Dotted line 1426C shows a cross-section 1428C of channel 1420. As shown in cross-section 1428C, a cross-sectional view of track 1424 shows that track 1424 extends partially through material strip 1422. Along the length of material strip 1422, the distance between the two sides of track 1424 varies based on the repeating substantially half-eight shape of track 1424. As described below in FIG. 16B, channel 1420 can be used to open and close ribs (not shown) which form a part of an endoscopic delivery device (not shown). As described below in FIG. 17, channel 1420 may be included within a section of the endoscopic delivery device and may be moved lengthwise over the length of the endoscopic delivery device for opening and closing the ribs over a blood vessel graft.

Reference is now made to FIG. 16B which is a top and side orthogonal schematic illustration of the channel of FIG. 16A, generally referenced 1440, showing the opening and closing of the ribs of an endoscopic delivery device using the channel, constructed and operative in accordance with a further embodiment of the disclosed technique. Channel 1440 includes a material strip 1442 and a track 1444. For the purposes of clarity only one half-eight shape of track 1444 is shown in FIG. 16B yet it is clear that track 1444 includes a plurality of half-eight shapes (not shown). A portion of each rib is inserted into track 1444 to control the opening and closing of the rib. Three positions of a rib along track 1444 are shown in FIG. 16B, a closed position 1446A, a semi-open position 1446B and an open position 1446C. The black dots in FIG. 16B to which positions 1446A-1446C point to represent the portion of each rib which is inserted into track 1444. A respective side orthogonal view for each of positions 1446A-1446C is shown in FIG. 16B by arrows 1452A-1452C respectively.

In closed position 1446A, a rib 1448A is shown having arms 1454 and legs 1456. Arms 1454 and legs 1456 are coupled together via a hinge 1450, which enables arms 1454 and legs 1456 to rotate around hinge 1450. Legs 1456 are inserted into track 1444. In closed position 1446A, since legs 1456 are relatively close to one another due to the distance of each side of track 1444, arms 1454 remain substantially closed. In semi-open position 1446B, a rib 1448B is shown with its arms (not labeled) and legs (not labeled) spaced further apart due to the increased distance of each side of track 1444. As the distance between the legs of rib 1448B increases, the distance between the arms increases thereby opening up the arms of rib 1448B. In open position 1446C, a rib 1448C is shown with its arms (not labeled) and legs (not labeled) spaced even further apart due to the further increased distance of each side of track 1444. As the distance between the legs of rib 1448C increases, the distance between the arms increases thereby fully opening up the arms of rib 1448C. As described above in FIGS. 4A and 4C, ribs are used to transport a blood vessel graft to the site where the blood vessel graft is to be sutured to a blood vessel which may be occluded. As a rib is moved lengthwise across track 1444 in the direction of either arrow 1458A or 1458B, the distance between the legs of the rib either increases or decreases, thereby opening and closing the arms of the rib. Therefore, ribs may remain closed around a blood vessel graft until the endoscopic delivery device is positioned in the location where the blood vessel graft is to be sutured to a blood vessel. An end (not shown) of material strip 1442 may then be moved to open up the ribs and enable the blood vessel graft to be sutured to the blood vessel. It is noted that each substantially half-eight shape controls the opening and closing of a single rib. Therefore, with a plurality of substantially half-eight shapes along track 1444, a plurality of ribs can be simultaneously opened and closed. It is also noted that hinge 1450 may be embodied as an axis (not shown) extending lengthwise through all the ribs (not shown) which are coupled with the endoscopic delivery device. Therefore, by moving hinge 1450 lengthwise or by moving material strip 1442 lengthwise in the directions of arrows 1458A and 1458B, a rib can be either opened or closed.

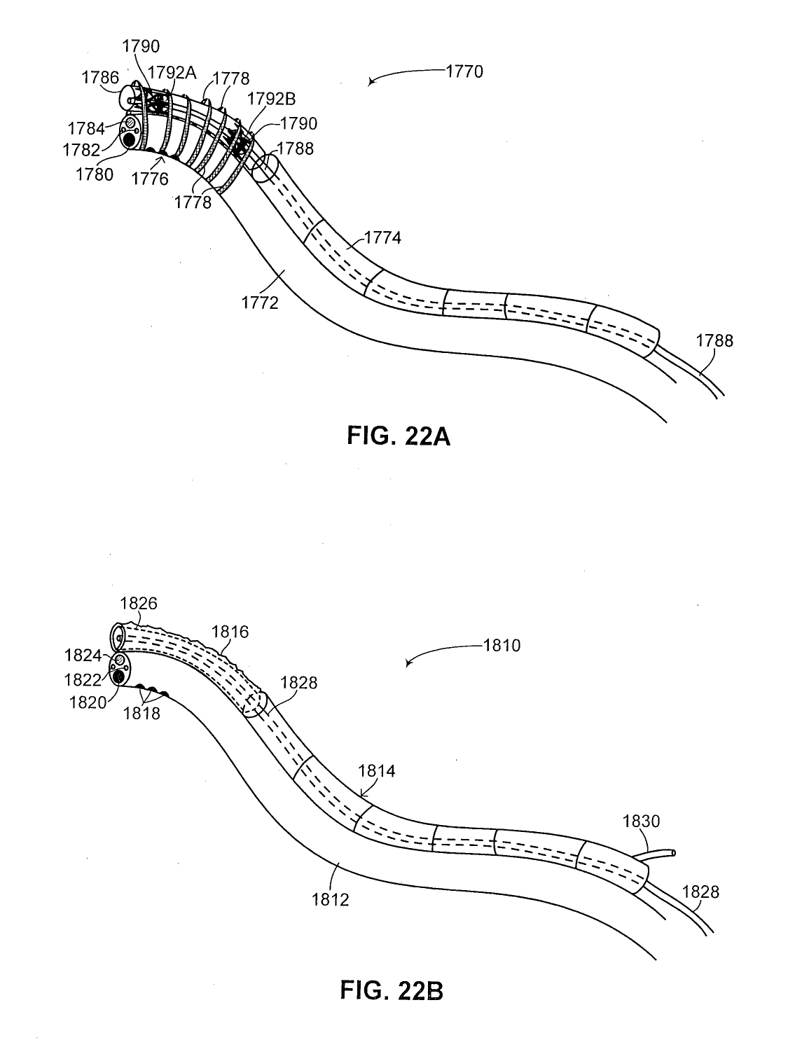

Reference is now made to FIG. 22A which is a schematic illustration of another endoscopic delivery device, generally referenced 1770, constructed and operative in accordance with a further embodiment of the disclosed technique. Endoscopic delivery device 1770 is similar to endoscopic delivery device 280 (FIG. 4A) and includes similar elements. For example, endoscopic delivery device 1770 includes a modified endoscope 1772, which includes a plurality of suction holes 1776, a plurality of flexible ribs 1778, a working channel 1780, a plurality of light sources 1782 and a camera 1784, similar to endoscopic delivery device 280. In addition, endoscopic delivery device 1770 includes a conduit 1774. Conduit 1774 is coupled with modified endoscope 1772 and substantially runs the length of modified endoscope 1772 except for the distal section of modified endoscope 1772 which includes plurality of flexible ribs 1778. Conduit 1774 may be made from plastic, silicon, nylon, rubber and the like. Conduit 1774 is substantially a closed channel. As shown in FIG. 22A, a blood vessel graft 1786 is shown loaded onto endoscopic delivery device 1770, being coupled with endoscopic delivery device 1770 via plurality of flexible ribs 1778. Included within blood vessel graft 1786 are a distal suture structure 1792A and a proximal suture structure 1792B, which are temporarily coupled with blood vessel graft 1786 via a double balloon catheter 1790 which includes a delivery catheter 1788. Plurality of flexible ribs 1778 can open and close via the mechanism (not shown) as described above in FIGS. 16A and 16B.

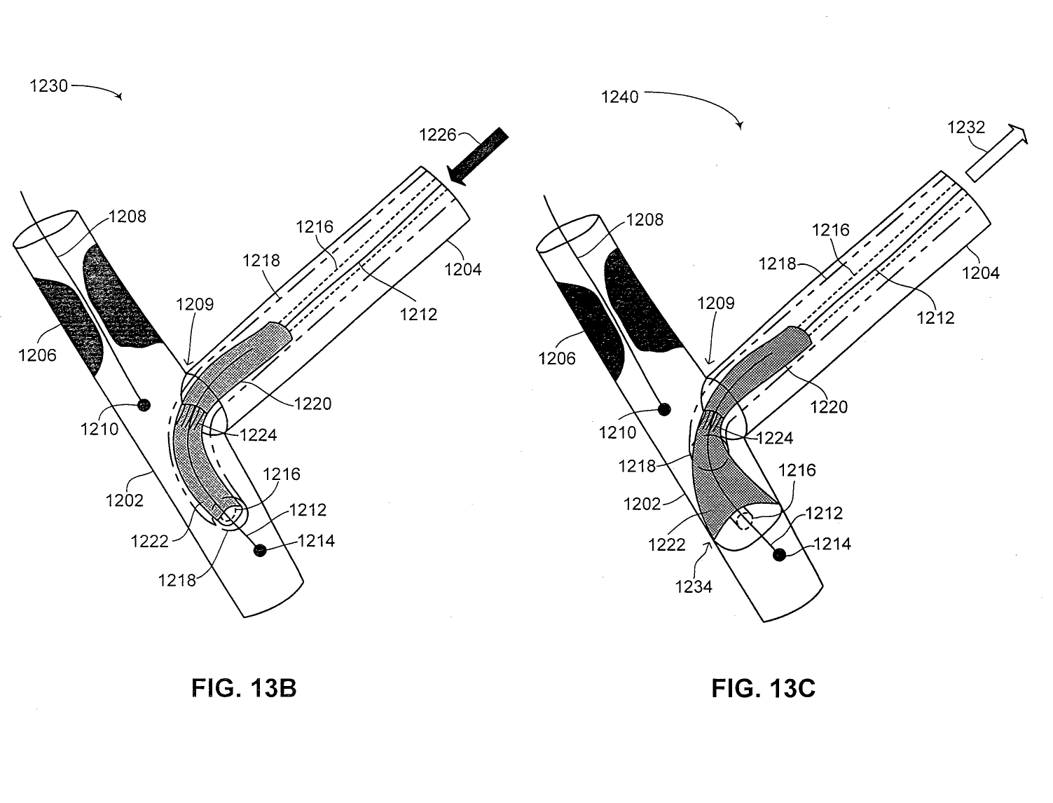

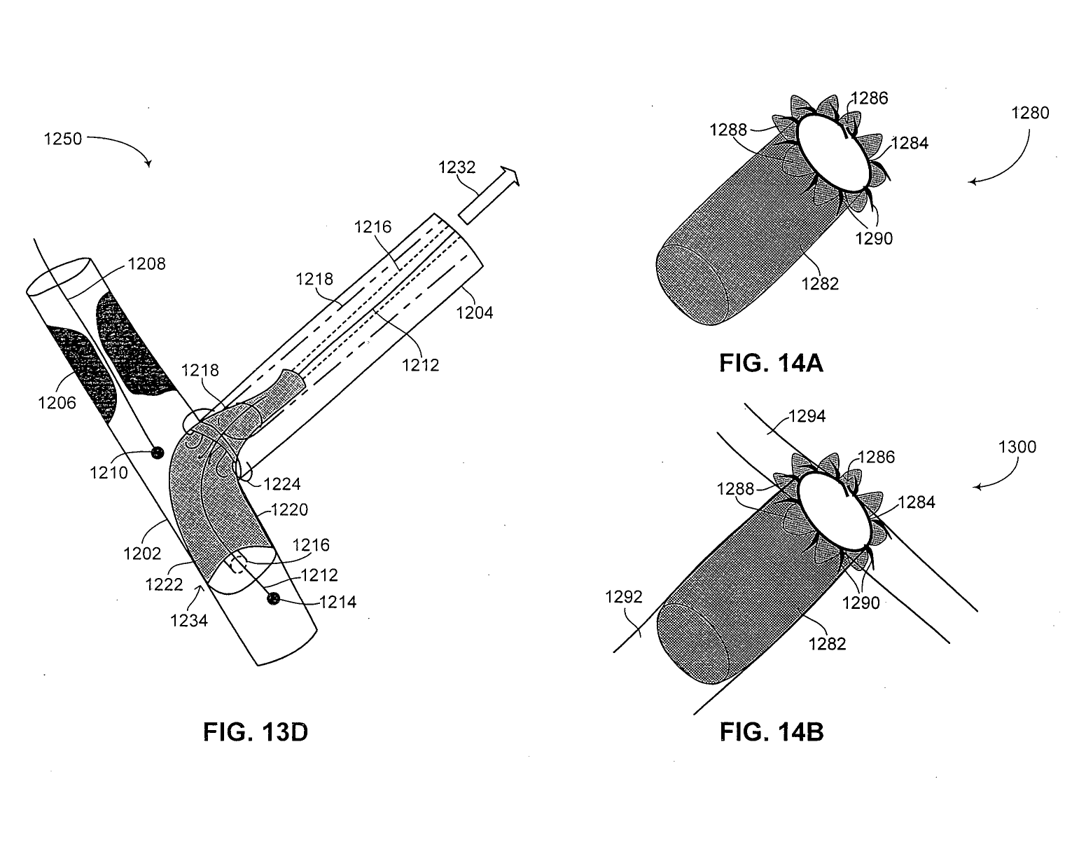

Blood vessel graft 1786 is loaded onto endoscopic delivery device 1770 in the following manner. First, double balloon catheter 1790, distal suture structure 1792A and proximal suture structure 1792B are inserted through conduit 1774 to the distal end (not labeled) of endoscopic delivery device 1770. Second, blood vessel graft 1786 is positioned over double balloon catheter 1790, distal suture structure 1792A and proximal suture structure 1792B at the distal end of endoscopic delivery device 1770. Third, double balloon catheter 1790 is partially inflated, thereby temporarily coupling distal suture structure 1792A and proximal suture structure 1792B to blood vessel graft 1786. Fourth, plurality of flexible ribs 1778 are closed around blood vessel graft 1786. Endoscopic delivery device 1770, loaded with blood vessel graft 1786 is now ready to be used in performing a percutaneous CABG surgery. When at its desired location, blood vessel graft 1786 can be released from endoscopic delivery device 1770 by opening plurality of flexible ribs 1778. As mentioned above, endoscopic delivery device 1770 may be used a plurality of times for implanting a plurality of blood vessel grafts in a patient. In one embodiment, after blood vessel graft 1786 is released from plurality of flexible ribs 1778 and is implanted as described above in FIGS. 13A-13D, for example, endoscopic delivery device 1770 is removed from the patient and reloaded with a second blood vessel graft (not shown). Endoscopic delivery device 1770 is then reinserted into the patient and the second blood vessel graft is implanted. This procedure can be performed multiple times for implanting multiple blood vessel grafts in the patient. According to another embodiment, a plurality of endoscopic delivery devices, such as the endoscopic delivery devices shown in FIGS. 22A and 22B, are preloaded with blood vessel grafts. After a first endoscopic delivery device is inserted into a patient and used to implant a first blood vessel graft, the first endoscopic delivery device is removed from the patient and a second endoscopic delivery device is inserted into the patient and used to implant a second blood vessel graft. This procedure may be repeated using multiple preloaded endoscopic delivery devices for performing a multiple graft TABG surgical procedure.

Reference is now made to FIG. 22B which is a schematic illustration of a further endoscopic delivery device, generally referenced 1810, constructed and operative in accordance with another embodiment of the disclosed technique. Endoscopic delivery device 1810 is substantially similar to endoscopic delivery device 1770 (FIG. 22A) and includes many similar elements. For example, endoscopic delivery device 1810 includes a modified endoscope 1812, which includes a plurality of suction holes 1818, a working channel 1820, a plurality of light sources 1822 and a camera 1824, similar to endoscopic delivery device 1770. In addition, endoscopic delivery device 1810 includes a conduit 1814. Conduit 1814 is coupled with modified endoscope 1812 and substantially runs the length of modified endoscope 1812. Conduit 1814 includes a vacuum tube 1830. A distal end 1816 of conduit 1814 is constructed from a flexible material, such as plastic, silicon, nylon, rubber and the like, which can be inflated and deflated via air pressure using vacuum tube 1830. Distal end 1816 may be embodied as a flexible sheath. Vacuum tube 1830 runs the length of conduit 1814 and is coupled with distal end 1816 (not shown). Using an air compressor (not shown) or a simple injector (not shown) coupled with vacuum tube 1830, distal end 1816 can be inflated and deflated. As shown in FIG. 22B, a blood vessel graft 1826 is shown loaded onto endoscopic delivery device 1810, being coupled with endoscopic delivery device 1810 via distal end 1816. Included within blood vessel graft 1826 is a delivery catheter 1828.

Blood vessel graft 1826 is loaded onto endoscopic delivery device 1810 in the following manner. First, delivery catheter 1828 is inserted through conduit 1814 to distal end 1816 of endoscopic delivery device 1810. Second, blood vessel graft 1826 is positioned over delivery catheter 1828 at distal end 1816 of conduit 1814. Third, distal end 1816 is deflated via a suction generated using vacuum tube 1830, thereby temporarily coupling distal end 1816 to blood vessel graft 1826. Endoscopic delivery device 1810, loaded with blood vessel graft 1826 is now ready to be used in performing a percutaneous CABG surgery. When at its desired location, blood vessel graft 1826 can be released from endoscopic delivery device 1810 by inflating distal end 1816 using vacuum tube 1830. It is noted that distal end 1816 may be inflated by using CO₂, saline solution or both, as an alternative to compressed air.

It is also noted that the endoscopic delivery device of the disclosed technique can be embodied as a combination of the endoscopic delivery devices of FIGS. 22A and 22B. For example, the endoscopic delivery device of the disclosed technique may include a conduit as described above in FIGS. 22A and 22B, a plurality of flexible ribs located at the distal end of the endoscopic delivery device, as described in FIG. 22A, as well as a flexible sheath at the distal end of the endoscopic delivery device as described in FIG. 22B. It is further noted that the endoscopic delivery device of the disclosed technique, in the various embodiments disclosed above, is loaded with a blood vessel graft when the endoscopic delivery device is outside the patient, prior to insertion of the endoscopic delivery device into the patient.

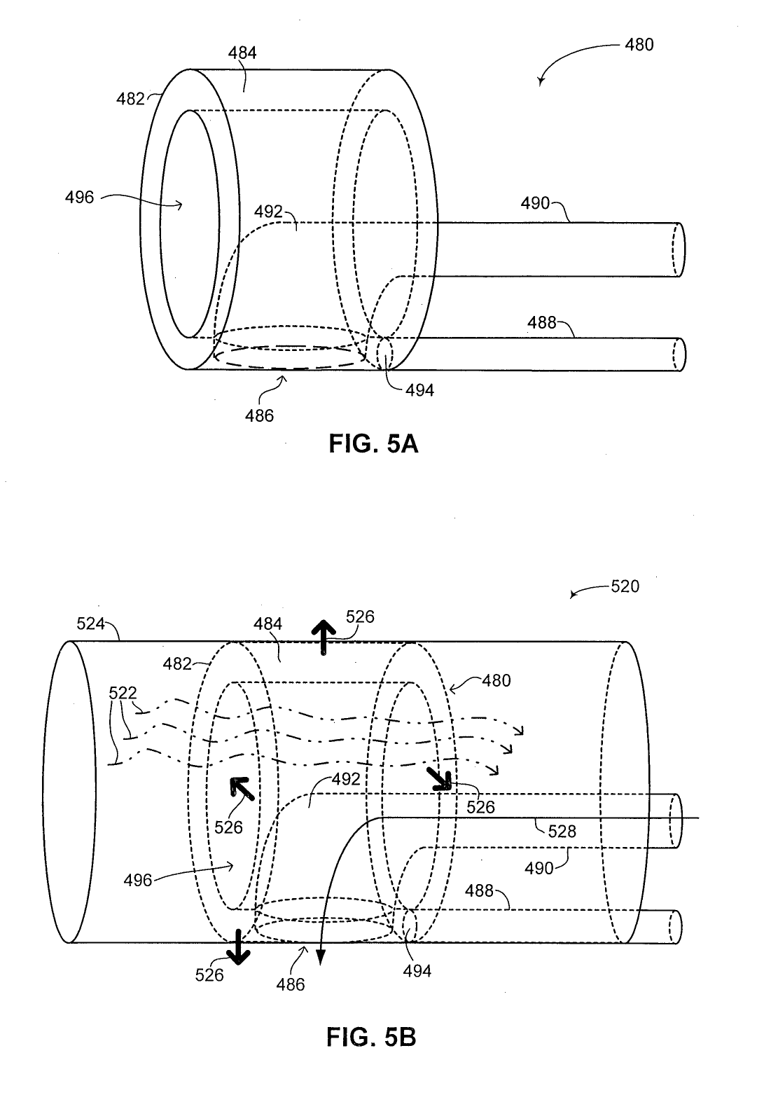

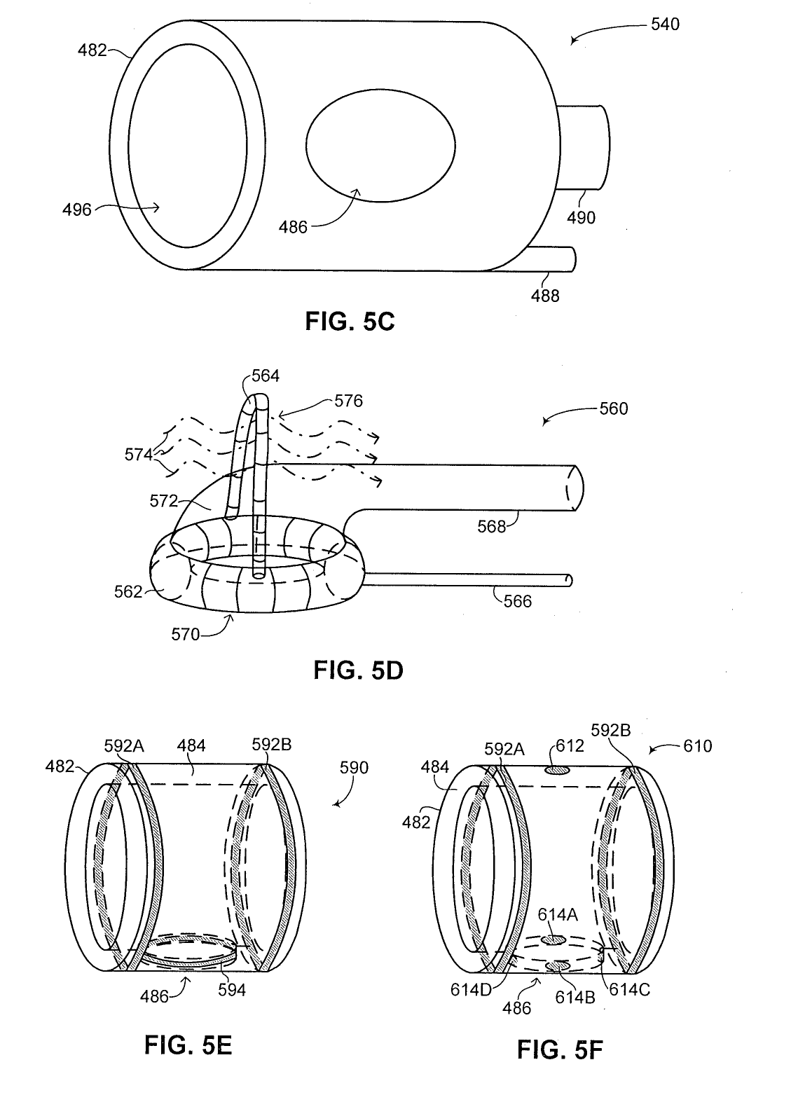

Reference is now made to FIG. 5A, which is a perspective schematic illustration of a blocking balloon, generally referenced 480, constructed and operative in accordance with another embodiment of the disclosed technique. Blocking balloon 480 includes a cylindrical portion 482, a first opening 486, a second opening 496, a first tube 490 and a second tube 488. Blocking balloon 480 also includes a plurality of radiopaque markers (not shown in FIG. 5A) which are described in greater detail below in FIGS. 5E and 5F. First tube 490 is coupled with first opening 486 and second tube 488 is coupled with cylindrical portion 482. Cylindrical portion 482 is a flexible balloon designed in the shape of an open cylinder, as shown by second opening 496, which can be inflated and deflated by injecting, for example saline solution through second tube 488. The portion of first tube 490 which is located within cylindrical portion 482 widens to a form a working space 492. First tube 490 and the related first opening 486 are made of a stiff material such as plastic and are used together as a working channel for the delivery of other devices of the disclosed technique (not shown). Working space 492 is adjacent to first opening 486. First tube 490 represents a working channel by which liquids, other devices or both can be passed through. Second tube 488 is used to inflate and deflate cylindrical portion 482 with a solution such as saline solution. Cylindrical portion 482 itself is hollow as shown by a space 484. Second tube 488 couples with space 484 of cylindrical portion 482 at a coupling point 494. Second tube 488 may be located beneath first tube 490.

Blocking balloon 480 can be inflated and deflated by filling or emptying space 484 via second tube 488. Space 484 can be filled, for example, with saline solution, thereby inflating blocking balloon 480. In general, second tube 488 is substantially smaller than first tube 490. In general cylindrical portion 482 is produced from a flexible material which can be inflated and deflated, such as materials typically used in producing balloon catheters. Such materials include silicone, PU, PE, PVC, Dacron®, nylon and other similar materials. Second tube 488 and first tube 490 however are produced from a more durable material, such as plastic, which is typically used in the production of guiding catheters. It is also noted that in one embodiment of the disclosed technique, first tube 490 and second tube 488 may be coupled together thereby forming a catheter-like device which can be used for inserting into and navigating within a major blood vessel of a patient. In another embodiment of the disclosed technique, blocking balloon 480 may be reshaped for easy navigation within the major blood vessels of the body. In general, the maximum external radius of cylindrical portion 482 when inflated is substantially similar to a typical inner radius of a major blood vessel which blocking balloon 480 may be inserted into, for example the femoral artery, the radial artery and the aorta. Second tube 488 and first tube 490 thereby retain their shape irregardless of whether cylindrical portion 482 is inflated or deflated.

Reference is now made to FIG. 5B, which is a perspective schematic illustration of the blocking balloon of FIG. 5A placed within a blood vessel, generally referenced 520, constructed and operative in accordance with a further embodiment of the disclosed technique. Identical elements in FIGS. 5A and 5B are labeled using identical numbering. As shown in FIG. 5B, blocking balloon 480 is inserted into a blood vessel 524. Blood vessel 524 may be the aorta of a patient. Blood flows through blood vessel 524 in the direction of a plurality of arrows 522. Blocking balloon 480 is initially inserted into the patient in a deflated state via an incision (not shown) into a major blood vessel (not shown), such as the femoral artery or the radial artery. As explained below, using radiopaque markers (not shown) on blocking balloon 480, a surgeon advances blocking balloon 480 to a specified location within the vascular system of the patient, for example, to a specified location on the patient's aorta. Blood (not shown) flows through blood vessel 524 as blocking balloon 480 is navigated to its specified location.

Once blocking balloon 480 is in position, second tube 488 is filled with saline solution (not shown). The saline solution enters cylindrical portion 482 via coupling point 494 and fills space 484. As space 484 fills up with saline solution, cylindrical portion 482 begins to inflate and take shape. As the diameter (not shown) of cylindrical portion 482 approaches the diameter of blood vessel 524, cylindrical portion 482 begins exerting pressure in a radial direction against the inner walls of blood vessel 524, shown in FIG. 5B as plurality of arrows 526. Cylindrical portion 482 is inflated until the pressure exerted by cylindrical portion 482 on blood vessel 524 is sufficient to prevent any of the blood flowing through blood vessel 524 from entering first opening 486. In general, cylindrical portion 482 is designed to inflate to such a pressure. Once no more blood can enter working space 492 via first opening 486 due to the pressure exerted by cylindrical portion 482 on blood vessel 524, working space 492 substantially becomes a blood-free working space in which access to the inner wall of blood vessel 524 is possible via first tube 490 and first opening 486. As mentioned above, first tube 490 may be used to provide saline solution to working space 492 to cleanse working space 492 of any blood that may have entered working space 492 via first opening 486. The saline solution may then be removed by applying suction to first tube 490. According to another embodiment of the disclosed technique, an appropriate sized mandrel (not shown) may be placed in first tube 490, thereby blocking first opening 486 until blocking balloon 480 is fully inflated. The mandrel may then be removed, with working space 492 now being blood-free. Due to the open cylindrical shape of cylindrical portion 482, even when blocking balloon 480 is fully inflated, blood can continue to flow through second opening 496 and thus blood vessel 524, as shown by plurality of arrows 522.

As described further below, first tube 490 enables other devices of the disclosed technique, such as the endoscopic delivery device described above in FIG. 4A-4F and the heart bypass surgery suturing apparatus described above in FIGS. 2A-2B, as well as other tools required to perform the TABG surgery of the disclosed technique, to be inserted there through and to reach the inner wall of blood vessel 524 in a blood-free environment. This is shown by an arrow 528 in FIG. 5B. Also, as described further below, a puncture can be made in the inner wall of blood vessel 524, thereby providing access to the space surrounding the heart within the pericardium. The aforementioned other devices of the disclosed technique can thereby gain access to the space surrounding the pericardium of the heart in a blood-free manner. As such, the TABG surgery of the disclosed technique can be performed off-pump as blood can continue to flow through blood vessel 524 while devices of the disclosed technique are used to suture a bypass graft to a patient's heart in a blood-free environment. It is noted that at this stage of the disclosed technique, when devices, apparatuses and tools are inserted via first opening 486, the disclosed technique switches from being a percutaneous technique to an endoscopic technique.

It is noted that blocking balloon 480 can be used for generating a matter-free environment in other parts of an individual. For example, blocking balloon 480 can be inserted up an individual's anus for generating a fecal-free environment, if access to a lumen or organ is required and most easily accessed via the anus. Blocking balloon 480 can also be inserted into an individual's trachea for generating an air-free environment, if access to a lumen or organ, such as the lungs, is required and most easily accessed via the trachea. Blocking balloon 480 can further be inserted into an individual's esophagus for generating a food-free environment, if access to a lumen or organ, such as the stomach, pancreas or liver, is required and most easily accessed via the esophagus.

Reference is now made to FIG. 5C, which is another perspective schematic illustration of the blocking balloon of FIG. 5A, generally referenced 540, constructed and operative in accordance with another embodiment of the disclosed technique. Whereas FIG. 5A showed blocking balloon 480 from a side perspective view, FIG. 5C shows blocking balloon 540 from a bottom perspective view. Blocking balloons 480 and 540 are equivalent. Identical elements in FIGS. 5A and 5C are labeled using identical numbering. First opening 486 is shown more clearly in FIG. 5C. First opening 486 is coupled with first tube 490. In addition, FIG. 5C shows that second tube 488 and first tube 490 can be coupled with cylindrical portion 482 in a plurality of arrangements, such as side-by-side as shown in FIG. 5C.

Reference is now made to FIG. 5D, which is a perspective schematic illustration of another blocking balloon, generally referenced 560, constructed and operative in accordance with a further embodiment of the disclosed technique. Blocking balloon 560 includes an inner ring portion 562, an arch portion 564, a first opening 570, a second opening 576, a second tube 566 and a first tube 568. Blocking balloon 560 also includes a plurality of radiopaque markers (not shown in FIG. 5D). First tube 568 is coupled with first opening 570 and second tube 566 is coupled with inner ring portion 562. Arch portion 564 is coupled with inner ring portion 562. Inner ring portion 562 and arch portion 564 are both hollow portions. The portion of first tube 568 which is located below arch portion 564 widens to a form a working space 572. Working space 572 is adjacent to first opening 570. First tube 568 represents a working channel by which liquids, other devices or both can be passed through. Second tube 566 may be located beneath first tube 568. Second tube 566 is used to inflate inner ring portion 562 and arch portion 564.

Blocking balloon 560 can be inflated and deflated by filling or emptying the hollow space (not labeled) of inner ring portion 562 and arch portion 564 via second tube 566. As mentioned above regarding FIG. 5A, the hollow space can be filled, for example, with saline solution, thereby inflating blocking balloon 560. Both inner ring portion 562 and arch portion 564 can be filled via second tube 566. In general, second tube 566 is substantially smaller than first tube 568. Inner ring portion 562 and arch portion 564 are produced from a flexible material which can be inflated and deflated. Second tube 566 and first tube 568 are produced from a more durable material, such as plastic, nylon or other similar materials. Second tube 566 and first tube 568 thereby retain their shape irregardless of whether inner ring portion 562 and arch portion 564 are inflated or deflated. When blocking balloon 560 is inserted into a blood vessel (not shown), second opening 576 enables blood to flow freely through blocking balloon 560, shown as a plurality of arrows 574, even when blocking balloon 560 is fully inflated. This was described above in FIGS. 5A and 5B with reference to plurality of arrows 522 (FIG. 5B). As blocking balloon 560 is inflated to a sufficient pressure inside the blood vessel, first opening 570 substantially forms a blood-free environment for working space 572. This was also described above in FIG. 5B. Devices of the disclosed technique, as well as other tools and apparatuses can then be inserted via first tube 568 and given blood-free access to the inner wall (not shown) of the blood vessel.

Reference is now made to FIG. 5E, which is a perspective schematic illustration of a first placement of radiopaque markers on the blocking balloon of FIG. 5A, generally referenced 590, constructed and operative in accordance with another embodiment of the disclosed technique. Identical elements in FIGS. 5A and 5E are labeled using identical numbering. For purposes of clarity, certain elements of the blocking balloon of FIG. 5A have been omitted in FIG. 5E. In FIG. 5E, a plurality of radiopaque markers is placed on cylindrical portion 482 to aid a surgeon in visualizing the position and orientation of the blocking balloon of the disclosed technique while it is advanced in a blood vessel, such as the aorta, of a patient. As shown, the distal and proximal ends of cylindrical portion 482 include a set of radiopaque rings 592A and 592B. First opening 486 also include a radiopaque ring 594. Using X-ray imaging technology, a surgeon can determine the position and orientation of the blocking balloon shown in FIG. 5E as it is advanced inside a patient's vascular system to a specified location.

Reference is now made to FIG. 5F, which is a perspective schematic illustration of a second placement of radiopaque markers on the blocking balloon of FIG. 5A, generally referenced 610, constructed and operative in accordance with a further embodiment of the disclosed technique. Identical elements in FIGS. 5A, 5E and 5F are labeled using identical numbering. For purposes of clarity, certain elements of the blocking balloon of FIG. 5A have been omitted in FIG. 5F. In FIG. 5F, a plurality of radiopaque markers is placed on cylindrical portion 482. As shown, the distal and proximal ends of cylindrical portion 482 include set of radiopaque rings 592A and 592B as in FIG. 5E. Instead of a radiopaque ring, first opening 486 also include a set of radiopaque points 614A, 614B, 614C and 614D. In addition, the section of cylindrical portion 482 opposite first opening 486 also includes a radiopaque point 612. Using X-ray imaging technology, a surgeon can determine the position and orientation of the blocking balloon shown in FIG. 5F as it is advanced inside a patient's vascular system to a specified location. It is obvious to one skilled in the art that other placements or arrangements of the radiopaque markers shown in FIGS. 5E and 5F are possible. It is also noted that the radiopaque marker placements in FIGS. 5E and 5F can be applied to the blocking balloon shown in FIG. 5D with slight modifications as is obvious to one skilled in the art.

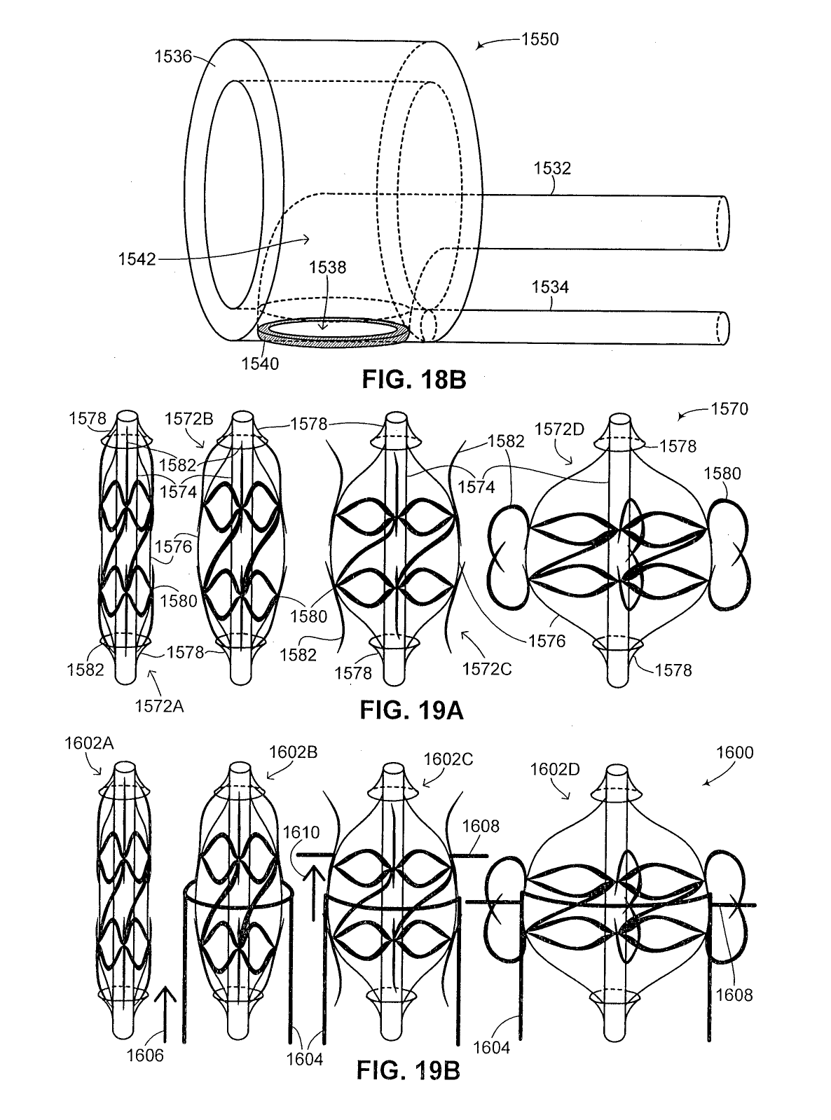

Reference is now made to FIGS. 18A and 18B which are schematic illustrations of a further embodiment of the blocking balloon of the disclosed technique. FIG. 18A is a schematic illustration of a further blocking balloon in a deflated state, generally referenced 1530, constructed and operative in accordance with a further embodiment of the disclosed technique. FIG. 18B is a schematic illustration of the blocking balloon of FIG. 18A an in inflated state, generally referenced 1550, constructed and operative in accordance with another embodiment of the disclosed technique. Equivalent elements in FIGS. 18A and 18B are labeled using equivalent numbering. Blocking balloon 1530 is substantially similar to blocking balloon 480 (FIG. 5A) with a number of noted differences. Blocking balloon 1530 includes a first tube 1532, a second tube 1534, a cylindrical portion 1536, a first opening 1538 and a flexible ring 1540. Cylindrical portion 1536 is shown in FIG. 18A is a deflated state and as such is flush against a portion (not labeled) of first tube 1532. Flexible ring 1540 is positioned at first opening 1538 and is coupled with the tip (not labeled) of first tube 1532. As shown in FIG. 18B, when blocking balloon 1550 is inflated, flexible ring 1540 is positioned around first opening 1538.

When a blocking balloon according to the disclosed technique is positioned in the aortic arch (not shown) of a patient (not shown), the inner wall (not shown) of the aortic arch may not be a smooth surface, for example due to sclerosis or calcification of the aortic arch, even after the blocking balloon is inflated. The inner wall of the aortic arch may be rough and calcified thus preventing a smooth, fluid-tight seal between the inner wall of the aortic arch and the blocking balloon. Flexible ring 1540 is thus coupled with blocking balloon 1550 at the tip of first tube 1532 such that when blocking balloon 1550 is inflated, a tighter seal between the inner wall of the aortic arch and the blocking balloon may be achieved, thus preventing any leakage of blood (not shown) into a working space 1542 of blocking balloon 1550. Flexible ring 1540 can be made from silicon, rubber, plastic or any other soft and adaptable polymer which can adapt to the surface of a tissue when blocking balloon 1550 is inflated.

Reference is now made to FIGS. 6A-6H, which are schematic illustrations of the use of the heart bypass surgery suturing apparatus of FIGS. 2A-3B, the endoscopic delivery device of FIGS. 4A-4F and the blocking balloon of FIGS. 5A-5F in a TABG surgery, generally referenced 630, 670, 680, 690, 700, 710, 720 and 730 respectively, constructed and operative in accordance with another embodiment of the disclosed technique. In general, identical elements in FIGS. 6A-6H are labeled using identical numbering. Due to the many elements shown in some of FIGS. 6A-6H, certain elements in some figures may be shown with a reference number whereas in other figures those same elements may lack the reference number. Each one of FIGS. 6A-6H progressively shows the use of the above mentioned devices of the disclosed technique in performing a TABG surgery according to the disclosed technique.

In general, before a patient undergoes a TABG surgery, the patient undergoes a procedure of medical imaging whereby 2D (two dimensional) images, 3D (three dimensional) images or both are taking of the patient's heart, including the aorta as well as the occluded coronary artery and any lesions in the occluded coronary artery. The medical imaging may be fluoroscopy (for example standard single plane, biplane or rotational angiography fluoroscopy), an ultrasound scan, a CT scan, an MRI scan, a PET scan or other 2D or 3D visualization imaging technology known in the field of medical imaging. The medical imaging is used for pre-planning the TABG surgery and particularly to determine the location on the patient's aorta where a proximal end of a blood vessel graft will be sutured as well as the location on the patient's occluded coronary artery where a distal end of the blood vessel graft will be sutured. These determined locations can be used to determine a three dimensional path, or three dimensional trace, for the blood vessel graft in the patient's body prior to the execution of the TABG surgery of the disclosed technique.

Reference is now made to FIG. 6A, which is a schematic illustration showing a first step in the TABG surgery of the disclosed technique, generally referenced 630, constructed and operative in accordance with a further embodiment of the disclosed technique. FIG. 6A shows the heart of a patient (not shown). For purposes of clarity, only certain portions of the heart of the patient are shown. FIG. 6A shows an aorta 632, an aortic arch 633, a LAD coronary artery 634 and a left circumflex artery 636. LAD coronary artery 634 and left circumflex artery 636 both branch off of the left main coronary artery (not labeled) at an ostium 650 of the left main coronary artery. As shown, LAD coronary artery 634 is occluded by a lesion 638. An incision (not shown) is made in a major blood vessel (not shown), such as the left femoral artery, the right femoral artery, the left radial artery or the right radial artery. A blocking balloon 640, substantially similar to blocking balloon 480 (FIG. 5A), is inserted via the incision and maneuvered into aortic arch 633. An introducer (not shown) is used to create an entry port for blocking balloon 640 via the incision. A guidewire (not shown) may be used to assist with the initial navigation of blocking balloon 640 inside the patient. Blocking balloon 640 includes a first tube 644, a second tube 642, a first opening 652, a second opening 641 and a working space 646. Blocking balloon 640 is inserted and navigated while in a deflated state (not shown). Using radiopaque markers (not shown) on blocking balloon 640 in conjunction with fluoroscopy imaging techniques using either single plane, biplane or rotational angiography real-time fluoroscopy, and based on the determined path of the blood vessel graft, as described above, blocking balloon 640 is maneuvered to a specified location in aortic arch 633 as was determined in the pre-planning procedure described above. It is noted that the navigation and maneuvering of blocking balloon 640 in aortic arch 633 can be performed manually by a medical practitioner or by a robotic manipulator. At this specified location, blocking balloon 640 is inflated by injecting second tube 642 with saline solution. It is noted that first tube 644 and second tube 642 run the length of the path taken by blocking balloon 640 inside the patient's body and specifically within the aorta. In FIGS. 6A-6H, the extension of aorta 632 is shown as dotted lines. It is also noted that first tube 644 and second tube 642 further extend along the aorta until the incision by which they were inserted into the patient's body, even though this is not shown in FIGS. 6A-6H. As blocking balloon 640 is inflated, first opening 652 begins to seal off a portion 648 of the inner wall of aortic arch 633, thereby producing a blood-free space in first tube 644, which is working space 646. Due to the bagel-like shape of blocking balloon 640, including second opening 641, as described above in FIGS. 5A-5F, blood (not shown) continues to flow through aorta 632. Throughout the TABG surgery of the disclosed technique, as described below, working space 646 must remain air-free at all times, either by using saline solution or by using CO₂, in order to avoid collapse of the right side of the heart of the patient. In general, the pressure within first tube 644, working space 646 and the pericardium space (not labeled) of the heart, as shown below in FIGS. 6F-6H once a puncture is made within the inner wall of aortic arch 633, needs to be maintained at a level lower than the pressure of the right side of the heart in order to allow for filling of the right side of the heart.

Reference is now made to FIG. 6B, which is a schematic illustration showing a second step in the TABG surgery of the disclosed technique, generally referenced 670, constructed and operative in accordance with another embodiment of the disclosed technique. Once blocking balloon 640 is in a desired position, a standard guiding catheter 654 is inserted via the incision. Standard guiding catheter 654 is maneuvered through aorta 632, through second opening 641 of blocking balloon 640 into the left main coronary artery of the patient. Standard guiding catheter 654 is maneuvered past ostium 650 into LAD coronary artery 634 to a position which is adjacent yet proximal to lesion 638. It is noted that standard guiding catheter 654 can be navigated and maneuvered through aorta 632 and into LAD coronary artery 634 manually by a medical practitioner or by a robotic manipulator. Ostium 650 may be identified by injecting a contrast agent (not shown) via standard guiding catheter 654, as is done in prior art PCI surgery. A guidewire 656 is then inserted through the lumen (not labeled) of standard guiding catheter 654. Guidewire 656 may be equipped with a position sensor 660 at its tip. Position sensor 660 may be a radiopaque marker. Position sensor 660 may also be an electromagnetic positioning sensor.

It is noted that standard guiding catheter 654 may need to be inserted into the patient via a second incision (not shown). For example, blocking balloon 640 may be inserted into the patient via the left femoral artery (not shown), whereas standard guiding catheter 654 may be inserted into the patient via the right femoral artery (not shown). As mentioned above, other major blood vessels may be used as insertion points for either one of blocking balloon 640 and standard guiding catheter 654. It is also noted that according to the embodiment where standard guiding catheter 654 is first inserted, guidewire 656 is first inserted via the lumen (not shown) of the guiding catheter to a position which is adjacent yet proximal to lesion 638 in LAD coronary artery 634.

Reference is now made to FIG. 6C, which is a schematic illustration showing a third step in the TABG surgery of the disclosed technique, generally referenced 680, constructed and operative in accordance with a further embodiment of the disclosed technique. Once standard guiding catheter 654 is positioned beyond ostium 650, using 3D position tracking equipment (not shown) or fluoroscopy imaging equipment (not shown), a surgeon or operator advances guidewire 656 through lesion 638, based on the location of position sensor 660, to a position in LAD coronary artery 634 which is distal to lesion 638. This distal position may be distal to, adjacent to or at the position on LAD coronary artery 634 where a blood vessel graft is to be sutured distally to lesion 638. Guidewire 656 is then left in this position to be used as a reference. It is noted that in cases where lesion 638 forms a complete occlusion, such as in the case of CTO (chronic total occlusion) where lesion 638 may be calcified and guidewire 656 cannot be navigated past it, it is possible to leave guidewire 656 in a position proximal to lesion 638 (as shown above in FIG. 6B) and to use its position as a reference marker.

It is noted that if the disclosed technique is used for performing a surgical procedure which does not involve the heart, then the steps shown in FIGS. 6B and 6C may be optional. When an anastomosis is performed using the disclosed technique between two vessels or a vessel and a organ in which the organ is not the heart and the vessels are not vessels of the heart, then a guidewire is not necessarily inserted into the patient and maneuvered to a position either adjacent to, proximal to or distal to where the distal end of a vessel graft will be coupled with the vessel or organ.

Reference is now made to FIG. 6D, which is a schematic illustration showing a fourth step in the TABG surgery of the disclosed technique, generally referenced 690, constructed and operative in accordance with another embodiment of the disclosed technique. In fourth step 690, an endoscopic delivery device 662 is inserted via first tube 644 of blocking balloon 640. Endoscopic delivery device 662 is substantially similar to endoscopic delivery device 280 (FIG. 4A). Endoscopic delivery device 662 includes a modified endoscope 665 which includes a plurality of flexible backbone sections 664, a plurality of suction holes 678 and a suction tube (not shown). Modified endoscope 665 also includes a working channel (not shown), a camera (not shown) and a plurality of light sources (not shown), which are shown in FIGS. 6F-6H. As mentioned above, the camera may be replaced by an optical fiber and a lens. Endoscopic delivery device 662 also includes a blood vessel graft holder (not labeled) which includes a plurality of flexible ribs 688, a shield (not shown) and a knob (not shown). The knob is used for opening and closing plurality of flexible ribs 688. Endoscopic delivery device 662 further includes a handle 666, a plurality of control knobs 668, a working channel input 672 and a suction tube input 674. Endoscopic delivery device 662 is a sterile device that can be manufactured as a disposable device or as a reusable device.

Handle 666 provides a surgeon with a grip of endoscopic delivery device 662. The tip of modified endoscope 665 can be manipulated bilaterally, i.e., in two planes. Plurality of control knobs 668 are used for controlling the bilateral movement of the tip of modified endoscope 665. Plurality of control knobs 668 can be replaced by other known elements and devices used for mechanical control of a system. For example, plurality of control knobs 668 may be replaced by a joystick or a robotic manipulator. Endoscopic delivery device 662 is coupled with a screen 676 for displaying the images captured by the camera located on its tip. The plurality of light sources substantially lights the path of the tip of modified endoscope 665 such that images can be captured. Working channel input 672 enables access to the working channel of modified endoscope 665. Working channel input 672 is a sterile input. The working channel of modified endoscope 665 can be used for injecting contrast agents, generating suction and for the introduction of tools via modified endoscope 665. Suction tube input 674 is coupled with plurality of suction holes 678 via the suction tube. Suction tube input 674 is also a sterile input and enables liquids, such as saline solution, to be injected there through. Suction tube input 674 can be coupled with a pump (not shown) or a vacuum device (not shown), for suctioning the injected liquids and thereby creating a suction via plurality of suction holes 678.

Prior to inserting endoscopic delivery device 662 into first tube 644, a blood vessel graft 696 is prepared with a heart bypass surgery suturing apparatus (not labeled). The heart bypass surgery suturing apparatus includes a delivery catheter 682, a guidewire 684, a distal suture structure 694 and a proximal suture structure 692. Guidewire 684 may be equipped with a position sensor 686 on its tip. Position sensor 686 may be a radiopaque marker. Position sensor 686 may also be an electromagnetic positioning sensor. Distal suture structure 694 and proximal suture structure 692 are both explained below in greater detail in FIGS. 7A-7D, 10A-10D and 11A-11B. Guidewire 684 is placed within delivery catheter 682 which is itself placed within blood vessel graft 696. The distal end of blood vessel graft 696 is fitted with distal suture structure 694 whereas the proximal end of blood vessel graft 696 is fitted with proximal suture structure 692. Blood vessel graft 696 is then loaded onto the blood vessel graft holder of endoscopic delivery device 662 by opening plurality of flexible ribs 688, inserting blood vessel graft 696 into the blood vessel graft holder and then closing plurality of flexible ribs 688 around blood vessel graft 696. Modified endoscope 665 with blood vessel graft 696 is then inserted into first tube 644 and maneuvered into working space 646 until the tip (not labeled) of modified endoscope 665 is adjacent to or is within first opening 652. The tip of modified endoscope 665 is thus adjacent to portion 648 of the inner wall of aortic arch 633. As mentioned above, working space 646 is free of blood (not shown) and is isolated from the blood flow in aorta 632. Any blood that entered working space 646 via first opening 652 when blocking balloon 640 was being inflated can be removed using the working channel of modified endoscope 665. For example, saline solution can be injected into working space 646 via the working channel of modified endoscope 665 to cleanse working space 646. The saline solution and any blood or particles in working space 646 can then be removed by coupling working channel input 672 with a pump (not shown) or a vacuum device (not shown), and suctioning working space 646 via the working channel of modified endoscope 665. As mentioned above, endoscopic delivery device 662 can be navigated and maneuvered in first tube 644 and working space 646 manually by a medical practitioner or via a robotic manipulator.

Reference is now made to FIG. 17 which is a front orthogonal schematic illustration of a heart bypass surgery suturing apparatus, generally referenced 1470, including an endoscopic delivery device having the channel of FIGS. 16A-16B, a blocking balloon, a suture structure and a blood vessel graft, constructed and operative in accordance with another embodiment of the disclosed technique. Heart bypass surgery suturing apparatus 1470 includes a blocking balloon 1484, an endoscopic delivery device 1486 and a suture structure 1500. As mentioned below, suture structure 1500 includes a double balloon catheter for delivering the blood vessel graft and a stitching crown suture structure to a desired location in a patient for performing an anastomosis. Blocking balloon 1484 is substantially similar to the blocking balloons described earlier in FIGS. 5A-5F. Endoscopic delivery device 1486 is substantially similar to the endoscopic delivery devices described earlier in FIGS. 4A-4F with a number of differences as described below. Suture structure 1500 is substantially similar to suture structures described earlier in FIGS. 2A-3B.

Blocking balloon 1484 is shown in FIG. 17 in a deflated state and as such is flush against endoscopic delivery device 1486. Blocking balloon 1484 includes an inflating channel 1482, for inflating blocking balloon 1484 as described above. Endoscopic delivery device 1486 includes a plurality of draining channels 1472, a plurality of navigation strings 1474, a plurality of lights 1476, a camera 1478, a suction channel 1480, a longitudinal hollow 1487, a channel 1488, a plurality of ribs 1490 and a sheath 1491. In FIG. 17 only one rib is shown. Plurality of ribs 1490 includes a hinge 1492. Channel 1488 includes a track 1494 into which the legs (not labeled) of plurality of ribs 1490 are inserted into. Plurality of ribs 1490 grasps a blood vessel graft 1496 into which suture structure 1500 is inserted. Suture structure 1500 includes a guidewire 1502, a double balloon catheter 1506, a stitching crown suture structure 1498 and a holding cone 1504.

Navigation strings 1474 are used to maneuver heart bypass surgery suturing apparatus 1470 inside the body of a patient. Holding cone 1504 is described below in FIGS. 19A and 19B in greater detail. Sheath 1491 substantially surrounds the outer surface of endoscopic delivery device 1486 and is coupled with the ends of the arms (not labeled) of plurality of ribs 1490 as shown by an arrow 1508. Sheath 1491 is flexible, as is endoscopic delivery device 1486, such that plurality of ribs 1490 can be opened and closed. In this respect, sheath 1491 applies a slight pressure to plurality of ribs 1490 thereby keeping the legs (not labeled) of plurality of ribs 1490 inside track 1494. In another embodiment, the design of endoscopic delivery device 1486 is such that the legs of plurality of ribs 1490 are kept inside track 1494. Track 1488 is positioned inside longitudinal hollow 1487. Longitudinal hollow 1487 extends over the length of endoscopic delivery device 1486 and is large enough to accommodate track 1488. Track 1488 is substantially the length of longitudinal hollow 1487. Track 1488 can be pulled and pushed lengthwise inside longitudinal hollow 1487, thereby enabling plurality of ribs 1490 to open and close. With plurality of ribs 1490 open, sheath 1491 also opens up thus releasing blood vessel graft 1496.

Reference is now made to FIG. 6E, which is a schematic illustration showing a fifth step in the TABG surgery of the disclosed technique, generally referenced 700, constructed and operative in accordance with a further embodiment of the disclosed technique. In fifth step 700, a puncturing wire 702 is inserted into the working channel of modified endoscope 665 via working channel input 672. Puncturing wire 702 is advanced through the working channel until portion 648. Puncturing wire 702 may also be a puncturing needle (not shown) or a puncturing tool (not shown). Using the camera located at the tip of modified endoscope 665 and watching the displayed images on screen 676 of portion 648, a surgeon creates a puncture 704 in the wall of aortic arch 633. Since puncture 704 is created within first opening 652 which is located within working space 646, puncture 704 is created in an area which is blood-free. Therefore, puncture 704 is isolated from the blood flow in aorta 632.

Reference is now made to FIG. 6F, which is a schematic illustration showing a sixth step in the TABG surgery of the disclosed technique, generally referenced 710, constructed and operative in accordance with another embodiment of the disclosed technique. Once puncture 704 has been created, puncturing wire 702 (not shown in FIG. 6F) is removed from the working channel of modified endoscope 665. In sixth step 710, modified endoscope 665, along with blood vessel graft 696, is advanced through puncture 704 into the space surrounding the heart within the pericardium (not labeled). In FIG. 6F, a working channel 706, a camera 708 and a plurality of light sources 712 of modified endoscope 665 are visible and labeled at the tip of modified endoscope 665. It is noted that the steps of the TABG surgery according to the disclosed technique shown above in FIGS. 6A-6E were substantially steps that were percutaneous in nature, as devices and elements were inserted into aorta 632 via incisions made in the skin of the patient. From sixth step 710 and onwards, as shown in FIGS. 6F-6H and later on in FIGS. 13A-13D, the steps of the TABG surgery according to the disclosed technique are endoscopic in nature, as modified endoscope 665 is used to visualize a part of an organ (e.g., the heart) and to perform procedures (e.g., suturing a blood vessel graft to the heart) on that organ and on cavities of that organ. It is also noted that the steps of the TABG surgery according to the disclosed technique shown above in FIGS. 6A-6E substantially represent percutaneous navigation of tools and devices within a patient's body. From sixth step 710 and onwards, as shown in FIGS. 6F-6H and later on in FIGS. 13A-13D, the steps of the TABG surgery according to the disclosed technique substantially represent endoscopic navigation of tools and devices within a patient's body. This change from percutaneous navigation to endoscopic navigation is unlike the prior art where procedures performed on organs in a patient's body are generally performed either completely percutaneously or completely endoscopically. In the disclosed technique, devices are first navigated through vessels, such as major blood vessels, and then navigated outside those vessels, via a puncture, into a cavity or lumen of an organ or surrounding an organ. It is noted that the endoscopic navigation described in FIGS. 6F-6H can be performed manually via a medical practitioner or via a robotic manipulator.

Navigation of modified endoscope 665 in the space surrounding the heart within the pericardium is achieved using plurality of control knobs 668. Modified endoscope 665 may be fitted with radiopaque markers (not shown). Using fluoroscopic imaging techniques based on the radiopaque markers on modified endoscope 665 and position sensor 660 of guidewire 656, as well as the images captured by camera 708, a surgeon navigates modified endoscope 665 towards lesion 638. In this step, the surgeon navigates modified endoscope 665 based on the path, or trace of blood vessel graft 696 determined above in the pre-planning procedure of medical imaging which was executed before the procedures of the TABG surgery are executed. The path or trace of blood vessel graft 696 inside the patient is thus a pre-planned path or trace. In general, the tip of modified endoscope 665 is to be navigated to a position which is external to the patient's occluded coronary artery, LAD coronary artery 634 in this case, and which is also distal to the lesion in the occluded coronary artery, lesion 638 in this case.

According to another embodiment of the disclosed technique, navigation of modified endoscope 665 can be achieved using electromagnetic tracking sensors (not shown) placed on the tip of guidewire 656 (i.e., if position sensor 660 is an electromagnetic tracking sensor) and on the tip of modified endoscope 665. The relative 3D positions (i.e., relative locations and relative orientations in three dimensions) of the tips of guidewire 656 and modified endoscope 665 can be determined in real-time using electromagnetic tracking devices (not shown) and can be visualized in real-time on screen 676. On screen 676, their respective and relative positions can be shown on a 3D navigational path taken from images of the patient's heart or on a 3D model of the patient's heart based on images of the patient's heart. Such images may be ultrasound images, CT images, MRI images, rotational angiography images or images captured using other known medical imaging devices. In a further embodiment, the endoscopic images captured by camera 708 can be superimposed on the 3D model of the patient's heart. The endoscopic images are thus shown to an operator on screen 676 as real-time footprint images superimposed on the 3D model of the patient's heart. In an additional embodiment of the disclosed technique, navigation of modified endoscope 665 can be achieved by using biplane fluoroscopy images to observe the 3D position of modified endoscope 665 using two imaging views of modified endoscope 665 in real-time and using position sensor 660, embodied as a radiopaque marker, located on guidewire 656 as a reference point. It is also noted in this step that the space surrounding the heart within the pericardium can be inflated using CO₂ to ease the navigation of modified endoscope 665 towards its targeted position on LAD coronary artery 634 distal to lesion 638. In general, CO₂ may be used to inflate the space surrounding the heart within the pericardium because it is easily absorbed by the tissues surrounding the heart. It is further noted that the distal end of modified endoscope 665 may include a balloon (not shown) at its tip. The balloon can be partially inflated to separate the pericardium, thereby easing the navigation of modified endoscope 665 towards its targeted position on LAD coronary artery 634 distal to lesion 638.

Reference is now made to FIG. 6G, which is a schematic illustration showing a seventh step in the TABG surgery of the disclosed technique, generally referenced 720, constructed and operative in accordance with a further embodiment of the disclosed technique. In seventh step 720, lesion 638 is visualized and modified endoscope 665 is navigated to a position adjacent to LAD coronary artery 634. Suction tube input 674 is then injected with saline solution, thereby filling up the suction tube (not shown). Once the suction tube is at least partially full, suction tube input 674 is coupled with a pump (not shown) or a vacuum device (not shown) for suctioning out the saline solution in the suction tube. The act of suctioning out the saline solution creates suction at plurality of suction holes 678. The suction is used to stabilize and temporarily affix the distal end of modified endoscope 665 to LAD coronary artery 634 and to the heart which continues to beat in the off-pump TABG surgery of the disclosed technique. This enables the surgeon to now work on suturing blood vessel graft 696 to LAD coronary artery 634 and aortic arch 633 in a movement-free environment as the suctioned tip of modified endoscope 665 now moves together with heart as the heart beats.

Reference is now made to FIG. 6H, which is a schematic illustration showing an eighth step in the TABG surgery of the disclosed technique, generally referenced 730, constructed and operative in accordance with another embodiment of the disclosed technique. Once modified endoscope 665 is stabilized in relation to LAD coronary artery 634, guidewire 684 is advanced towards LAD coronary artery 634 to a position which is distal of lesion 638. Guidewire 684 is then used to create a puncture 714 in the wall of LAD coronary artery 634, at a position in the occluded coronary artery which is distal to lesion 638. Control of the tip of modified endoscope 665 as well as the visual images captured by camera 708 both assist the user in locating an appropriate puncture location on LAD coronary artery 634 and in creating puncture 714 distally to lesion 638. The position of guidewire 684 as well as the creation of puncture 714 may be imaged internally using camera 708 and externally using real-time fluoroscopy images. The real-time fluoroscopy images are based on the locations of position sensors 660 and 686, embodied as radiopaque markers. The fluoroscopy images may be single plane, biplane or rotational angiography fluoroscopy images. According to another embodiment of the disclosed technique, puncturing wire 702 (not shown in FIG. 6H) is inserted into working channel 706 and is used to create puncture 714 instead of guidewire 684. Puncturing wire 702 is then removed and guidewire 684 is then advanced into LAD coronary artery 634 via puncture 714. In general, it is noted that the 3D locations and orientations of position sensors 660 and 686 can be tracked and determined using electromagnetic tracking devices (not shown). It is also noted that position sensor 660 is used, as shown in FIGS. 6F-6H, as a reference point for determining the relative position of the tip of modified endoscope 665 as well as a finishing marker for aiding a surgeon in guiding the tip of modified endoscope 665 beyond lesion 638 to a position on LAD coronary artery 634 where blood vessel graft 696 is to be sutured to the occluded coronary artery.

It is noted that endoscopic delivery device 662 described in the figures above is substantially similar to endoscopic delivery devices 280 (FIG. 4A) and 440 (FIG. 4F) in which modified endoscope 665 and the blood vessel graft holder are substantially one element. The above described steps, in particular in FIGS. 6D-6H, can also be achieved using an endoscopic delivery device (not shown) which is substantially similar to endoscopic delivery device 450 (FIG. 4F) in which modified endoscope 665 and blood vessel graft holder are separate devices which can be coupled and uncoupled. In this embodiment of the disclosed technique, using such an endoscopic delivery device, in FIGS. 6D-6H, the modified endoscope is inserted into first tube 644 and stabilized distally adjacent to lesion 638 without the blood vessel graft holder coupled with it. Puncturing wire 702 is inserted through working channel 706 to create puncture 714 in LAD coronary artery 634 and guidewire 684 is inserted into first tube 644 by itself and guided into LAD coronary artery 634 via puncture 714. The modified endoscope is then retracted from the patient. The heart bypass surgery suturing apparatus of the disclosed technique is then inserted into blood vessel graft 696 which is then inserted into the blood vessel graft holder. The heart bypass surgery suturing apparatus is then inserted over guidewire 684 and the blood vessel graft holder is then coupled with the modified endoscope. The coupled modified endoscope and blood vessel graft holder are then reinserted into the patient and the TABG surgery of the disclosed technique continues as described below. According to another embodiment of the disclosed technique, again using an endoscopic delivery device which includes two elements, once the modified endoscope is removed from the patient, after puncture 714 was created and guidewire 684 was advanced into LAD coronary artery 634 via puncture 714, the blood vessel graft holder, grasping the heart bypass surgery suturing apparatus and the blood vessel graft, is inserted into the patient over guidewire 684. In this embodiment, the blood vessel graft holder is a standalone device (not shown) which is inserted by itself into first tube 644 without the presence of the modified endoscope. This embodiment may enable better manipulation of the modified endoscope inside the patient, since its cross-sectional area will be smaller without the blood vessel graft holder coupled with it. At the same time, guidewire 684 has a higher chance of becoming dislodged from puncture 714 when the modified endoscope is removed from the patient. According to a further embodiment of the disclosed technique, again using an endoscopic delivery device which includes two elements, the modified endoscope is inserted into first tube 644 and stabilized distally adjacent to lesion 638 without the blood vessel graft holder coupled with it. The blood vessel graft holder, grasping the heart bypass surgery suturing apparatus and the blood vessel graft, is prepared outside the patient and is inserted into the patient over a delivery catheter (not shown) or over guidewire 684 via a conduit (not shown) coupled with the modified endoscope. The conduit is substantially a closed channel. Such a modified endoscope with a conduit is described below in FIGS. 22A and 22B. The blood vessel graft holder is maneuvered inside the conduit until its desired location distally adjacent to lesion 638. The TABG surgery of the disclosed technique then continues as described above in FIGS. 6G and 6H. This embodiment may enable better manipulation of the modified endoscope inside the patient, since its cross-sectional area will be smaller without the blood vessel graft holder initially coupled with it. Guidewire 684 may also have a reduced chance of becoming dislodged from puncture 714 since it is inserted through a closed channel.

According to the disclosed technique, portion 648 of the inner wall of aortic arch 633 may be temporary closed in case the TABG surgery described in FIGS. 6A-6H using the disclosed technique fails. Portion 648 may be temporarily closed using a known patent foramen ovale (herein abbreviated PFO) closure device, such as the AMPLATZER® (by AGA Medical), the HELEX® Septal Occluder (by Gore), the CardioSEAL™ and STARFlex™ devices (by Nitinol Medical Technologies, Inc.) or the Premere™ PFO Closure System (by St. Jude Medical). In such a scenario, blocking balloon 640 is kept inflated, while endoscopic delivery device 662 is removed from first tube 644. The known PFO closure device (not shown) is then inserted through first tube 644 into working space 646 until first opening 652. The known PFO closure device is then inserted into portion 648 to temporarily close portion 648 which is substantially a puncture in the inner wall of aortic arch 633. A patient can then be moved to an operating room where a traditional CABG surgical procedure can be performed and the other components of the disclosed technique, such as standard guiding catheter 654 and blocking balloon 640, can be removed.

As described later on in FIGS. 13A-13D, additional steps are executed in the TABG surgery of the disclosed technique to complete the delivery and the suturing of blood vessel graft 696 to the patient's occluded coronary artery and aortic arch. As described below, at least a portion of plurality of flexible ribs 688 is opened and delivery catheter 682 is used to advance the distal end of blood vessel graft 696 into LAD coronary artery 634 via puncture 714. Delivery catheter 682 is guided into puncture 714 over guidewire 684. Navigation of delivery catheter 682 and blood vessel graft 696 can be achieved using single plane, biplane or rotational angiography fluoroscopy imaging based on the locations of position sensors 660 and 686, embodied as radiopaque markers, as well as by visualizing the area of LAD coronary artery 634 distal to lesion 638 using images captured with camera 708. Distal suture structure 694 is then released, as described below, thereby suturing the distal end of blood vessel graft 696 to puncture 714. The other portion of plurality of flexible ribs 688 is then opened and modified endoscope 665 is then pulled back enough such that its tip is within aortic arch 633. Delivery catheter 682 is then pulled back enough such that the proximal end of blood vessel graft 696 is advanced into aortic arch 633 via puncture 704. Proximal suture structure 692 is then released, as described below, thereby suturing the proximal end of blood vessel graft 696 to puncture 704. Blood vessel graft 696 now represents a bypass route for blooding exiting the heart to reach LAD coronary artery 634. Before the devices of the disclosed technique are removed from the patient, the tip of modified endoscope 665 may be positioned facing the proximal end of blood vessel graft 696. Working channel 706 can then be injected with saline solution or with a contrast agent to verify the bypass route and the flow of liquids via the bypass route created by blood vessel graft 696. Once the bypass route has been verified, standard guiding catheter 654 and guidewire 656 are removed from the patient. Modified endoscope 665, delivery catheter 682 and guidewire 684 are then removed from the patient. Blocking balloon 640 is then deflated by suctioning out the saline solution injected into blocking balloon 640 via second tube 642. As blocking balloon 640 deflates blood begins to flow into LAD coronary artery 634 via blood vessel graft 696. Blocking balloon 640 is then removed from the patient and the patient's incisions are then sewn up. It is noted that all the above steps can be repeated without removing blocking balloon 640. Blocking balloon 640 can be slightly deflated and then navigated to another location in aortic arch 633 where it is inflated again. A second TABG surgery can then be executed using a second blood vessel graft. The TABG procedure of the disclosed technique can thus be performed multiple times while using a single blocking balloon.

Reference is now made to FIGS. 7A-7B, which are schematic illustrations of a suture structure used to suture a blood vessel graft to a blood vessel, generally referenced 750 and 780 respectively, constructed and operative in accordance with a further embodiment of the disclosed technique. Identical elements in FIGS. 7A and 7B are labeled using identical numbering. FIG. 7A shows two views of the suture structure. A view 766 shows the suture structure placed in a blood vessel graft before it has been released thereby coupling the blood vessel graft with a blood vessel. A view 768 shows a perspective representation of the suture structure in a closed state, as described below. With reference to view 766, a suture structure 756 is placed within a blood vessel graft 754 at the distal end of blood vessel graft 754. Suture structure 756 includes a plurality of distal thorns 758, a plurality of proximal thorns 760 and a connector 762. Plurality of distal thorns 758 slightly protrude from the distal end of blood vessel graft 754. The tips of plurality of distal thorns 758 and plurality of proximal thorns 760 are sharp. The protruding portion of plurality of distal thorns 758 can be used to pierce a blood vessel, such as LAD coronary artery 752, shown by an arrow 764. LAD coronary artery 752 is brought merely as an example of a blood vessel which can be sutured with blood vessel graft 754 using suture structure 756. In an embodiment of the disclosed technique using suture structure 756, a puncturing wire (not shown) may not be needed to create an initial puncture (not shown) in the wall of an aortic arch (not shown) nor to create a puncture (not shown) in LAD coronary artery 752, as the protruding portion of plurality of distal thorns 758 can create both such punctures.

Suture structure 756 can be used as both the distal suture structure (as shown in FIGS. 7A and 7B) as well as the proximal suture structure (not shown) of the disclosed technique. Suture structure 756 forms part of the heart bypass surgery suturing apparatus described above in FIGS. 2A and 2B. For reasons of clarity, some elements of the heart bypass surgery suturing apparatus described above, such as a guidewire, a delivery catheter, a sheath and a double balloon catheter, are not shown in FIGS. 7A and 7B. If suture structure 756 is used as the proximal suture structure of the disclosed technique, then suture structure 756 is mounted in a similar manner at a proximal end (not shown) of blood vessel graft 754, with the plurality of distal thorns protruding slightly from the proximal end of blood vessel graft 754. Suture structure 756 can also be used in only the distal end of blood vessel graft 754 to suture blood vessel graft 754 to a blood vessel or in the distal end of an existing artery, such as the LIMA (not shown), which may be rerouted and reattached to the heart at another location. It is also noted that suture structure 756 may be built into blood vessel graft 754 if blood vessel graft 754 is an artificial graft or a semi-artificial graft.

With reference to view 768, suture structure 756 is constructed from metal. Examples of such metals can include Nitinol, titanium, stainless steel or other metals used in medical procedures which can be left in the body without fear of infection or contamination. Connector 762 is substantially circular in shape and is coupled with plurality of distal thorns 758 and plurality of proximal thorns 760. Connector 762 may be a ring or a plurality of rings. Connector 762 may also be a flexible ring, as shown below in FIG. 7C. Respective ones of plurality of distal thorns 758 and plurality of proximal thorns 760 may be constructed as a single piece of metal. The coupling of connector 762 with both plurality of distal thorns 758 and plurality of proximal thorns 760 gives suture structure 756 a crown-like shape. In general, suture structure 756 has two states, a closed state and an open state. The open state can also be referred to as a deployed state or a released state.

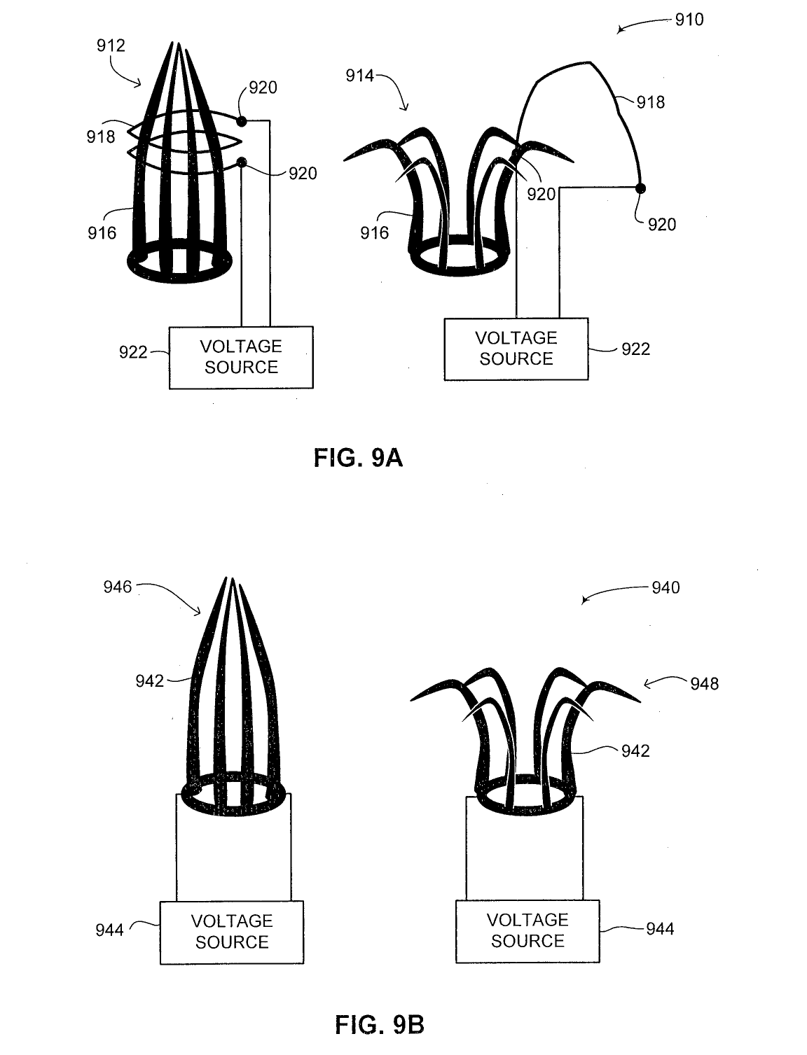

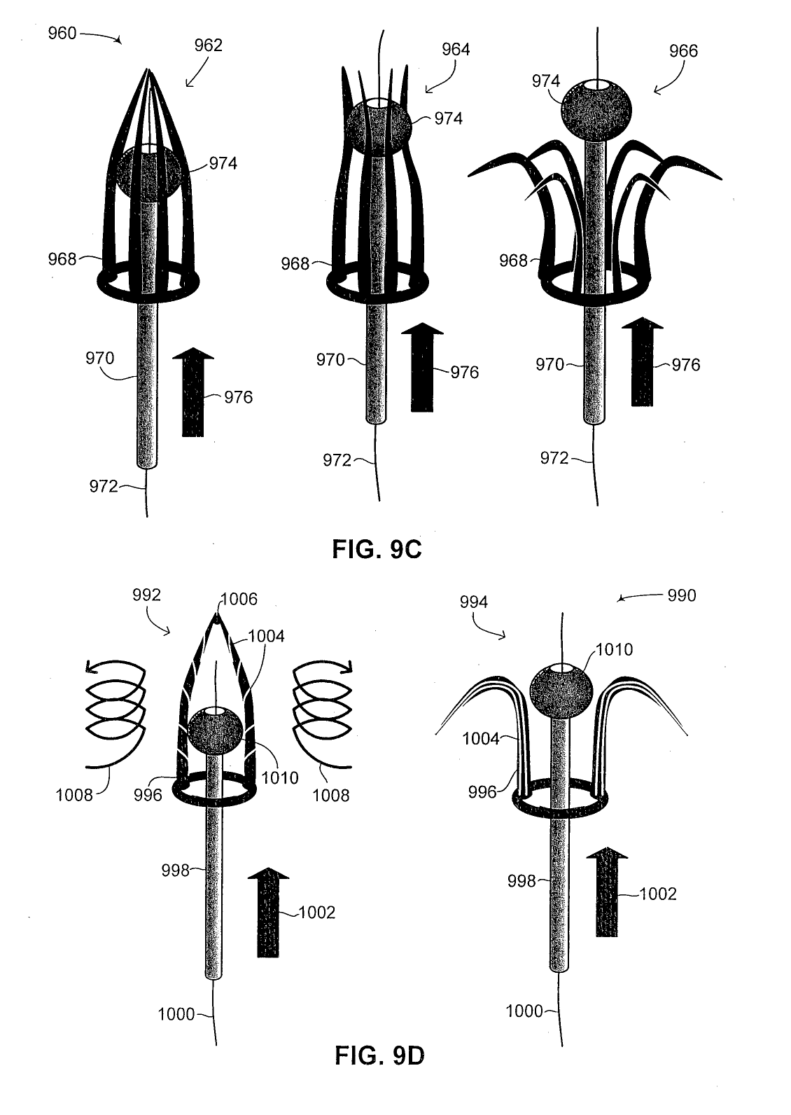

As shown in view 768, suture structure is in a closed state. Suture structure 756 can be kept in its closed state by a variety of mechanisms, as explained below in FIGS. 8A-8C and 9A-9D. In the closed state, plurality of distal thorns 758 and plurality of proximal thorns 760 may be twisted or bent out of their natural resting state to be placed in the arrangement of plurality of distal thorns 758 and plurality of proximal thorns 760 as shown in view 768. As described below in FIG. 7B, once released from the closed state, plurality of distal thorns 758 and plurality of proximal thorns 760 assume their open state, which may be their natural resting state. Plurality of distal thorns 758 and plurality of proximal thorns 760 can return to their natural resting state either due to suture structure 756 being made from a shape memory alloy, such as Nitinol, or from a metal alloy having sufficient elasticity such that a restorative force is exerted on plurality of distal thorns 758 and plurality of proximal thorns 760 when they are in their closed state. In general, suture structure 756 is kept in its closed state until blood vessel graft 754 is inserted into LAD coronary artery 752 and into the aortic arch. Once blood vessel graft 754 is properly positioned, suture structure 756 is released from its closed state into its released state to automatically suture blood vessel graft 754 to LAD coronary artery 752 and the aortic arch.

FIG. 7B shows two views of the suture structure. A view 782 shows suture structure 756 in its released state, after blood vessel graft 754 was inserted into LAD coronary artery 752. View 782 shows how suture structure 756 can be used to automatically suture blood vessel graft 756 with LAD coronary artery 752. It is noted that LAD coronary artery 752 and blood vessel graft 754 are brought merely as examples in FIGS. 7A and 7B and that suture structure 756 can be used to suture any two vessels in the body together, such as blood vessels, grafts or both. A view 784 shows a perspective representation of suture structure 756 in its released state. With reference to view 782, the protruding portion of plurality of distal thorns 758 was used to pierce the wall (not labeled) of LAD coronary artery 752, thereby forming a puncture (not shown). Using a delivery catheter (not shown), the distal end (not labeled) of blood vessel graft 754, including the protruding portion of plurality of distal thorns 758, is navigated into the puncture. At this point, suture structure is placed into its open state. Together, plurality of distal thorns 758 and plurality of proximal thorns 760 substantially open up into a ‘C’-like shape. Plurality of distal thorns 758 pierce the wall of LAD coronary artery 752 at a plurality of positions, shown as positions 786A and 786B. Plurality of proximal thorns 760 pierce the wall of blood vessel graft 754 at a plurality of positions, shown as positions 788A and 788B. Suture structure 756 thus automatically stitches and sutures blood vessel graft 754 to LAD coronary artery 752. Plurality of distal thorns 758 substantially stitches and sutures blood vessel graft 754 to LAD coronary artery 752 whereas plurality of proximal thorns 760 substantially stabilizes suture structure 756 within blood vessel graft 754. In general, once blood vessel graft 754 is coupled with LAD coronary artery 752 by sutures or by a suture structure, thus performing an anastomosis, various cells of the body (not shown) permanently couple blood vessel graft 754 with LAD coronary artery 752 over time.

With reference to view 784, in the open or released state, plurality of distal thorns 758 and plurality of proximal thorns 760 together form a ‘C’-like shape. The arrangement of plurality of distal thorns 758 and plurality of proximal thorns 760 as shown in view 784 represents the natural resting state of plurality of distal thorns 758 and plurality of proximal thorns 760. When plurality of distal thorns 758 and plurality of proximal thorns 760 are positioned in the arrangement shown in view 768 (FIG. 7A), they are under mechanical pressure due to a restorative force. Due to the path taken by each one of plurality of distal thorns 758 and plurality of proximal thorns 760 when each one of plurality of distal thorns 758 and plurality of proximal thorns 760 moves from its closed state to its open state, suture structure 756 is generally used when blood vessel graft 754 is placed perpendicular to LAD coronary artery 752.

Reference is now made to FIG. 7C, which is a perspective schematic illustration of another suture structure used to suture a blood vessel graft to a blood vessel, generally referenced 800, constructed and operative in accordance with another embodiment of the disclosed technique. Suture structure 800 is substantially similar to suture structure 756 (FIGS. 7A and 7B) and includes a connector 806, a plurality of distal thorns 802 and a plurality of proximal thorns 804. Suture structure 800 has a variable sized radius, shown by an arrow 808. Connector 806 of suture structure 800 is thus flexible. Flexibility in connector 806 is possible, for example, by fabricating connector 806 as a stent (not shown), or by making connector 806 from a shape memory alloy.

Reference is now made to FIG. 7D, which shows orthogonal projections of further suture structures used to suture a blood vessel graft to a blood vessel, generally referenced 820, constructed and operative in accordance with a further embodiment of the disclosed technique. FIG. 7D shows a single thorn 822 in its open state. A side orthogonal view 821 of single thorn 822 shows single thorn 822 to be in a ‘C’-like shape. The ‘C’-like shape shown in side orthogonal view 821 may have a plurality of configurations when viewed from a front orthogonal view, which is achieved by viewing single thorn 822 in the direction of an arrow 824. In one configuration, as shown in a front orthogonal view 826, single thorn 822 appears as a straight line. In this configuration, when single thorn 822 moves from its closed state to its open state, it substantially twists and bends in a two dimensional plane. Thus in this configuration, in side orthogonal view 821, the tips of single thorn 822 twist and bend in a plane coinciding with the plane of FIG. 7D. In another configuration, as shown in a front orthogonal view 828, single thorn 822 appears as having an ‘S’-like shape. In this configuration, when single thorn 822 move from its closed state to its open state, it substantially twists and bends in three dimensions, with a first tip 830A of single thorn 822 twisting in one direction and a second tip 830B of single thorn 822 twisting in another direction. Thus in this configuration, in side orthogonal view 821, the tips of single thorn 822 may twist and bend outside the plane coinciding with the plane of FIG. 7D. For example, one tip of single thorn 822 may twist and bend into the plane of FIG. 7D while the other tip of single thorn 822 may twist and bend out of the plane of FIG. 7D. Both of front orthogonal views 826 and 828 are views of single thorn 822 along the direction of arrow 824.

Reference is now made to FIGS. 8A-8C, which are schematic illustrations showing a first mechanism for opening the suture structure of FIGS. 7A-7D, generally referenced 850, 880 and 890 respectively, constructed and operative in accordance with another embodiment of the disclosed technique. The use of this first mechanism is shown progressively in FIGS. 8A-8C. Identical elements in FIGS. 8A-8C are labeled using identical numbering. With reference to FIG. 8A, a blood vessel graft 852 is shown loaded with a distal suture structure 858, a proximal suture structure 860, a delivery catheter 854, a guidewire 856 and a sheath 862. Distal suture structure 858 and proximal suture structure 860 are substantially similar to suture structure 756 (FIGS. 7A and 7B) and each respectively include a plurality of distal thorns 864 and 868 and a plurality of proximal thorns 866 and 870. It is noted that in another embodiment, distal suture structure 858 and proximal suture structure 860 may be manufactured to have only plurality of distal thorns 864 and 868 (not shown). In this embodiment, the distal suture structure and the proximal suture structure may be built-in to blood vessel graft 852 (not shown), for example when blood vessel graft 852 is an artificial graft. Guidewire 856 is placed within delivery catheter 854. Delivery catheter 854 is placed within distal suture structure 858 and proximal suture structure 860. Distal suture structure 858 and proximal suture structure 860 are placed within blood vessel graft 852. Sheath 862 is placed over blood vessel graft 852. It is noted that in another embodiment, sheath 862 could be placed between blood vessel graft 852 and distal suture structure 858 and proximal suture structure 860 (not shown).

In FIGS. 8A-8C, plurality of distal thorns 864 and 868 and plurality of proximal thorns 866 and 870 are fabricated from a shape memory alloy, such as Nitinol. Another example of a shape memory alloy which can be used is Flexinol®, which is produced by the company Dynalloy, Inc. Plurality of distal thorns 864 and 868 and plurality of proximal thorns 866 and 870 are manufactured to have a resting natural shape as shown in FIG. 8C. Initially, plurality of distal thorns 864 and 868 and plurality of proximal thorns 866 and 870 are forced into the shape of distal suture structure 858 and proximal suture structure 860 as shown in FIG. 8A. Distal suture structure 858 is placed in the distal end (not labeled) of blood vessel graft 852 and proximal suture structure 860 is placed in the proximal end (not labeled) of blood vessel graft 852. Sheath 862 is then placed over blood vessel graft 852. Sheath 862 may be made from a hard plastic or metal. Sheath 862 substantially locks plurality of distal thorns 864 and 868 and plurality of proximal thorns 866 and 870 in their closed state and prevents them from self-expanding into their open state.

With reference to FIG. 8B, once the distal end of blood vessel graft is properly placed within a blood vessel (not shown) sheath 862 is pulled back in a proximal direction, as shown by an arrow 882. As sheath 862 is pulled back, distal suture structure 858 is placed into its open state as plurality of distal thorns 864 and plurality of proximal thorns 866 return to their natural resting shape. Plurality of distal thorns 864 pierces the blood vessel (not shown) whereas plurality of proximal thorns 866 pierces blood vessel graft 852. Plurality of proximal thorns 866 may turn out sideways when sheath 862 is pulled back. The sideways movement of each of plurality of proximal thorns 866 may be in a given direction. The sideways movement may also be such that neighboring ones of plurality of proximal thorns 866 turn into each other. With reference to FIG. 8C, once the proximal end of blood vessel graft 852 is properly placed within another blood vessel (not shown), such as the aortic arch (not shown), sheath 862 is pulled back further in a proximal direction, as shown by arrow 882, until it has been pulled off of blood vessel graft 852. As sheath 862 is pulled back off of blood vessel graft 852, proximal suture structure 860 is placed into its open state as plurality of distal thorns 868 and plurality of proximal thorns 870 return to their natural resting shape. Plurality of distal thorns 868 pierces the other blood vessel (not shown) whereas plurality of proximal thorns 870 pierces blood vessel graft 852. As mentioned above, plurality of proximal thorns 870 may also turn out sideways when sheath 862 is pulled back. In the mechanism shown in FIGS. 8A-8C, distal suture structure 858 and proximal suture structure 860 substantially resemble a self-expanding stent in principle of operation. It is noted that in another embodiment of the disclosed technique, plurality of proximal thorns 866 and 870, or at least a portion of plurality of proximal thorns 866 and 870 may be manufactured already in their open state, thus piercing blood vessel graft 852. Such may be the case if blood vessel graft 852 is an artificial graft.

Reference is now made to FIG. 9A, which is a schematic illustration showing a second mechanism for opening the suture structure of FIGS. 7A-7D, generally referenced 910, constructed and operative in accordance with a further embodiment of the disclosed technique. FIG. 9A shows a plurality of thorns 916 is a closed state 912 and an open state 914. Plurality of thorns 916 are manufactured to have a natural resting position as shown in open state 914. Plurality of thorns 916 are placed in their closed state 912 and then surrounded by a spring 918. Spring 918 is made from a shape memory alloy, such as Nitinol or Flexinol® and is coupled with a voltage source 922 by a set of electrodes 920. Each one of set of electrodes 920 is coupled with a respective end of spring 918. In closed state 912, voltage source 922 is off, thereby providing no voltage to spring 918. To move plurality of thorns 916 into their open state, voltage source 922 is turned on, thereby providing electrical current to electrodes 920 and thus to spring 918. The electrical current provided to electrodes 920 generates heat in spring 918. The generated heat causes spring 918 to deform, as shown in open state 914. As spring 918 deforms, plurality of thorns 916 resume their natural resting position. Once plurality of thorns 916 is deployed, voltage source 922 can be turned off and spring 918 can be removed from a heart bypass surgery suturing apparatus of the disclosed technique (not shown) in which it was initially placed.

Reference is now made to FIG. 9B, which is a schematic illustration showing a third mechanism for opening the suture structure of FIGS. 7A-7D, generally referenced 940, constructed and operative in accordance with another embodiment of the disclosed technique. FIG. 9B shows a plurality of thorns 942 is a closed state 946 and an open state 948. Plurality of thorns 942 is manufactured from a shape memory alloy, such as Nitinol or Flexinol® and is coupled with a voltage source 944. In closed state 946, voltage source 944 is off, thereby providing no voltage to plurality of thorns 942. With a lack of sufficient heat, plurality of thorns 942 maintains its closed state shape, which represents the martensite phase of plurality of thorns 942. To move plurality of thorns 942 into its open state, voltage source 944 is turned on, thereby providing electrical current to plurality of thorns 942. The electrical current provided by voltage source 944 generates heat in plurality of thorns 942. The generated heat causes plurality of thorns 942 to deform into their open state, as shown in open state 948, which represents the austenite phase of plurality of thorns 942. Once in their open state, plurality of thorns 942 maintain their natural resting shape even after voltage source 944 is turned off and no more heat is provided to plurality of thorns 942.

Reference is now made to FIGS. 9C-9D, which are schematic illustrations showing a fourth mechanism for opening the suture structure of FIGS. 7A-7D, generally referenced 960 and 990 respectively, constructed and operative in accordance with a further embodiment of the disclosed technique. With reference to FIG. 9C, three progressive views, labeled 962, 964 and 966 show a fourth mechanism for opening the suture structure of FIGS. 7A-7D. View 962 shows a suture structure 968 in a closed state. Suture structure 968 is held closed by a dab of glue (not shown) as described below in FIG. 9D. Suture structure 968 may be manufactured from a shape memory alloy. A guiding catheter 970, which includes a ball 974 at its distal end (not labeled) is pushed into suture structure 968 in the direction of an arrow 976. Ball 974 may be made of metal. Guiding catheter 970 may be replaced by a hollow handle (not shown). As shown in view 962, a guidewire 972 is inserted into guiding catheter 970. As shown in view 964, as guiding catheter 970 and ball 974 are pushed in the direction of arrow 976, ball 974 applies pressure to the inner side (not labeled) of the plurality of thorns (not labeled) of suture structure 968 and substantially breaks the seal of glue (not shown) holding suture structure 968 in its closed state. Suture structure 968 begins to untwist and bend into its open state. As shown in view 966, once ball 974 has been fully pushed through suture structure 968, suture structure 968 is released and assumes its open state. The open state of suture structure 968 represents the stable position of each of the plurality of thorns (not labeled) of suture structure 968.

With reference to FIG. 9D, two views of a suture structure 996 are shown. A view 992 shows suture structure 996 held in its closed state and a view 994 shows suture structure in its open state. Suture structure 996 may be made from a shape memory alloy. As shown above in FIG. 9C, a guidewire 1000 is inserted into a guiding catheter 998. Guiding catheter 998 includes a ball 1010 at its distal end (not labeled). Ball 1010 may be made out of metal. Guiding catheter 998 may be replaced by a hollow handle (not shown). Prior to placing suture structure 996 in a blood vessel graft (not shown), each thorn (not labeled) of suture structure 996 is twisted around itself in a helical direction, as shown by a plurality of arrows 1008. This can be seen by the helical nature of a plurality of white lines 1004 on view 992. This is similar to the corkscrew shape of the seeds of the erodium flower. The tips of the plurality of thorns (not labeled) of suture structure 996 are held together by a dab, or tiny drop of glue 1006. The glue may be a biocompatible glue. The sealing force of dab of glue 1006 is slightly stronger than the restorative force within suture structure 996. The restorative force in suture structure 996 comes about because suture structure 996 is made from a shape memory alloy which has a natural tendency to return to its natural resting shape. The restorative force in suture structure 996 also comes about because each thorn in suture structure 996 is twisted around itself. As shown in view 994, guiding catheter 998 and ball 1010 are pushed through suture structure 996 in the direction of an arrow 1002. Ball 1010 applies a mechanical force (not shown) against the inner side of the plurality of thorns (not labeled) of suture structure 996. When the mechanical force is sufficient, ball 1010 substantially breaks the seal of the dab of glue (not shown or labeled in view 994) which held the tips of suture structure 996 together. Without the sealing force acting against it, the restorative force in the plurality of thorns (not labeled) of suture structure 996 causes suture structure 996 to assume its open state. In particular each one of the plurality of thorns of suture structure 996 unwinds and untwists into its natural resting shape due to the “memory” it has since suture structure 996 is made from a shape memory alloy. As shown in view 994, plurality of white lines 1004 are straight, indicating that the plurality of thorns of suture structure 996 unwind when the seal of dab of glue 1006 is broken.

Any of the mechanisms described above in FIGS. 8A-9D can be used to open the suture structures described above in FIGS. 7A-7D. In general, the plurality of thorns of the suture structures of the disclosed technique are used for suturing a blood vessel graft with a blood vessel, as shown above in FIGS. 7A and 7B. As explained above, the suture structure of the disclosed technique is positioned within a vessel to be coupled with another vessel in the body, for example, a blood vessel graft (not shown) to an LAD coronary artery (not shown). The suture structure is located in the distal end, the proximal end or in both of a vessel to be coupled. A guidewire, such as guidewire 856 (FIGS. 8A-8C), guidewire 972 (FIG. 9C) or guidewire 1000 (FIG. 9D) is inserted through the suture structure. As was explained above in FIG. 6H, the guidewire substantially serves two purposes. The first is to assist in creating a puncture within a target vessel (i.e., a second vessel). The second is to guide a first vessel as well as the suture structure and other required devices according to the disclosed technique towards a second vessel, to be coupled with the first vessel. Although not shown in FIGS. 7A and 7B, a guidewire (not shown) in used to guide blood vessel graft 754 (FIG. 7A) towards LAD coronary artery 752 (FIG. 7A), as was described in FIG. 6H. As mentioned above, blood vessel graft 754 and LAD coronary artery 752 were brought merely as examples. According to the disclosed technique, the guidewire is used to guide a first vessel towards a second vessel, wherein the first vessel is to be sutured to the second vessel. The guidewire can be used to initially puncture the wall of the second vessel. Using the example of FIGS. 7A and 7B, the guidewire may be used for initially creating a puncture in the wall of LAD coronary artery 752. Once the puncture is created, the guidewire is advanced via the puncture into LAD coronary artery 752. Blood vessel graft 754 as well as suture structure 756 (FIGS. 7A-7B) are then advanced via the puncture and using the guidewire into LAD coronary artery 752 until the tips of plurality of distal thorns 758 (FIGS. 7A-7B) are through the puncture. At this point, suture structure 756 is opened up into its released state, as per one of the mechanisms described above in FIGS. 8A-9D. As suture structure 756 opens up, plurality of distal thorns 758 stitch and couple blood vessel graft 754 to LAD coronary artery 752, while plurality of proximal thorns 760 (FIGS. 7A-7B) stabilize suture structure 756 within blood vessel graft 754.

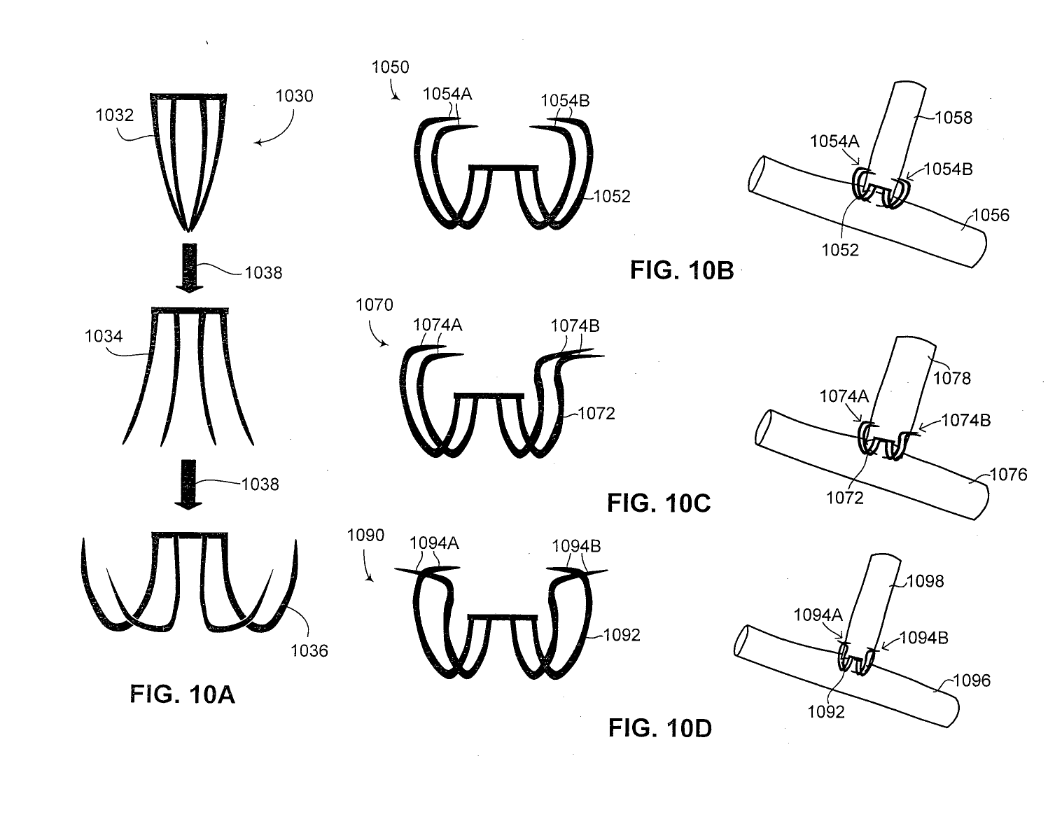

Reference is now made to FIG. 10A, which is a schematic illustration showing the transition of a suture structure from a closed state to an open state, generally referenced 1030, constructed and operative in accordance with another embodiment of the disclosed technique. The suture structure shown in FIG. 10A is substantially similar to suture structure 756 (FIGS. 7A-7B). The suture structure in FIG. 10A is shown in a first state 1032, a second state 1034 and a third state 1036. Transitions between states are shown via a plurality of arrows 1038. First state 1032 shows the suture structure in a closed state, with the tips (not labeled) of the suture structure substantially touching one another. Second state 1034 shows the suture structure in a transition state, as the tips of the suture structure being to open up and to return to their natural resting shape. Third state 1036 shows the suture structure in an open state. It is noted that if the suture structure of the disclosed technique is made from a shape memory alloy, then the suture structure can be designed having a pre-defined suturing shape. Once the suture structure is in its open state, it may continue to open up until it assumes its pre-defined suturing shape. Examples of possible pre-defined suturing shapes are shown below in FIGS. 10B-10D.

Reference is now made to FIGS. 10B-10D, which are schematic illustrations showing a plurality of pre-defined suturing shapes of the suture structure of FIG. 10A, generally referenced 1050, 1070 and 1090 respectively, constructed and operative in accordance with a further embodiment of the disclosed technique. The pre-defined suturing shapes shown in FIGS. 10B-10D represent different final twists and bends of the plurality of distal thorns and proximal thorns of the suture structure which strengthen and secure the coupling and stitching of the suture structure. After the suture structure assumes its open shape, the pre-defined suturing shapes represent the final open states of the suture structure when the suture structure is made from a shape memory alloy.

With reference to FIG. 10B, a first pre-defined suturing shape 1050 for a suture structure 1052 is shown. In first pre-defined suturing shape 1050, the tips of a plurality of thorns (not labeled) curve and bends inwards, as shown by a first portion of tips 1054A and a second portion of tips 1054B. FIG. 10B also shows suture structure 1052 in its pre-defined suturing shape when deployed in a patient (not shown) to couple a blood vessel graft 1058 with a LAD coronary artery 1056, for example. With reference to FIG. 10C, a second pre-defined suturing shape 1070 for a suture structure 1072 is shown. In second pre-defined suturing shape 1070, a portion of the tips of a plurality of thorns (not labeled) curves and bends inwards, as shown by a first portion of tips 1074A and a portion of the tips of the plurality of thorns curves and bends outwards, as shown by a second portion of tips 1074B. FIG. 100 also shows suture structure 1072 in its pre-defined suturing shape when deployed in a patient (not shown) to couple a blood vessel graft 1078 with a LAD coronary artery 1076, for example. With reference to FIG. 10D, a third pre-defined suturing shape 1090 for a suture structure 1092 is shown. In third pre-defined suturing shape 1090, a portion of the tips of a plurality of thorns (not labeled) curves and bends inwards in a hook shape whereas a portion of the tips of the plurality of thorns curves and bends outwards. In FIG. 10C, the portion of the tips which curves and bends inwards is substantially adjacent to one another and the portion of the tips which curves and bends outwards is also substantially adjacent to one another. In FIG. 10D, neighboring tips curve and bend inwards and outwards into each other, as shown by a portion of tips 1094A and a portion of tips 1094B, thus providing a tighter and more secure anastomosis. FIG. 10D also shows suture structure 1092 in its pre-defined suturing shape when deployed in a patient (not shown) to couple a blood vessel graft 1098 with a LAD coronary artery 1096, for example. It is noted that other pre-defined suturing shapes are possible and are a matter of design choice. As seen in FIGS. 10B-10D, according to the disclosed technique, the suture structure of the disclosed technique substantially securely sutures a blood vessel graft (such as blood vessel graft 1058) with another blood vessel (such as LAD coronary artery 1056) by at least puncturing the blood vessel from the inside once and the blood vessel graft from the outside once.

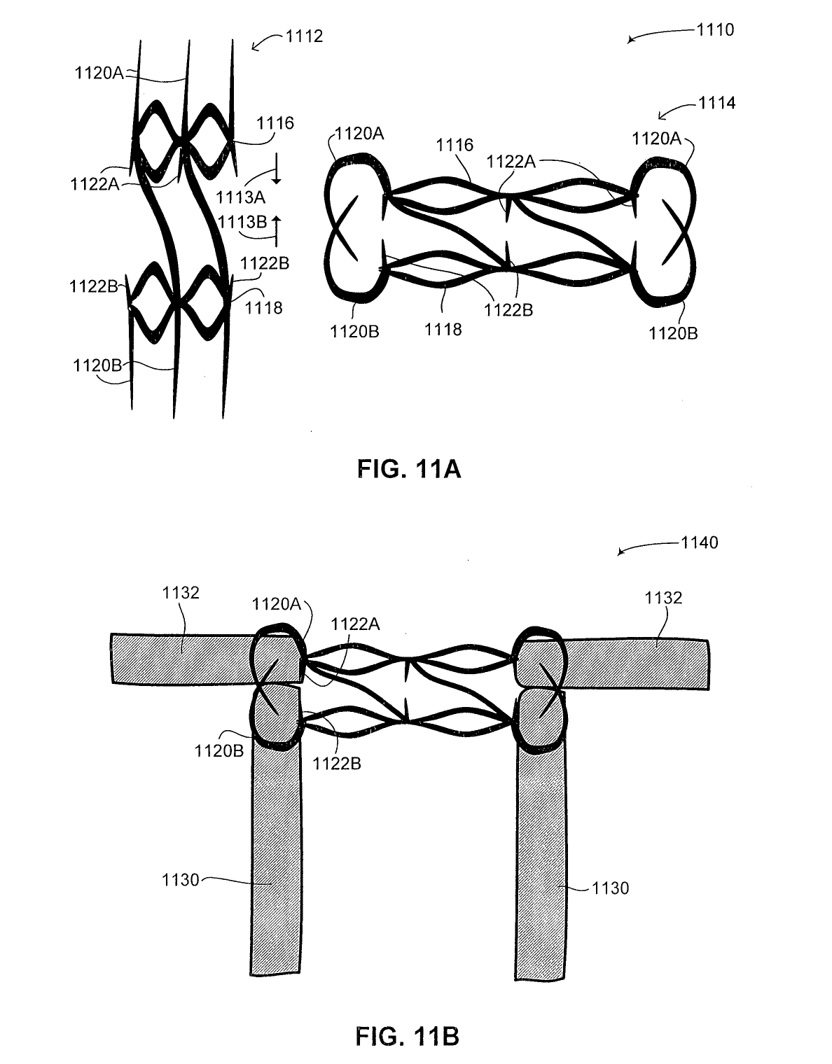

Reference is now made to FIG. 11A, which is a schematic illustration of a stitching crown suture structure, generally referenced 1110, constructed and operative in accordance with another embodiment of the disclosed technique. Stitching crown suture structure 1110 shows another embodiment of the suture structure of the disclosed technique and can be used to suture two vessels together, such as a blood vessel (not shown) and a blood vessel graft (not shown). Stitching crown suture structure 1110 substantially resembles distal suture structure 160 (FIG. 2A) and proximal suture structure 162 (FIG. 2A). In FIG. 11A, stitching crown suture structure 1110 is shown in two views. In a first view 1112, stitching crown suture structure 1110 is shown in its closed state. In a second view 1114, stitching crown suture structure 1110 is shown in its released or open state. Stitching crown suture structure 1110 may be made from a flexible metal, for example from stainless steel, nickel-free stainless steel, cobalt chrome, titanium, cobalt chromium molybdenum, a bio-absorbable material and the like. Stitching crown suture structure 1110 may also be made from a shape memory alloy, for example from Nitinol. With reference to first view 1112, stitching crown suture structure 1110 includes a distal suture portion 1116 and a proximal suture portion 1118. Distal suture portion 1116 includes a plurality of thorns 1120A and a plurality of jags 1122A. Proximal suture portion 1118 includes a plurality of thorns 11208 and a plurality of jags 11228. In general, jags can be considered small or short thorns which do not bend but remain fixed in configuration. As shown, distal suture portion 1116 and proximal suture portion 1118 are substantially mirror images of one another. In general, stitching crown suture structure 1110 is cylindrical in nature and has a crown-like shape. Stitching crown suture structure 1110 may be kept in its closed state using any of the mechanisms described above in FIGS. 8A-9D. For example, a shield (not shown) or sheath (not shown) may be placed around stitching crown suture structure 1110 thereby keeping it in its closed state. This may be the case if stitching crown suture structure 1110 is made from a self-expanding metal and equipped with a mechanism (not shown) for enabling stitching crown suture structure 1110 to self-expand. A balloon catheter (not shown) may be placed within stitching crown suture structure 1110 to aid in navigating it within a patient (not shown). The balloon catheter may optionally be used to expand stitching crown suture structure 1110 into its released state. This may be the case if stitching crown suture structure 1110 is not made from a self-expanding metal but rather from a flexible metal and is equipped with a mechanism (not shown) for enabling stitching crown suture structure 1110 to open up into its open state. This was described above in FIG. 3B. As shown in FIG. 11A, in the closed state, stitching crown suture structure 1110 has a substantially long and narrow shape.

With reference to second view 1114, when stitching crown suture structure 1110 is placed in its released state, for example by an external force, such as by using a balloon catheter (not shown) or by an internal force, such as a restorative force of a self-expanding mechanism (not shown) which is released by removing a shield (not shown), distal suture portion 1116 and proximal suture portion 1118 move towards each other. Distal suture portion 1116 moves towards proximal suture portion 1118 in the direction of an arrow 1113A and proximal suture portion 1118 moves towards distal suture portion 1116 in the direction of an arrow 1113B. The movement of distal suture portion 1116 and proximal suture portion 1118 towards each other form a crown-like shape which is wider and shorter than the long and narrow shape of stitching crown suture structure 1110 as shown in first view 1112. Plurality of thorns 1120A and 1120B assume a hook-like or ‘C’-like shape when stitching crown suture structure 1110 is in its released state, similar to the ‘C’-like shape of suture structure 756 (FIG. 7B) as shown in FIG. 7B. Plurality of jags 1122A and 1122B also assume a hook-like shape when stitching crown suture structure 1110 is in its released state, although their hook-like shape is less pronounced than the hook-like shape of plurality of thorns 1120A and 1120B. In another embodiment, plurality of jags 1122A and 1122B may not change their shape when stitching crown suture structure 1110 is in its released state. In this embodiment, plurality of jags 1122A and 1122B have a static shape. Stitching crown suture structure 1110 can be used as either a distal suture structure, a proximal suture structure or both, of the disclosed technique.

Reference is now made to FIG. 11B, which is a schematic illustration of the stitching crown suture structure of FIG. 11A used to suture two vessels, generally referenced 1140, constructed and operative in accordance with a further embodiment of the disclosed technique. Identical elements in FIGS. 11A and 11B are labeled using identical numbering. FIG. 11B shows the stitching crown suture structure of FIG. 11A (not labeled in FIG. 11B) in its released state. The stitching crown suture structure sutures a first vessel 1130 to a second vessel 1132, by suturing the walls (as shown) of first vessel 1130 to the walls (as shown) of second vessel 1132. The walls of first vessel 1130 and second vessel 1132 are not hollow. First vessel 1130 is partially inserted into second vessel 1132 through a previously created puncture in the wall of second vessel 1132, by a puncturing wire (not shown) or a guidewire (not shown). First vessel 1130 may be a blood vessel graft and second vessel 1132 may be a coronary artery, such as the LAD coronary artery. In its open state, each one of plurality of thorns 1120A twists and hooks back, thus piercing the walls of both second vessel 1132 and first vessel 1130. Each one of plurality of thorns 1120B twists and hooks back, thus piercing the wall of both first vessel 1130 and second vessel 1132. Each one of plurality of jags 1122A and 1122B pierce the walls of both first vessel 1130 and second vessel 1132 via the open state configuration of the stitching crown suture structure. Plurality of jags 1122A and 1122B provide additional support and stability to the anastomosis of first vessel 1130 with second vessel 1132 by coupling the stitching crown suture structure with first vessel 1130 and second vessel 1132 as shown in FIG. 11B. It is noted that stitching crown suture structure 1140 enables a reinforced anastomosis between first vessel 1130 and second vessel 1132, as first vessel 1130 and second vessel 1132 are coupled together via two separate sets of elements, namely, plurality of jags 1122A and 1122B and plurality of thorns 1120A and 1120B. As distal suture portion 1116 (FIG. 11A) moves towards proximal suture portion 1118 (FIG. 11A), plurality of jags 1122A pierces second vessel 1132 and plurality of jags 1122B pierces first vessel 1130, thus coupling first vessel 1130 to second vessel 1132. This coupling alone is sufficient to provide an anastomosis between first vessel 1130 and second vessel 1132. According to the disclosed technique, plurality of thorns 1120A and 1120B then additionally begin to bend and pierce first vessel 1130 and second vessel 1132 thus forming a second coupling of first vessel 1130 with second vessel 1132. This second coupling strengthens the anastomosis of first vessel 1130 with second vessel 1132 and prevents any leakage of fluid from the suture of first vessel 1130 with second vessel 1132 via stitching crown suture structure 1140.

Reference is now made to FIG. 19A, which is a schematic illustration of a suture structure with a release apparatus for deploying a stitching crown showing the deployment of the stitching crown, generally referenced 1570, constructed and operative in accordance with a further embodiment of the disclosed technique. Suture structure 1570 is shown at four different stages of its release, labeled 1572A-1572D. Equivalent elements in each stage of the release of the suture structure as shown in FIG. 19A are labeled using identical numbers. Suture structure 1570 includes a tube 1574, a balloon 1576, a plurality of holding cones 1578 and a stitching crown 1580. Stitching crown 1580 includes a plurality of tips 1582 at both of its ends. Tube 1574 is coupled with balloon 1576 and is used for inflating and deflating balloon 1576. Plurality of holding cones 1578 substantially represent a release apparatus for deploying stitching crown 1580. Plurality of holding cones 1578 are positioned around both ends of tube 1574. It is noted that stitching crown 1580 is constructed from a self-expanding material, as described above in FIGS. 3A and 3B. Plurality of holding cones 1578 are constructed from a stiff material, such as a hard plastic.

In a first stage 1572A, plurality of holding cones 1578 restrain plurality of tips 1582 of stitching crown 1580. In a second stage 1572B, as tube 1574 is used to partially inflate balloon 1576, stitching crown 1580 begins to expand while plurality of holding cones 1578 still restrain plurality of tips 1582. Balloon 1576 may be partially inflated as shown in second stage 1572B in order for a blood vessel graft (not shown) to be inserted over suture structure 1570 while firmly coupling suture structure 1570 to the blood vessel graft. In a third stage 1572C, as balloon 1576 is further inflated, stitching crown 1580 expands sufficiently such that plurality of tips 1582 is released from plurality of holding cones 1578 due to the pressure which plurality of tips 1582 exerts on plurality of holding cones 1578. Since plurality of holding cones 1578 is constructed from a stiff material and plurality of tips 1582 is constructed from a self-expanding material, there is no concern that plurality of tips 1582 will break plurality of holding cones 1578 as plurality of tips 1582 expands. In a fourth stage 1572D, stitching crown 1580 has fully expanded and is fully deployed, with plurality of tips 1582 interlocking and interweaving with one another.

Reference is now made to FIG. 19B which is a schematic illustration of the suture structure of FIG. 19A with a release apparatus for deploying a stitching crown showing the deployment of the stitching crown with a blood vessel graft and a blood vessel, generally referenced 1600, constructed and operative in accordance with another embodiment of the disclosed technique. FIG. 19B shows the release of a stitching crown (not labeled) in four stages, labeled 1602A, 1602B, 1602C and 1602D. The stages shown in FIG. 19B are substantially similar to the stages shown in FIG. 19A, except that in FIG. 19B the stages show the deployment of the stitching crown on a blood vessel graft and a blood vessel. First stage 1602A is substantially similar to first stage 1572A (FIG. 19A). Second stage 1602B is substantially similar to second stage 1572B (FIG. 19A). As shown, in this stage a blood vessel graft 1604 is placed around suture structure 1600 in the direction of an arrow 1606. In general, once blood vessel graft 1604 is positioned around suture structure 1600, a balloon (not labeled) is inflated such that the stitching crown is firmly coupled with blood vessel graft 1604. Third stage 1602C is substantially similar to third stage 1572C (FIG. 19A). As shown, in this stage blood vessel graft 1604 is advanced towards a blood vessel 1608 (partially shown), in the direction of an arrow 1610. As in third stage 1572C, in third stage 1602C, a plurality of tips (not labeled) from the ends of the stitching crown have begun to deploy and as shown have partially penetrated blood vessel 1608. Fourth stage 1602D is substantially similar to fourth stage 1572D (FIG. 19A). As shown, blood vessel graft 1604 and blood vessel 1608 are coupled together, thus performing an anastomosis via the stitching crown which has now been fully deployed.

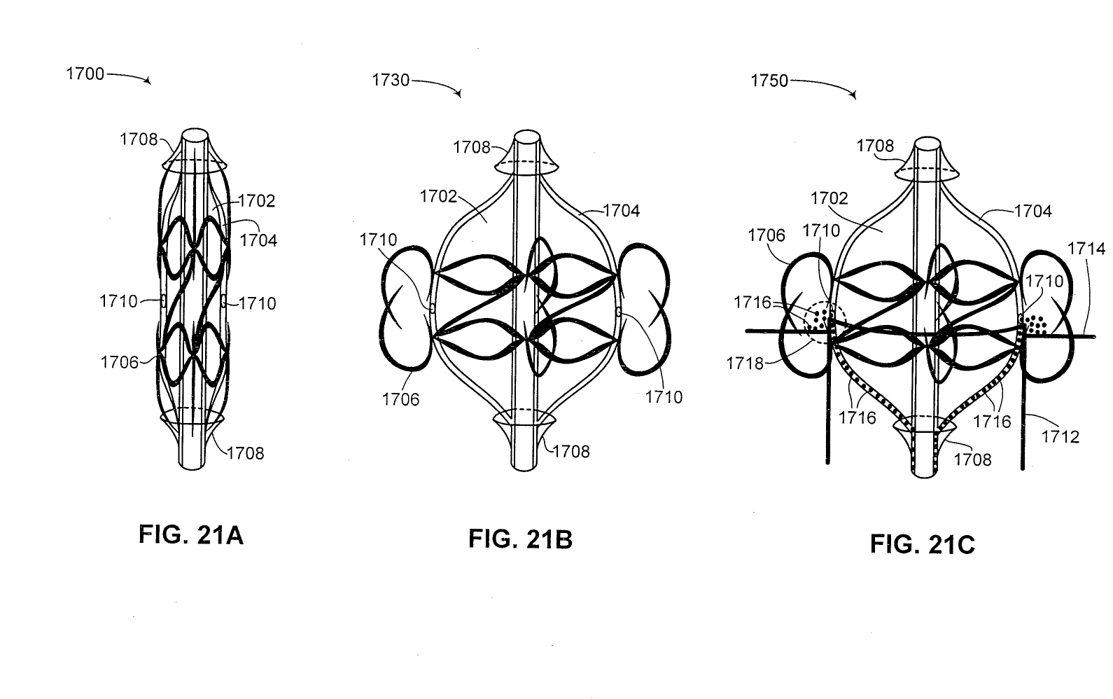

Reference is now made to FIGS. 21A-21C which are schematic illustrations of a suture structure enabling the deployment of a biological glue, generally referenced 1700, 1730 and 1750 respectively, constructed and operative in accordance with another embodiment of the disclosed technique. Equivalent elements in FIGS. 21A-21C are labeled using identical numbers. FIG. 21A show suture structure 1700. Suture structure 1700 is substantially similar to suture structure 1570 (FIG. 19A) and includes an inner balloon 1702, an outer balloon 1704, a stitching crown 1706, a plurality of holding cones 1708 and a plurality of release holes 1710 in outer balloon 1704. Unlike suture structure 1570, suture structure 1700 includes a double-walled balloon (not labeled) consisting of inner balloon 1702 and outer balloon 1704. Each one of inner balloon 1702 and outer balloon 1704 can be independently inflated and deflated. Plurality of holding cones 1708 firmly grasps the tips (not labeled) of stitching crown 1706, until stitching crown 1706 is positioned in a desired location inside a patient (not shown). Outer balloon 1704 substantially surrounds inner balloon 1702, thus forming a space (not labeled) between the outer wall (not labeled) of inner balloon 1702 and the inner wall (not labeled) of outer balloon 1704. Plurality of release holes 1710 are always open and enable a substance to be discharged from the space in outer balloon 1704. Suture structure 1700 is in a not deployed state.

In general, a blood vessel graft (not shown) is placed around suture structure 1700 and inner balloon 1702 is then partially inflated to firmly couple stitching crown 1706 (as shown above in second stage 1602B and third stage 1602C in FIG. 19B). Suture structure 1700 can then be coupled with an endoscopic delivery device (not shown) of the disclosed technique and maneuvered inside a patient (not shown) until a desired location. As shown in FIG. 21B, inner balloon 1702 is then inflated such that the tips of stitching crown 1706 are released from plurality of holding cones 1708 and stitching crown 1706 fully deploys coupling the blood vessel graft to a blood vessel (not shown). In general as inner balloon 1702 is inflated, outer balloon 1704 is inflated as well. Once stitching crown 1706 is fully deployed, a substance, such as a biological glue (not shown), can be inserted into outer balloon 1704 from an entry point (not shown) outside the patient and released, via plurality of release holes 1710, into the patient at the location where the blood vessel graft is coupled with the blood vessel, thus performing an anastomosis. The biological glue can be used to strengthen the suture formed by stitching crown 1706 for a better and more durable anastomosis.

Shown in FIG. 21C is the suture structure of FIG. 21B fully deployed on a blood vessel graft 1712, suturing blood vessel graft 1712 with a blood vessel 1714 (only partially shown). As seen, a biological glue 1716 is passed through outer balloon 1704, which flows out of outer balloon 1704 via plurality of release holes 1710. As shown in a dotted circle 1718, plurality of release holes 1710 are positioned adjacent to the area where blood vessel graft 1712 is sutured to blood vessel 1714. When biological glue 1716 is released from outer balloon 1704 is it released in the area where it can strengthen the suturing between blood vessel graft 1712 and blood vessel 1714. In general, for biological glue 1716 to flow out of outer balloon 1704, inner balloon 1702 must be slightly deflated, thereby releasing any pressure the outer surface (not labeled) of inner balloon 1702 may be placing on the inner surface (not labeled) of outer balloon 1704. Such a decrease in pressure enables biological glue 1716 to flow between inner balloon 1702 and outer balloon 1704. As mentioned above, plurality of release holes 1710 are always open. Even though a small quantity of substances inside the body of a patient (not shown) may enter into plurality of release holes 1710 as suture structure 1750 is maneuvered to its designated location inside the patient, any substance entering plurality of release holes 1710 will be discharged from outer balloon 1704 when outer balloon 1704 is filled with biological glue 1716 and biological glue 1716 is released via plurality of release holes 1710. Once suture structure 1750 is maneuvered inside the body of the patient, outer balloon 1704 filled with a sufficient quantity of biological glue 1716 such that it discharges from plurality of release holes 1710.

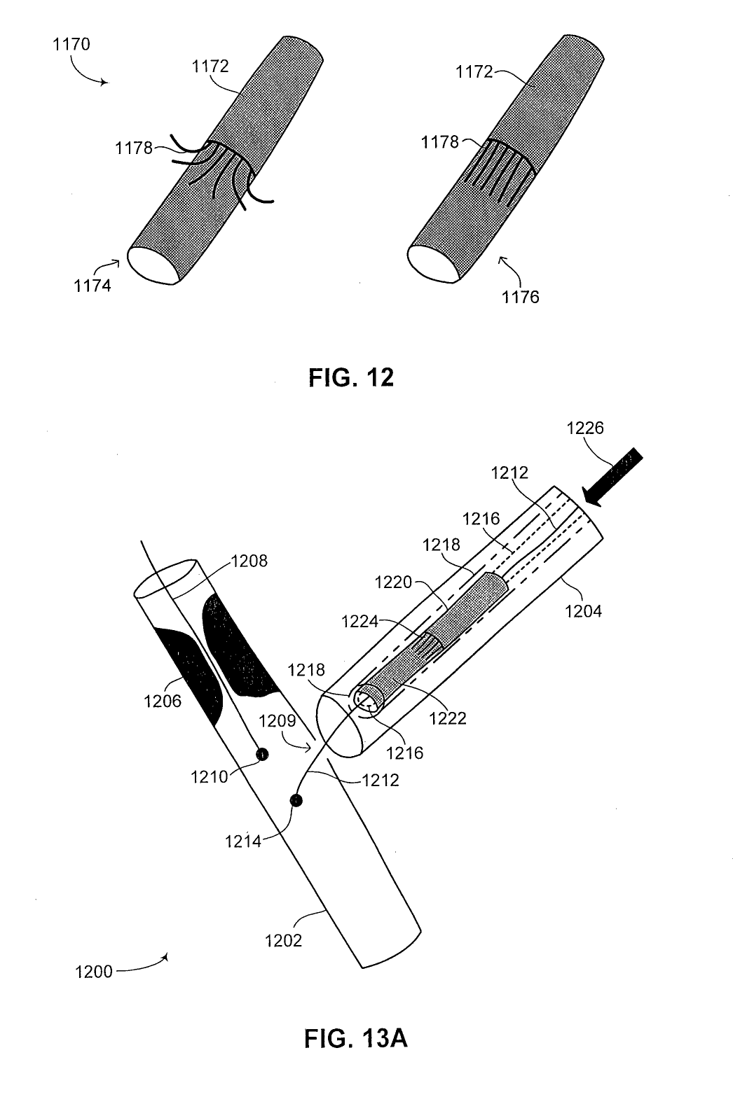

FIG. 12 is a schematic illustration of a distal suture structure, generally referenced 1170, constructed and operative in accordance with another embodiment of the disclosed technique. Distal suture structure 1170 is used for suturing a distal end of a vessel (not shown), such as a blood vessel graft (not shown), to another vessel (not shown), such as an occluded artery (not shown). Distal suture structure 1170 includes a stent 1172 and a plurality of thorns 1178. Plurality of thorns 1178 may be in the form of a crown and may resemble stitching crown suture structure 1110 (FIG. 11A). Two views of distal suture structure 1170 are shown in FIG. 12. A first view 1174 shows plurality of thorns 1178 in an open state. A second view 1176 shows plurality of thorns 1178 in a closed state. In one embodiment, stent 1172 is a self-expanding stent and plurality of thorns 1178 can also self-expand. In another embodiment, stent 1172 is deployed using a balloon (not shown) and plurality of thorns 1178 is also deployed using a balloon (not shown). In a further embodiment, a combination of a balloon (not shown) and self-expanding capabilities are used in distal suture structure 1170. For example, stent 1172 may be deployed using a balloon (not shown) where plurality of thorns 1178 may be made from a material that can self-expand. For example, plurality of thorns 1178 in this embodiment may be made from a shape memory alloy.

Reference is now made to FIGS. 13A-13D, which are schematic illustrations of the distal suture structure of FIG. 12 used in a TABG surgery of the disclosed technique, generally referenced 1200, 1230, 1240 and 1250 respectively, constructed and operative in accordance with a further embodiment of the disclosed technique. FIGS. 13A-13D schematically show another method for stitching a first vessel to a second vessel, for example a blood vessel graft to a LAD coronary artery. Identical elements in FIGS. 13A-13D are labeled using identical numbering. FIGS. 13A-13D show a continuation of the steps of the TABG surgery of the disclosed technique described above in FIGS. 6A-6H using the distal suture structure of FIG. 12. FIG. 13A shows a step substantially similar to the step of the TABG surgery of the disclosed technique shown above in FIG. 6H.

With reference to FIG. 13A, an occluded blood vessel 1202 is being sutured with a blood vessel graft 1204. Occluded blood vessel 1202 can be an LAD coronary artery. Occluded blood vessel 1202 is occluded due to a lesion 1206. In a previous step, a guidewire 1208 was navigated past lesion 1206. Guidewire 1208 includes a position sensor 1210 on its tip, which may be, for example, a radiopaque marker, an electromagnetic position sensor or other similar known position markers used in medical imaging. Blood vessel graft 1204 is inserted with a distal suture structure 1220. It is noted that blood vessel graft 1204 is navigated to a position substantially distal to lesion 1206, as shown in FIG. 13A, using an endoscopic delivery device (not shown) of the disclosed technique. It is noted that the navigation and maneuvering of blood vessel graft 1204 as described below in FIGS. 13A-13D can be performed manually by a medical practitioner or via a robotic manipulator. Distal suture structure 1220 is substantially equivalent to distal suture structure 1170 (FIG. 12) and can be placed in a closed state and an open state. Distal suture structure 1220 includes a self-expanding stent 1222 and a plurality of thorns 1224 which are also self-expanding. Self-expanding stent 1222 and plurality of thorns 1224 can both be made from a shape memory alloy such as Nitinol. Plurality of thorns 1224 can have a shape similar to stitching crown suture structure 1110 (FIG. 11A). In their closed state, plurality of thorns 1224 may be held against stent 1222 as straight lines. In their closed state, plurality of thorns 1224 may also be held against stent 1222 as twisted lines, as described above in FIG. 9D. As twisted lines, plurality of thorns 1224 may resemble the seeds of the erodium flower. Distal suture structure 1220 is surrounded by a shaft 1218 which prevents stent 1222 and plurality of thorns 1224 from self-expanding. Shaft 1218 thereby holds distal suture structure 1220 in its closed state. A catheter 1216 and a guidewire 1212 are placed within distal suture structure 1220. Guidewire 1212 includes a position sensor 1214, which may be, for example, a radiopaque marker, an electromagnetic position sensor or other similar known position markers used in medical imaging. Catheter 1216 may include a balloon (not shown). Guidewire 1212 is advanced through a puncture 1209 in occluded blood vessel 1202 to a position which is distal of lesion 1206. Puncture 1209 was created by a puncture wire (not shown) which was previously inserted through blood vessel graft 1204. In another embodiment, in some cases, guidewire 1212 can be used as the puncture wire used to create puncture 1209. Catheter 1216 is positioned within distal suture structure 1220 such that the distal ends of both catheter 1216 and distal suture structure 1220 are substantially aligned. Catheter 1216 is used to navigate distal suture structure 1220. Catheter 1216 can be used to move distal suture structure 1220 both forwards and backwards towards puncture 1209 (for example, created by guidewire 1212) and occluded blood vessel 1202. As shown in FIG. 13A by an arrow 1226, catheter 1216 is pushed in a distal direction over guidewire 1212, thereby moving distal suture structure 1220 towards puncture 1209 in occluded blood vessel 1202.

With reference to FIG. 13B, catheter 1216 is used to push a distal portion of distal suture structure 1220 through puncture 1209. Pushing distal suture structure 1220 through puncture 1209 may increase the size of puncture 1209. As shown in FIG. 13B, shaft 1218 and catheter 1216 are pushed into occluded blood vessel 1202 as well. In FIG. 13B, guidewire 1212 was advanced further into occluded blood vessel 1202. In general, distal suture structure 1220 is advanced sufficiently into occluded blood vessel 1202 until plurality of thorns 1224 substantially align with puncture 1209. Distal suture structure 1220 is advanced into occluded blood vessel 1202 while distal suture structure 1220 is still in its closed state. In general, at this point in the TABG surgery of the disclosed technique, plurality of thorns 1224 can be placed in their open or released state to suture blood vessel graft 1204 with occluded blood vessel 1202. With reference to FIG. 13C, once distal suture structure is placed sufficiently within occluded blood vessel 1202, shaft 1218 is pulled back in a proximal direction, as shown by an arrow 1232. As shaft 1218 is pulled back, stent 1222 begins to self-expand. As shown in FIG. 13C, a distal end 1234 of stent 1222 begins to self-expand to the inner diameter (not labeled) of occluded blood vessel 1202. With reference to FIG. 13D, as shaft 1218 is pulled back even further in the proximal direction shown by arrow 1232, plurality of thorns 1224 self-expand and pierce both occluded blood vessel 1202 and blood vessel graft 1204. By pulling shaft 1218 in a proximal direction even more, the distal end (not labeled) of stent 1222 self-expands in blood vessel graft 1204 (not shown). It is noted that stent 1222 may also be made from a flexible metal, such as stainless steel. In such an embodiment, stent 1222 may be expanded to the inner diameter of occluded blood vessel 1202 and the inner diameter (not labeled) of blood vessel graft 1204 by a balloon (not shown).

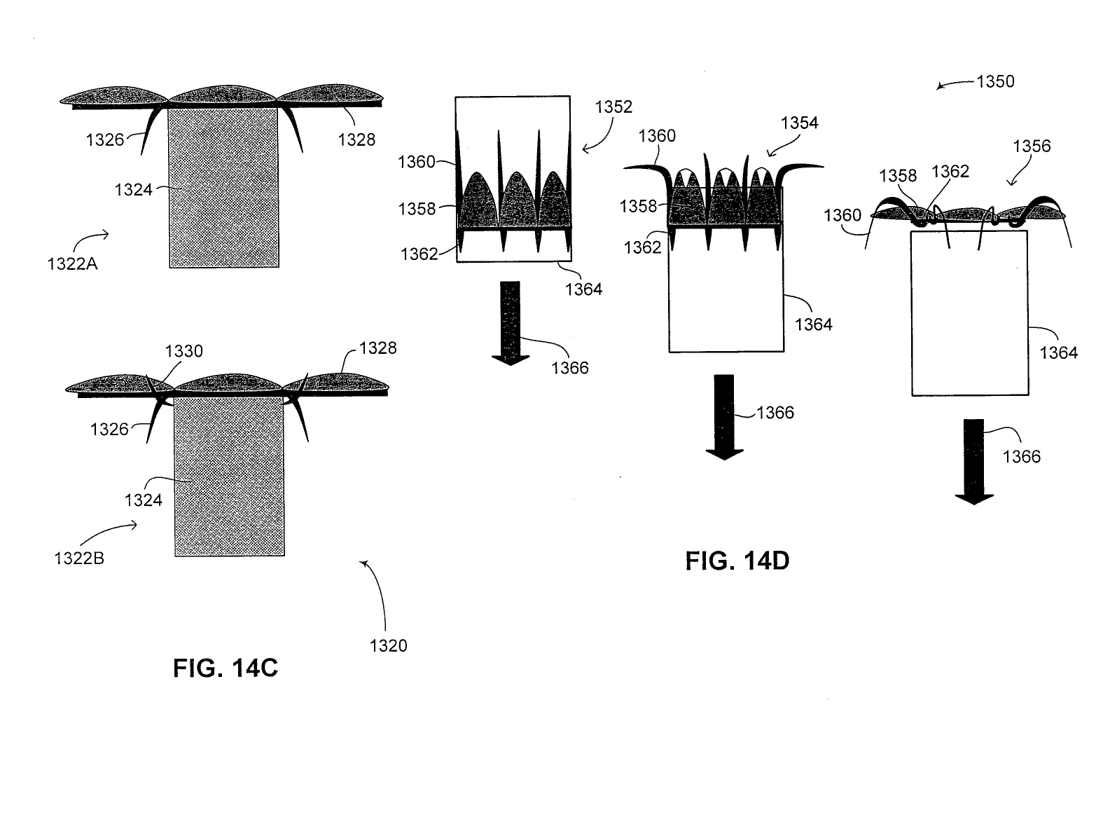

Reference is now made to FIGS. 14A-14B, which are perspective schematic illustrations of a proximal suture structure, generally referenced 1280 and 1300 respectively, constructed and operative in accordance with another embodiment of the disclosed technique. Identical elements in FIGS. 14A and 14B are labeled using identical numbering. With reference to FIG. 14A, proximal suture structure 1280 includes a stent 1282, a flexible ring 1284, a plurality of thorns 1290 and a plurality of leaves 1288. Flexible ring 1284 includes a gap 1286. Plurality of thorns 1290 and plurality of leaves 1288 are coupled with flexible ring 1284. Flexible ring 1284 is positioned at a proximal end (not labeled) of stent 1282. Proximal suture structure 1280 has two states, an open state and a closed state. As shown in FIG. 14A, proximal suture structure 1280 is in its open or released state. Proximal suture structure 1280 is used to suture a blood vessel graft (not shown) with a major blood vessel (not shown), for example a blood vessel graft with an aortic arch. Stent 1282, flexible ring 1284, plurality of thorns 1290 and plurality of leaves 1288 are all manufactured from self-expanding materials, such as shape memory alloys. In another embodiment, stent 1282, flexible ring 1284, plurality of thorns 1290 and plurality of leaves 1288 can be manufactured from a flexible material (which is not self-expanding) such as stainless steel. A balloon (not shown) can then be used to open stent 1282, plurality of thorns 1290 and plurality of leaves 1288 (not shown). Stent 1282, plurality of thorns 1290 and plurality of leaves 1288 are held in their closed position (not shown) by a shaft (not shown), similar to shaft 1218 (FIGS. 13A-13D).

With reference to FIG. 14B, the proximal suture structure of FIG. 14A is deployed within a blood vessel graft 1292 and a blood vessel 1294. Blood vessel 1294 may be, for example, an aortic arch. As shown, stent 1282 substantially extends along a proximal end of blood vessel graft 1292 and secures the proximal suture structure within blood vessel graft 1292. Plurality of leaves 1288, when in their open state, substantially support stent 1282 in blood vessel 1294. Plurality of thorns 190, when in their open state, substantially suture and secure blood vessel graft 1292 to blood vessel 1294.

Reference is now made to FIG. 14C, which is a side orthogonal schematic illustration of different embodiments of the proximal suture structure of FIG. 14A, generally referenced 1320, constructed and operative in accordance with a further embodiment of the disclosed technique. In a first embodiment 1322A, the proximal suture structure includes a stent 1324, a plurality of thorns 1326 and a plurality of leaves 1328. In a second embodiment 1322B, the proximal suture structure includes a stent 1324, a plurality of thorns 1326, a plurality of leaves 1328 and a plurality of jags 1330, which point in an opposite direction, with respect to plurality of thorns 1326, in their released state. Plurality of jags 1330 may provide extra support for the proximal suture structure when used in a patient (not shown).

Reference is now made to FIG. 14D, which is another schematic illustration of the proximal suture structure of FIG. 14A, generally referenced 1350, constructed and operative in accordance with another embodiment of the disclosed technique. FIG. 14D shows a possible mechanism for keeping the proximal suture structure of FIG. 14A in its closed state and for opening it up to its released state. In a first step 1352, the proximal suture structure, including a plurality of leaves 1358, a plurality of thorns 1360 and a plurality of jags 1362, is kept in its closed state by a shaft 1364. In order to put the proximal suture structure in its open state, shaft 1364 is pulled in the direction of an arrow 1366. In a second step 1354, shaft 1364 has been pulled back to partially uncover plurality of leaves 1358 and plurality of thorns 1360. As shaft 1364 is pulled back in second step 1354, plurality of thorns 1360 and plurality of leaves 1358 begin twisting and bending into their open state. In a third step 1356, when shaft 1364 has been fully pulled back from the proximal end (not labeled) of the proximal suture structure, plurality of leaves 1358, plurality of thorns 1360 and plurality of jags 1362 all assume their open state.

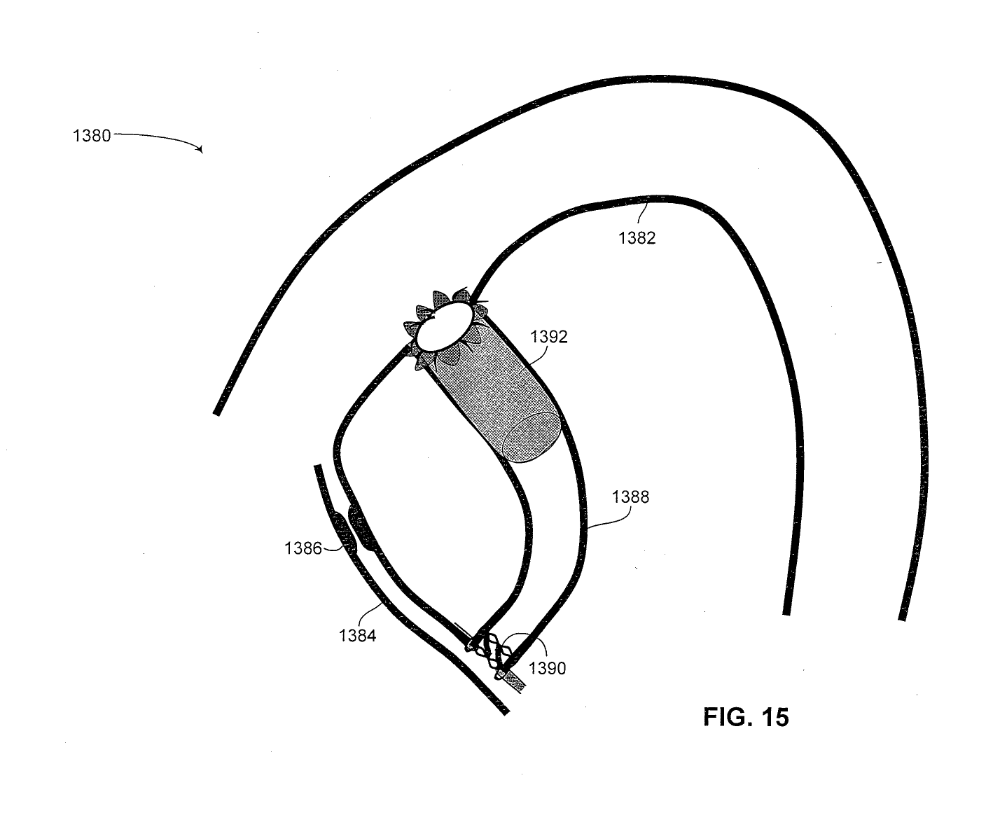

Reference is now made to FIG. 15, which is a schematic illustration of a heart bypass surgery suturing apparatus fully deployed in a patient, generally referenced 1380, constructed and operative in accordance with a further embodiment of the disclosed technique. FIG. 15 shows an aortic arch 1382 and an occluded blood vessel 1384 coupled together by a bypass route using a blood vessel graft 1388. Occluded blood vessel 1384 is occluded by a lesion 1386. Occluded blood vessel 1384 may be a coronary artery (not shown), such as the LAD coronary artery. Once deployed, blood vessel graft 1388 enables blood to flow from aortic arch 1382 into occluded blood vessel 1384 at a position on occluded blood vessel 1384 which is distal to lesion 1386. Blood vessel graft 1388 is sutured to aortic arch 1382 via a proximal suture structure 1392. Proximal suture structure 1392 is substantially similar to the proximal suture structures shown above in FIGS. 14A-14D. It is noted that proximal suture structure 1392 in FIG. 15 can be replaced by any of the suture structures of the disclosed technique described above. Blood vessel graft 1388 is sutured to occluded blood vessel 1384 via a distal suture structure 1390. Distal suture structure 1390 is substantially similar to the stitching crown suture structures shown above in FIGS. 11A-11B. It is noted that distal suture structure 1390 in FIG. 15 can be replaced by any of the suture structures of the disclosed technique described above. As mentioned above, once the blood vessel graft is fully deployed and sutured according to the disclosed technique, a contrast dye is injected into the blood vessel graft to verify the flow of liquids from the aortic arch to the occluded blood vessel via the bypass.

It is noted that the overall TABG surgery of the disclosed technique described above, in particular in FIGS. 6A-6H and 13A-13D can be executed multiple times for implanting a plurality of blood vessel grafts in a single patient. After the TABG surgery of the disclosed technique is executed once, it may be executed additional times by performing the entire procedure again from the first step shown in FIG. 6A. In another embodiment, after the TABG surgery of the disclosed technique is executed once, it may be executed additional times using the same blocking balloon (not shown). In this embodiment, the blocking balloon is slightly deflated such that is can be rotated, moved or both in order to be repositioned in the aorta of a patient. The blocking balloon is then fully inflated and the TABG surgery of the disclosed technique can be executed an additional time by inserting another blood vessel graft through the first tube (not shown) of the blocking balloon using the endoscopic delivery device (not shown) of the disclosed technique. For example, after blood vessel graft 1388 is sutured between aortic arch 1382 and occluded blood vessel 1384, another blood vessel graft (not shown) may be sutured between aortic arch 1382 and another blood vessel (not shown), such as a right coronary artery (herein abbreviated RCA) of the patient. It is further noted that the disclosed technique can be used in a hybrid manner with other known techniques for treating coronary heart disease. For example, after the TABG surgery of the disclosed technique is executed for coupling a blood vessel graft to a patient's heart to bypass an occlusion, the blocking balloon of the disclosed technique may be removed. A known percutaneous coronary intervention procedure may then be performed via the original incision made within the patient to insert a stent in another blood vessel of the patient's heart. For example, the TABG surgery of the disclosed technique can be used to bypass an occlusion in the right coronary artery of the patient, and then a stent may be inserted in the left anterior descending coronary artery using a known percutaneous coronary intervention procedure via the same incision in the patient.

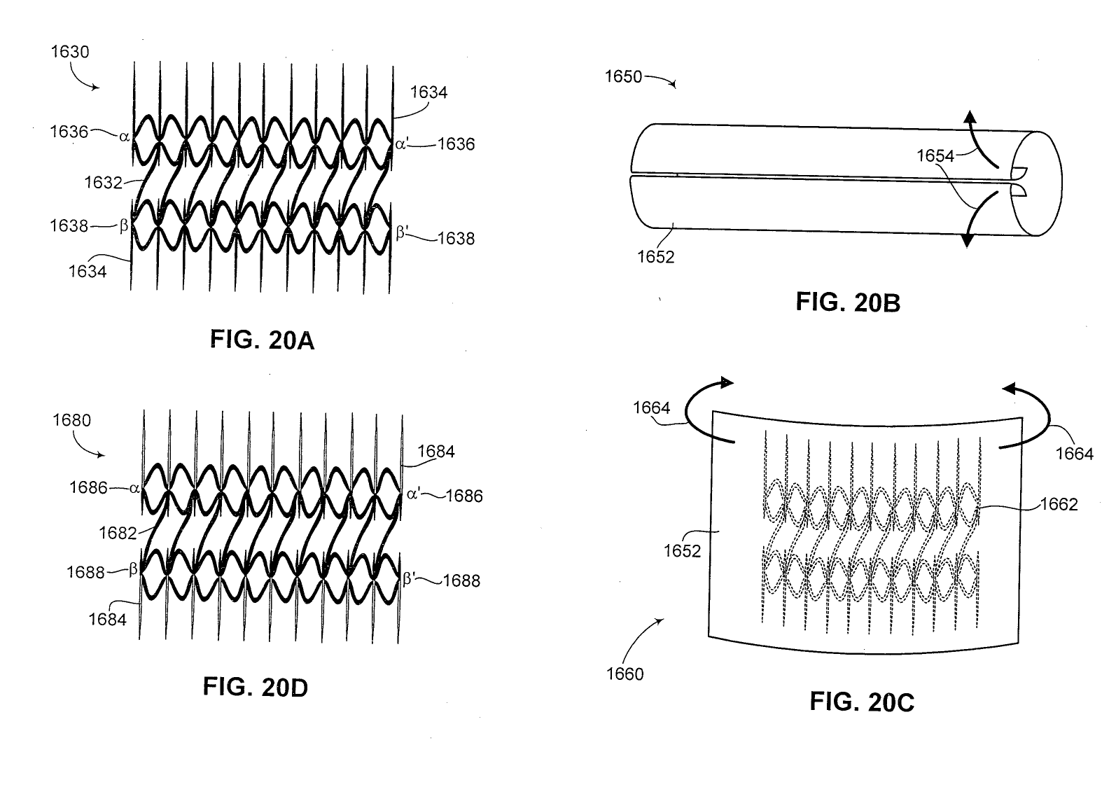

Reference is now made to FIG. 20A which is a first schematic illustration of a manufacturing method of a suturing structure, generally referenced 1630, constructed and operative in accordance with a further embodiment of the disclosed technique. In general suturing structure 1630 is constructed from a shape memory alloy or from a metal. In the illustration shown in FIG. 20A, suturing structure 1630 includes a central portion 1632 and a plurality of jags 1634. The shape of suturing structure 1630 as shown in FIG. 20A may be cut out from a flat sheet of metal (not shown) as a single shape. The open sides 1636 and 1638 of suturing structure 1630 are then coupled with one another to form the circular (not shown) shape of the suturing structure of the disclosed technique, for example as shown above in FIGS. 7A and 7B. The circular shape is formed by coupling α to α′ and β to β′ via glue, adhesives, weld spots and the like.

Reference is now made to FIGS. 20B-20C which are second and third schematic illustrations of the manufacturing method of the suturing structure of FIG. 20A, generally referenced 1650 and 1660 respectively, constructed and operative in accordance with another embodiment of the disclosed technique. Equivalent elements in FIGS. 20B and 20C are labeled using identical numbers. FIG. 20B shows a first step 1650 of the manufacturing process. In this step, a shape memory alloy 1652 is placed in its memory shape. Shape memory alloy 1652 may be, for example, Nitinol or Flexinol®. As shown in FIG. 20B, the memory shape is circular. Once shape memory alloy 1652 is in its memory shape, shape memory alloy 1652 is unfurled into a flat sheet as shown by a plurality of arrows 1654. FIG. 20C shows a second step 1660 of the manufacturing process. As shown, shape memory alloy 1652 is now unfurled as a flat sheet. As a flat sheet, a stitching crown shape 1662 is cut out of shape memory alloy 1652. Stitching crown shape 1662 is then returned to its memory shape, as shown by a plurality of arrows 1664. As the memory shape was circular, stitching crown shape 1662 in its memory shape (not shown) will be circular.

Reference is now made to FIG. 20D which is a fourth schematic illustration of the manufacturing method of the suturing structure of FIG. 20A, generally referenced 1680, constructed and operative in accordance with a further embodiment of the disclosed technique. Suturing structure 1680 includes a central portion 1682 and a plurality of jags 1684, similar to suturing structure 1630 (FIG. 20A). Unlike suturing structure 1630 which is constructed from a single material, in suturing structure 1680, central portion 1682 and plurality of jags 1684 are constructed from different materials. For example, central portion 1682 may be constructed from a memory shape alloy and plurality of jags 1684 may be constructed from a metal such as stainless steel, nickel cobalt and the like. Plurality of jags 1684 are coupled with central portion 1682 via gluing, an adhesive, welding and the like. The shapes of central portion 1682 and plurality of jags 1684 may be cut out from a flat sheet of metal (not shown) as shown above in FIG. 20A or from an unfurled sheet of a shape memory alloy, as shown above in FIGS. 20B-20C. The open sides 1686 and 1688 of suturing structure 1680 can then be coupled with one another, for example to form the circular (not shown) shape of the suturing structure of the disclosed technique if central portion 1682 was not constructed from a shape memory alloy. The open sides can be coupled by coupling α to α′ and β to β′ via glue, adhesives, weld spots and the like.

It will be appreciated by persons skilled in the art that the disclosed technique is not limited to what has been particularly shown and described hereinabove. Rather the scope of the disclosed technique is defined only by the claims, which follow.

1-237. (canceled)

238. Apparatus for enabling access to a lumen in a body via a vessel in said body without impeding a flow of a matter in said vessel, comprising:
an inflatable hollow cylindrical portion, for generating a matter-free area in said vessel;
a first tube, for enabling access to said matter-free area; and
a second tube, coupled with said inflatable hollow cylindrical portion, for inflating and deflating said inflatable hollow cylindrical portion,
said inflatable hollow cylindrical portion comprising:
a first opening; and
a second opening,
wherein said first tube is coupled with said first opening,
wherein said first opening is positioned in said inflatable hollow cylindrical portion in a direction substantially perpendicular to said flow of said matter,
wherein said second opening is positioned in said inflatable hollow cylindrical portion in a direction substantially parallel to said flow of said matter, thereby enabling said flow of said matter, and
wherein a distal end of said first tube enables an inner wall of said vessel to be accessed and wherein a proximal end of said first tube is external to said body.

239. The apparatus according to claim 238, wherein said inflatable hollow cylindrical portion is inflated by injecting saline solution into said second tube.

240. The apparatus according to claim 238, wherein a diameter of said first tube is larger than a diameter of said second tube.

241. The apparatus according to claim 238, wherein said first tube and said second tube are constructed from a durable material.

242. The apparatus according to claim 238, wherein said durable material is plastic.

243. The apparatus according to claim 238, wherein said inflatable hollow cylindrical portion is constructed from a flexible material.

244. The apparatus according to claim 243, wherein said flexible material is selected from the list consisting of:
silicone;
polyurethane
polyethylene;
polyvinylchloride;
polyethylene terephthalate; and
nylon.

245. The apparatus according to claim 238, wherein said first tube and said second tube are coupled together to form a catheter-like device.

246. The apparatus according to claim 238, wherein a maximum external radius of said inflatable hollow cylindrical portion, when said inflatable hollow cylindrical portion is inflated, is substantially similar to an inner radius of said vessel.

247. The apparatus according to claim 238, wherein said vessel is selected from the list consisting of:
a major blood vessel;
a peripheral blood vessel;
a trachea;
an esophagus; and
an anus.

248. The apparatus according to claim 238, wherein said matter is selected from the list consisting of:
blood;
air;
fecal matter; and
food.

249. The apparatus according to claim 238, further comprising a plurality of radiopaque markers.

250. The apparatus according to claim 249, wherein said plurality of radiopaque markers are positioned around said first opening and said second opening for aiding in navigating said apparatus in said body.

251. The apparatus according to claim 238, wherein said inflatable hollow cylindrical portion is inflated to a pressure sufficient to prevent said matter flowing through said second opening from entering said first opening.

252. The apparatus according to claim 238, further comprising a flexible ring, coupled with said first opening.

253. The apparatus according to claim 252, wherein said flexible ring is constructed from a material selected from the list consisting of:
silicon;
rubber;
plastic; and
a soft and adaptable polymer.

254. The apparatus according to claim 238, wherein at least one material is passed through said first tube to said matter-free area.

255. The apparatus according to claim 254, wherein said at least one material is selected from the list consisting of:
a tool;
a device;
a gas;
air; and
a liquid.

256. The apparatus according to claim 238, wherein a surgical tool is passed through said first tube to said matter-free area for puncturing said inner wall of said vessel while preventing said matter in said vessel flowing through said second opening from entering said first opening.

257. The apparatus according to claim 238, wherein a surgical tool is passed through said first tube to said matter-free area for performing a procedure on said inner wall of said vessel while preventing said matter in said vessel flowing through said second opening from entering said first opening.

258. Apparatus for enabling access to a lumen in a body via a vessel in said body without impeding a flow of a matter in said vessel, comprising:
an inflatable ring portion, for generating a matter-free area in said vessel;
an inflatable arch portion, coupled with said inflatable ring portion, for forming a first opening;
a first tube, for enabling access to said matter-free area; and
a second tube, coupled with said inflatable ring portion, for inflating and deflating said inflatable ring portion and said inflatable arch portion,
said inflatable ring portion comprising a second opening,
wherein said first tube is coupled with said second opening,
wherein said second opening is positioned in said inflatable ring portion in a direction substantially perpendicular to said flow of said matter,
wherein said first opening is formed in a direction substantially parallel to said flow of said matter, thereby enabling said flow of said matter, and
wherein a distal end of said first tube enables an inner wall of said vessel to be accessed and wherein a proximal end of said first tube is external to said body.

259. Apparatus for enabling access to a lumen in a body via a vessel in said body without impeding a flow of blood in said vessel, comprising:
an inflatable hollow cylindrical portion, for generating a blood-free area in said vessel;
a first tube, for enabling access to said blood-free area; and
a second tube, coupled with said inflatable hollow cylindrical portion, for inflating and deflating said inflatable hollow cylindrical portion,
said inflatable hollow cylindrical portion comprising:
a first opening; and
a second opening,
wherein said first tube is coupled with said first opening,
wherein said first opening is positioned in said inflatable hollow cylindrical portion in a direction substantially perpendicular to said flow of blood,
wherein said second opening is positioned in said inflatable hollow cylindrical portion in a direction substantially parallel to said flow of blood, thereby enabling said flow of blood, and
wherein a distal end of said first tube enables an inner wall of said vessel to be accessed and wherein a proximal end of said first tube is external to said body.

260. Method for enabling access to a lumen in a body via a vessel in said body without impeding a flow of a matter in said vessel using a blocking balloon, comprising the procedures of:
imaging said vessel for determining a point in said vessel adjacent to said lumen;
performing an incision in said body to a major vessel;
inserting an introducer in said incision for generating an entry-point into said major vessel;
percutaneously inserting said blocking balloon via said entry-point into said major vessel;
maneuvering said blocking balloon to said point in said vessel;
inflating said blocking balloon at said point, thereby generating a matter-free working space around said point, said matter-free working space being coupled with a first tube of said blocking balloon;
advancing a puncturing wire to said matter-free working space;
puncturing said vessel at said point using said puncturing wire; and
removing said puncturing wire from said matter-free working space, thereby enabling access to said lumen via said vessel.

261. The method according to claim 260, wherein said matter-free working space around said point is selected from the list consisting of:
a blood-free working space;
a fecal-free working space;
a food-free working space; and
an air-free working space.

262. The method according to claim 260, further comprising the procedure of inserting a guidewire via said entry-point into said major vessel, for assisting said maneuvering of said blocking balloon to said point.

263. The method according to claim 260, wherein said procedure of maneuvering said blocking balloon comprises the sub-procedure of imaging said blocking balloon using at least one imaging technique during said procedure of maneuvering said blocking balloon.

264. The method according to claim 260, wherein said procedure of maneuvering said blocking balloon is executed by a robotic manipulator.

265. The method according to claim 260, further comprising the procedures of:
deflating said blocking balloon;
removing said blocking balloon from said body;
removing said introducer from said body; and
sewing up said incision.

266. The method according to claim 260, further comprising the procedures of:
slightly deflating said blocking balloon;
navigating said blocking balloon to another point in said vessel;
inflating said blocking balloon at said another point, thereby generating another matter-free working space around said another point; and
repeating from said procedures of claim 23 at least once at said another point starting from said procedure of advancing said puncturing wire.

267. The method according to claim 260, further comprising the procedure of:
deflating said blocking balloon;
removing said blocking balloon from said body; and
executing a percutaneous surgical procedure in said body through said introducer.
